(19) United States
(12) Patent Application Publication
(10) Pub. No.: US 2017/0275275 A1
(43) Pub. Date: Sep. 28, 2017

(54) BENZENESULFONAMIDES USEFUL AS SODIUM CHANNEL INHIBITORS

(71) Applicant: PFIZER INC., NEW YORK, NY (US)

(72) Inventors: ALAN DANIEL BROWN, GREAT ABINGTON, CAMBRIDGE (GB); LYN HOWARD JONES, WINCHESTER, MA (US); BRIAN EDWARD MARRON, DURHAM, NC (US); DAVID JAMES RAWSON, SANDWICH, KENT (GB); THOMAS RYCKMANS, SANDWICH, KENT (GB); ROBERT IAN STORER, GREAT ABINGTON, CAMBRIDGE (GB); NIGEL ALAN SWAIN, GREAT ABINGTON, CAMBRIDGE (GB); CHRISTOPHER WILLIAM WEST, CARY, NC (US)

(21) Appl. No.: 15/310,113

(22) PCT Filed: May 29, 2015
(86) PCT No.: PCT/IB2015/054072

§ 371 Date: Nov. 10, 2016

Related U.S. Application Data
(60) Provisional application No. 62/139,266, filed on Mar. 27, 2015.
(60) Provisional application No. 62/004,935, filed on May 30, 2014.

(51) Int. Cl. C07D 417/14; C07D 285/08; A61K 31/4439; C07D 417/12; A61K 31/454; A61K 31/4545; A61K 31/496

(52) U.S. Cl. CPC C07D 417/14; A61K 31/4545; C07D 285/08; A61K 31/496; C07D 417/12; A61K 31/454; A61K 31/4439

(57) ABSTRACT

The invention relates to sulfonamide derivatives, to their use in medicine, to compositions containing them, to processes for their preparation and to intermediates used in such processes. More particularly the invention relates to a new sulfonamide Nav1.7 inhibitors of formula (I), or a pharmaceutically acceptable salt thereof, wherein X, R¹, R², R^{3a}, R^{3b} and R⁴ are as defined in the description. Nav1.7 inhibitors are potentially useful in the treatment of a wide range of disorders, particularly pain.

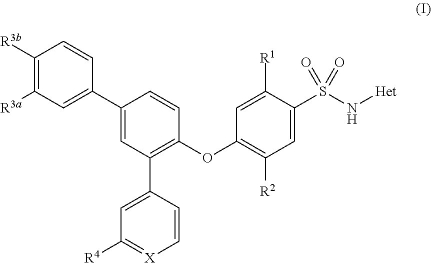

The invention relates to sulfonamide derivatives, to their use in medicine, to compositions containing them, to processes for their preparation and to intermediates used in such processes.

Voltage-gated sodium channels are found in all excitable cells including myocytes of muscle and neurons of the central and peripheral nervous system. In neuronal cells, sodium channels are primarily responsible for generating the rapid upstroke of the action potential. In this manner sodium channels are essential to the initiation and propagation of electrical signals in the nervous system. Proper and appropriate function of sodium channels is therefore necessary for normal function of the neuron. Consequently, aberrant sodium channel function is thought to underlie a variety of medical disorders (see Hubner C A, Jentsch T J, Hum. Mol. Genet., 11(20): 2435-45 (2002) for a general review of inherited ion channel disorders) including epilepsy (Yogeeswari et al., Curr. Drug Targets, 5(7): 589-602 (2004)), arrhythmia (Noble D., Proc. Natl. Acad. Sci. USA, 99(9): 5755-6 (2002)) myotonia (Cannon, S C, Kidney Int. 57(3): 772-9 (2000)), and pain (Wood, J N et al., J. Neurobiol., 61(1): 55-71 (2004)).

There are currently at least nine known members of the family of voltage-gated sodium channel (VGSC) alpha subunits. Names for this family include SCNx, SCNAx, and Naᵥx.x. The VGSC family has been phylogenetically divided into two subfamilies Naᵥ1.x (all but SCN6A) and Naᵥ2.x (SCN6A). The Nav1.x subfamily can be functionally subdivided into two groups, those which are sensitive to blocking by tetrodotoxin (TTX-sensitive or TTX-s) and those which are resistant to blocking by tetrodotoxin (TTX-resistant or TTX-r).

The Naᵥ1.7 (PN1, SCN9A) VGSC is sensitive to blocking by tetrodotoxin and is preferentially expressed in peripheral sympathetic and sensory neurons. The SCN9A gene has been cloned from a number of species, including human, rat, and rabbit and shows ˜90% amino acid identity between the human and rat genes (Toledo-Aral et al., Proc. Natl. Acad. Sci. USA, 94(4): 1527-1532 (1997)).

An increasing body of evidence suggests that Naᵥ1.7 may play a key role in various pain states, including acute, inflammatory and/or neuropathic pain. Deletion of the SCN9A gene in nociceptive neurons of mice led to a reduction in mechanical and thermal pain thresholds and reduction or abolition of inflammatory pain responses (Nassar et al., Proc Natl Acad Sci USA, 101(34): 12706-11 (2004)). In humans, Naᵥ1.7 protein has been shown to accumulate in neuromas, particularly painful neuromas (Kretschmer et al., Acta. Neurochir. (Wien), 144(8): 803-10 (2002)). Gain of function mutations of Naᵥ1.7, both familial and sporadic, have been linked to primary erythermalgia, a disease characterized by burning pain and inflammation of the extremities (Yang et al., J. Med. Genet., 41(3): 171-4 (2004), and paroxysmal extreme pain disorder (Waxman, S G Neurology. 7; 69(6): 505-7 (2007)). Congruent with this observation is the report that the non-selective sodium channel blockers lidocaine and mexiletine can provide symptomatic relief in cases of familial erythermalgia (Legroux-Crepel et al., Ann. Dermatol Venereol., 130: 429-433) and carbamazepine is effective in reducing the number and severity of attacks in PEPD (Fertleman et al, Neuron.; 52(5):767-74 (2006). Further evidence of the role of Nav1.7 in pain is found in the phenotype of loss of function mutations of the SCN9A gene. Cox and colleagues (Nature, 444(7121):894-8 (2006)) were the first to report an association between loss-of-function mutations of SNC9A and congenital indifference to pain (CIP), a rare autosomal recessive disorder characterized by a complete indifference or insensitivity to painful stimuli. Subsequent studies have revealed a number of different mutations that result in a loss of function of the SCN9A gene and and the CIP phenotype (Goldberg et al, Clin Genet.; 71(4): 311-9 (2007), Ahmad et al, Hum Mol Genet. 1; 16(17): 2114-21 (2007)).

Nav 1.7 inhibitors are therefore potentially useful in the treatment of a wide range of disorders, particularly pain, including: acute pain; chronic pain; neuropathic pain; inflammatory pain; visceral pain; and nociceptive pain.

Certain inhibitors of voltage gated sodium channels useful in the treatment of pain are known. WO 2008/118758, WO 2009/012242, WO 2010/079443, WO 2012/004706, WO2012/004714 and WO2012/004743 disclose sulphonamides.

There is, however, an ongoing need to provide new Naᵥ1.7 inhibitors that are good drug candidates.

Preferably compounds are selective Nav1.7 channel inhibitors. That is, preferred compounds show an affinity for the Nav1.7 channel over other Nav channels. In particular, they show an affinity for the Nav1.7 channel which is greater than their affinity for the Nav1.5 channel. Advantageously, compounds should show little or no affinity for the Nav1.5 channel.

Selectivity for the Nav1.7 channel over Nav1.5 may potentially lead to one or more improvements in side-effect profile, such as with regard to any cardiovascular side effects which may be associated with affinity for the Nav1.5 channel. Preferably compounds demonstrate a selectivity of 10-fold, more preferably 30-fold, most preferably 50-fold, for the Nav 1.7 channel when compared to their selectivity for the Nav1.5 channel whilst maintaining good potency for the Nav1.7 channel.

Furthermore, preferred compounds should have good aqueous solubility. They should preferably exist in a physical form that is stable, non-hygroscopic and easily formulated (e.g for parenteral administration). Ideal drug candidates should be non-toxic and demonstrate few side-effects.

We have now found new sulphonamide Nav1.7 inhibitors.

According to a first aspect of the invention there is provided a compound of formula (I)

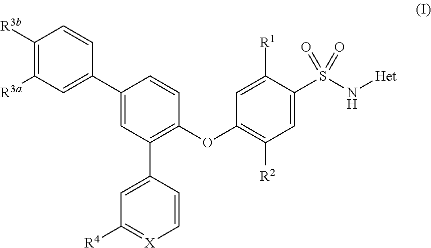

or a pharmaceutically acceptable salt thereof, wherein:
Het is ‘C-linked’ thiazolyl or thiadiazolyl;
X is CH or N;
R¹ is H or F;
R² is Cl or CN;
R^{3a} is H or CF₃;
R^{3b} is H or, when R^{3a} is H, may also be CF₃;
R⁴ is

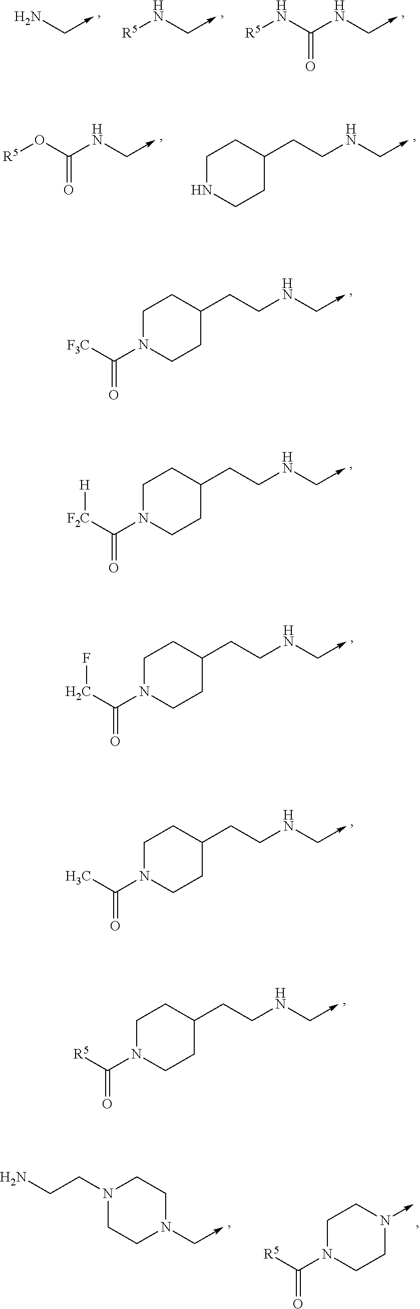

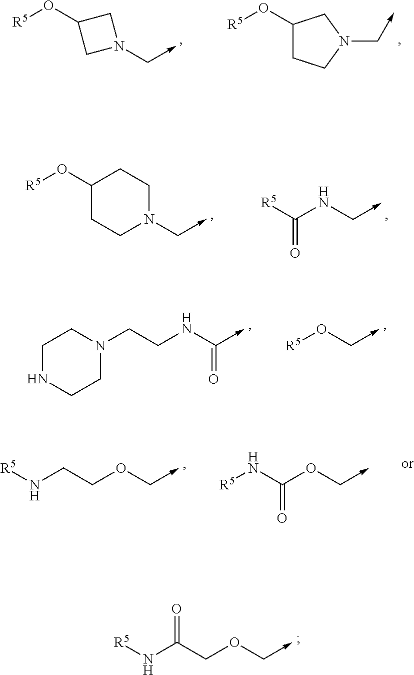

R⁵ is CH₃—(OC₂H₄)n-; and
n is 1 to 15.

Described below are a number of embodiments (E) of this first aspect of the invention, where for convenience E1 is identical thereto.
E1 A compound of formula (I) as defined above or a pharmaceutically acceptable salt thereof.
E2 A compound according to E1 or a pharmaceutically acceptable salt thereof wherein Het is ‘C-linked’ thiadiazolyl.
E3 A compound according to either E1 or E2 or a pharmaceutically acceptable salt thereof wherein Het is ‘C-linked’ 1,2,4 thiadiazolyl.
E4 A compound according to any of E1 to E3 or a pharmaceutically acceptable salt thereof wherein X is N.
E5 A compound according to any of E1 or E4 or a pharmaceutically acceptable salt thereof wherein R¹ is H.
E6 A compound according to any of E1 to E5 or a pharmaceutically acceptable salt thereof wherein R² is CN.
E7 A compound according to any of E1 to E6 or a pharmaceutically acceptable salt thereof wherein R^{3a} is CF₃ and R^{3b} is H.
E8 A compound according to any of E1 to E7 or a pharmaceutically acceptable salt thereof wherein R⁴ is

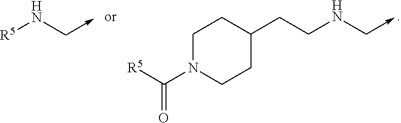

E9 A compound according to any of E1 to E8 or a pharmaceutically acceptable salt thereof wherein n is 3 to 12.
E10 A compound according to any of E1 to E9 or a pharmaceutically acceptable salt thereof wherein n is 4 to 12.
E1 A compound according to any of E1 to E10 or a pharmaceutically acceptable salt thereof wherein n is 4.
E12 A compound according to any of E1 to E10 or a pharmaceutically acceptable salt thereof wherein n is 12.
E13 A compound according to any of E1 to E7 or a pharmaceutically acceptable salt thereof wherein R⁴ is

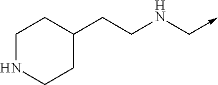

E14 The compound according to E1 or a pharmaceutically acceptable salt thereof that is:
4-({3-[2-({[2-(1-acetylpiperidin-4-yl)ethyl]amino}methyl)pyridin-4-yl]-3′-(trifluoromethyl) biphenyl-4-yl}oxy)-3-cyano-N-1,2,4-thiadiazol-5-ylbenzenesulfonamide;
5-chloro-2-fluoro-4-{[3-(2-{[(2-piperidin-4-ylethyl)amino]methyl}pyridin-4-yl)-3′-(trifluoromethyl)biphenyl-4-yl]oxy}-N-1,3-thiazol-4-ylbenzenesulfonamide;
6′-{2-cyano-4-[(1,2,4-thiadiazol-5-ylamino)sulfonyl]phenoxy}-N-(2-piperazin-1-ylethyl)-1,1′:3′,1″-terphenyl-3-carboxamide;
4-[(3″-{[4-(2-aminoethyl)piperazin-1-yl]methyl}-1,1′:3′,1″-terphenyl-4′-yl)oxy]-3-cyano-N-1,2,4-thiadiazol-5-ylbenzenesulfonamide;
3-cyano-4-[(3″-{[(2-piperidin-4-ylethyl)amino]methyl}-1,1′:3′,1″-terphenyl-4′-yl)oxy]-N-1,2,4-thiadiazol-5-ylbenzenesulfonamide;
5-chloro-2-fluoro-4-{[3-(2-{[(2-piperidin-4-ylethyl)amino]methyl}pyridin-4-yl)-3′-(trifluoromethyl)biphenyl-4-yl]oxy}-N-1,3,4-thiadiazol-2-ylbenzenesulfonamide;
4-({3-[2-(aminomethyl)pyridin-4-yl]-3′-(trifluoromethyl)biphenyl-4-yl}oxy)-5-chloro-2-fluoro-N-1,3,4-thiadiazol-2-ylbenzenesulfonamide;
2-[2-(2-methoxyethoxy)ethoxy]ethyl [(4-{4-[2-cyano-4-(1,2,4-thiadiazol-5-ylsulfamoyl) phenoxy]-3′-(trifluoromethyl)biphenyl-3-yl}pyridin-2-yl)methyl]carbamate;
3-cyano-4-((3-(2-(3-oxo-7,10,13,16-tetraoxa-2,4-diazaheptadecyl)pyridin-4-yl)-3′-(trifluoromethyl)-[1,1′-biphenyl]-4-yl)oxy)-N-(1,2,4-thiadiazol-5-yl)benzenesulfonamide;
4-((3-(2-(2,8,11,14,17-pentaoxa-5-azaoctadecyl)pyridin-4-yl)-3′-(trifluoromethyl)-[1,1′-biphenyl]-4-yl)oxy)-3-cyano-N-(1,2,4-thiadiazol-5-yl)benzenesulfonamide;
(4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methyl(2,5,8,11-tetraoxatridecan-13-yl)carbamate;
3-cyano-4-({3-[2-(2,5,8,11,14,17,20,23,26,29,32,35-dodecaoxa-38-azanonatriacontan-39-yl)pyridin-4-yl]-3′-(trifluoromethyl)biphenyl-4-yl}oxy)-N-(1,2,4-thiadiazol-5-yl)benzenesulfonamide;
3-cyano-4-({3-[2-(2,5,8,11,14-pentaoxapentadec-1-yl)pyridin-4-yl]-3′-(trifluoromethyl) biphenyl-4-yl}oxy)-N-(1,2,4-thiadiazol-5-yl)benzenesulfonamide;
2-((4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methoxy)-N-(2,5,8,11-tetraoxatridecan-13-yl)acetamide;
4-((3-(2-(5,8,11,14-tetraoxa-2-azapentadecyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-{[3″-({[2-(1-acetylpiperidin-4-yl)ethyl]amino}methyl)-1,1′:3′,1″-terphenyl-4′-yl]oxy}-3-cyano-N-1,2,4-thiadiazol-5-ylbenzenesulfonamide;
4-((3-(2-(((2-(1-(2,5,8,11,14,17,20,23,26,29,32,35-dodecaoxaoctatriacontan-38-oyl)piperidin-4-yl)ethyl)amino)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
3-cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide;
N-((4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methyl)-2,5,8,11,14,17,20,23,26,29,32,35-dodecaoxaoctatriacontan-38-amide;
4-((3-(2-((3-((2,5,8,11-tetraoxatridecan-13-yl)oxy)azetidin-1-yl)methyl)pyridin-4-yl)-3′-(trifluoromethyl)-[1,1′-biphenyl]-4-yl)oxy)-3-cyano-N-(2,4-di methoxybenzyl)-N-(1,2,4-thiadiazol-5-yl)benzenesulfonamide;
(R)-4-((3-(2-((3-((2,5,8,11-tetraoxatridecan-13-yl)oxy)pyrrolidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
(S)-4-((3-(2-((3-((2,5,8,11-tetraoxatridecan-13-yl)oxy)pyrrolidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-((3-(2-((4-((2,5,8,11-tetraoxatridecan-13-yl)oxy)piperidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-((3-(2-(4-(2,5,8,11,14,17,20,23,26,29,32,35-dodecaoxaoctatriacontan-38-oyl)piperazin-1-yl)pyridin-4-yl)-4′-(trifluoromethyl)-[1,1′-biphenyl]-4-yl)oxy)-3-cyano-N-(1,2,4-thiadiazol-5-yl)benzenesulfonamide
3-cyano-N-1,2,4-thiadiazol-5-yl-4-{[3-{2-[({2-[1-(trifluoroacetyl)piperidin-4-yl]ethyl}amino)methyl]pyridin-4-yl}-3′-(trifluoromethyl) biphenyl-4-yl]oxy}benzenesulfonamide;
4-({3-[2-(aminomethyl)pyridin-4-yl]-3′-(trifluoromethyl)biphenyl-4-yl}oxy)-3-cyano-N-1,2,4-thiadiazol-5-ylbenzenesulfonamide;
N-[(4-{4-[2-chloro-5-fluoro-4-(1,3,4-thiadiazol-2-ylsulfamoyl)phenoxy]-3′-(trifluoromethyl)biphenyl-3-yl}pyridin-2-yl)methyl]-2,5,8,11,14,17,20,23,26,29,32,35-dodecaoxaoctatriacontan-38-amide;
N-[(4-{4-[2-chloro-5-fluoro-4-(1,3-thiazol-4-ylsulfamoyl)phenoxy]-3′-(trifluoromethyl)biphenyl-3-yl}pyridin-2-yl)methyl]-2,5,8,11,14,17,20,23,26,29,32,35-dodecaoxaoctatriacontan-38-amide;
4-({3-[2-(aminomethyl)pyridin-4-yl]-3′-(trifluoromethyl)biphenyl-4-yl}oxy)-5-chloro-2-fluoro-N-(1,3-thiazol-4-yl)benzenesulfonamide;
5-chloro-2-fluoro-4-{[3-{2-[({2-[1-(38-oxo-2,5,8,11,14,17,20,23,26,29,32,35-dodecaoxaoctatriacontan-38-yl)piperidin-4-yl]ethyl}amino)methyl]pyridin-4-yl}-3′-(trifluoromethyl)biphenyl-4-yl]oxy}-N-(1,3-thiazol-4-yl)benzenesulfonamide;
3-cyano-4-({3″-[({2-[1-(38-oxo-2,5,8,11,14,17,20,23,26,29,32,35-dodecaoxaoctatriacontan-38-yl)piperidin-4-yl]ethyl}amino)methyl]-1,1′:3′,1″-terphenyl-4′-yl}oxy)-N-(1,2,4-thiadiazol-5-yl)benzenesulfonamide;
5-chloro-2-fluoro-4-{[3-{2-[4-(38-oxo-2,5,8,11,14,17,20,23,26,29,32,35-dodecaoxaoctatriacontan-38-yl)piperazin-1-yl]pyridin-4-yl}-4′-(trifluoromethyl)biphenyl-4-yl]oxy}-N-(1,3,4-thiadiazol-2-yl)benzenesulfonamide;
5-chloro-2-fluoro-4-{[3-{2-[({2-[1-(38-oxo-2,5,8,11,14,17,20,23,26,29,32,35-dodecaoxaoctatriacontan-38-yl)piperidin-4-yl]ethyl}amino)methyl]pyridin-4-yl}-3′-(trifluoromethyl)biphenyl-4-yl]oxy}-N-(1,3,4-thiadiazol-2-yl)benzenesulfonamide;
3-cyano-4-({3-[2-(2,5,8,11,14,17,20,23,26,29,32,35-dodecaoxa-38-azanonatriacontan-39-yl)pyridin-4-yl]-3′-(trifluoromethyl)biphenyl-4-yl}oxy)-N-(1,3,4-thiadiazol-2-yl)benzenesulfonamide; or
5-chloro-2-fluoro-N-(1,3-thiazol-4-yl)-4-{[3-{2-[({2-[1-(trifluoroacetyl) piperidin-4-yl]ethyl}amino)methyl]pyridin-4-yl}-3′-(trifluoromethyl) biphenyl-4-yl]oxy}benzenesulfonamide.
E15 The compound according to E1 or a pharmaceutically acceptable salt thereof that is:
3-cyano-4-({3-[2-(2,5,8,11,14,17,20,23,26,29,32,35-dodecaoxa-38-azanonatriacontan-39-yl)pyridin-4-yl]-3′-(trifluoromethyl)biphenyl-4-yl}oxy)-N-(1,2,4-thiadiazol-5-yl)benzenesulfonamide;
4-((3-(2-(5,8,11,14-tetraoxa-2-azapentadecyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-((3-(2-(((2-(1-(2,5,8,11,14,17,20,23,26,29,32,35-dodecaoxaoctatriacontan-38-oyl)piperidin-4-yl)ethyl)amino)methyl)pyridin-4-yl)-3′-(trifluoromethyl)-[1,1′-biphenyl]-4-yl)oxy)-3-cyano-N-(1,2,4-thiadiazol-5-yl)benzenesulfonamide; or
3-cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide.

The term ‘C-linked’ used in the definitions of formula (I) means that the group in question is joined via a ring carbon. The term ‘N-linked’ used in the definitions of formula (I) means that the group in question is joined via a ring nitrogen.

Hereinafter, all references to compounds of the invention include compounds of formula (I) or pharmaceutically acceptable salts, solvates, or multi-component complexes thereof, or pharmaceutically acceptable solvates or multi-component complexes of pharmaceutically acceptable salts of compounds of formula (I), as discussed in more detail below.

Preferred compounds of the invention are compounds of formula (I) or pharmaceutically acceptable salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

The skilled person will appreciate that the aforementioned salts include ones wherein the counterion is optically active, for example d-lactate or I-lysine, or racemic, for example dl-tartrate or dl-arginine.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of formula (I) or pharmaceutically acceptable salts thereof may exist in both unsolvated and solvated forms. The term ‘solvate’ is used herein to describe a molecular complex comprising a compound of formula (I) or a pharmaceutically acceptable salt thereof and one or more pharmaceutically acceptable solvent molecules, for example, ethanol. The term ‘hydrate’ is employed when said solvent is water. Pharmaceutically acceptable solvates in accordance with the invention include those wherein the solvent of crystallization may be isotopically substituted, e.g. D₂O, d₆-acetone and d₆-DMSO.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995), incorporated herein by reference. Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

Also included within the scope of the invention are multi-component complexes (other than salts and solvates) of compounds of formula (I) or pharmaceutically acceptable salts thereof wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004), incorporated herein by reference. For a general review of multi-component complexes, see J Pharm Sci, 64 (8), 1269-1288, by Haleblian (August 1975), incorporated herein by reference.

The compounds of the invention may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺⁾ or non-ionic (such as —N⁻N⁺(CH₃)₃) polar head group. For more information, see Crystals and the Polarizinq Microscope by N. H. Hartshorne and A. Stuart, 4^{th} Edition (Edward Arnold, 1970), incorporated herein by reference.

The compounds of the invention may be administered as prodrugs. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T Higuchi and W Stella) and ‘Bioreversible Carriers in Drug Design’, Pergamon Press, 1987 (ed. E B Roche, American Pharmaceutical Association).

Prodrugs can, for example, be produced by replacing appropriate functionalities present in a compound of formula (I) with certain moieties known to those skilled in the art as ‘pro-moieties’ as described, for example, in “Design of Prodrugs” by H Bundgaard (Elsevier, 1985).

Examples of prodrugs include phosphate prodrugs, such as dihydrogen or dialkyl (e.g. di-tert-butyl) phosphate prodrugs. Further examples of replacement groups in accordance with the foregoing examples and examples of other prodrug types may be found in the aforementioned references.

Also included within the scope of the invention are metabolites of compounds of formula (I), that is, compounds formed in vivo upon administration of the drug. Some examples of metabolites in accordance with the invention include, where the compound of formula (I) contains a phenyl (Ph) moiety, a phenol derivative thereof (-Ph>-PhOH);

Compounds of the invention containing one or more asymmetric carbon atoms can exist as two or more stereoisomers. Included within the scope of the invention are all stereoisomers of the compounds of the invention and mixtures of one or more thereof.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Mixtures of stereoisomers may be separated by conventional techniques known to those skilled in the art; see, for example, “Stereochemistry of Organic Compounds” by E. L. Eliel and S. H. Wilen (Wiley, New York, 1994.

The scope of the invention includes all crystal forms of the compounds of the invention, including racemates and racemic mixtures (conglomerates) thereof. Stereoisomeric conglomerates may also be separated by the conventional techniques described herein just above.

The scope of the invention includes all pharmaceutically acceptable isotopically-labelled compounds of the invention wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of the invention, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (I) can generally be prepared by conventional techniques known to those skilled in the art or by processes analogous to those described in the accompanying Examples and Preparations using an appropriate isotopically-labeled reagent in place of the non-labeled reagent previously employed.

Also within the scope of the invention are intermediate compounds as hereinafter defined, all salts, solvates and complexes thereof and all solvates and complexes of salts thereof as defined hereinbefore for compounds of formula (I). The invention includes all polymorphs of the aforementioned species and crystal habits thereof.

When preparing a compound of formula (I) in accordance with the invention, a person skilled in the art may routinely select the form of intermediate which provides the best combination of features for this purpose. Such features include the melting point, solubility, processability and yield of the intermediate form and the resulting ease with which the product may be purified on isolation.

The compounds of the invention may be prepared by any method known in the art for the preparation of compounds of analogous structure. In particular, the compounds of the invention can be prepared by the procedures described in the Schemes that follow, or by the specific methods described in the Examples, or by processes similar to either.

The skilled person will appreciate that the experimental conditions set forth in the schemes that follow are illustrative of suitable conditions for effecting the transformations shown and that it may be necessary or desirable to vary the precise conditions employed for the preparation of compounds of formula (I).

In addition, the skilled person will appreciate that it may be necessary or desirable at any stage in the synthesis of compounds of the invention to protect one or more sensitive groups, so as to prevent undesirable side reactions. In particular, it may be necessary or desirable to protect amino groups. The protecting groups used in the preparation of the compounds of the invention may be used in conventional manner.

See, for example, those described in ‘Greene's Protective Groups in Organic Synthesis’ by Theodora W Greene and Peter G M Wuts, fourth edition, (John Wiley and Sons, 2006), in particular chapter 7 (“Protection for the Amino Group”), incorporated herein by reference, which also describes methods for the removal of such groups.

With particular reference to the schemes that follow the skilled person will appreciate that it may be desirable to protect:
the sulphonamide —NH— group in precursor compounds to formula (I), such as those of formula (II), e.g. with dimethoxybenzyl or tertbutoxycarbonyl; convenient conditions for deprotection are described in Scheme 1, process step (ii);
an R⁴¹ containing amino group in the amines of formula (V); convenient protecting groups, and their removal, are those described for the ‘additional or alternative’ deprotection conditions in Scheme 1.

Unless stated otherwise, in the following processes R¹, R², R^{3a}, R^{3b}, R⁴ and Het are as previously defined for a compound of formula (I). Lg is a suitable leaving group, such as halo (e.g. Br) or a sulphonate ester (e.g mesylate, triflate or tosylate). M is an optionally substituted/ligated metal or boron group suitable for cross coupling reactions, such as trialkylstannane, dihydroxyborane, dialkoxyborane or halozinc. Where ratios of solvents are given, the ratios are by volume. Where the following reactions require heating, this may be effected thermally or by microwave irradiation.

According to a first process, compounds of formula (I) may be prepared from compounds of formulae (II) and (III), as illustrated by Scheme 1.

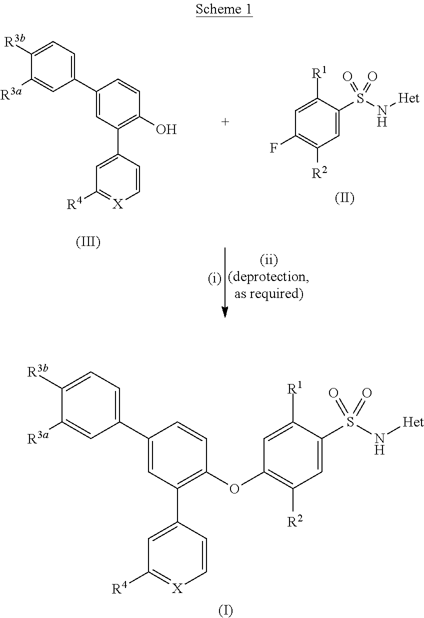

Compounds of formula (I) may be prepared from compounds of formula (II) and (III) according to process step (i), a nucleophilic aromatic substitution reaction followed by, if necessary, process step (ii), a deprotection reaction.

Convenient conditions comprise:
process step (i): an inorganic base in an organic solvent, at either room or elevated temperatures; followed by
process step (ii): acid mediated deprotection.

Preferred conditions comprise:
process step (i): potassium carbonate or potassium phosphate in DMSO or DMF, at from room temperature to 90° C.; followed by
process step (ii): 4M HCl in dioxane or TFA in DCM at room temperature.

The skilled person will further appreciate that it may be desirable to protect, where present, an R⁴ amino group in a compound of formula (III), and hence to employ additional or alternative deprotection conditions. Conveniently, where the R⁴ protecting group is:
benzyloxycarbonyl, preferred deprotection conditions comprise hydrogen bromide in acetic acid at from 50° C. to room temperature;
2,2,2-trichloroethyloxycarbonyl, preferred deprotection conditions comprise zinc dust in acetic acid at room temperature;
trilfluoroacetyl, preferred deprotection conditions comprise 7M ammonia in MeOH at room temperature, or aqueous sodium carbonate solution at reflux;
tert-butoxycarbonyl, preferred deprotection conditions comprise 4M HCl in dioxane, or TFA in DCM.

According to a second process, compounds of formula (I) wherein R⁴ is

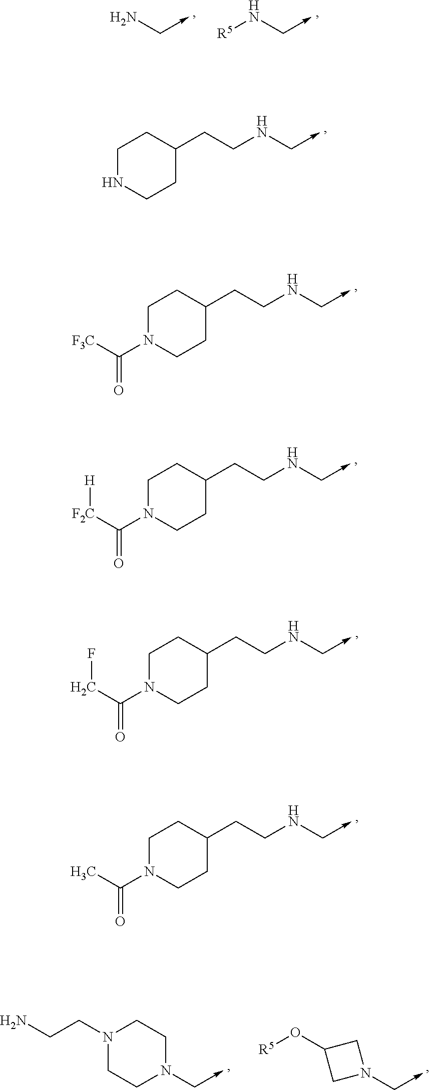

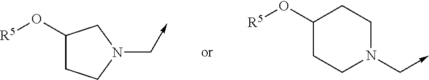

(hereinafter R⁴ⁱ), may be prepared from compounds of formulae (IV) and (V), as illustrated by Scheme 2.

Compounds of formula (I) may be prepared from compounds of formula (IV) according to process step (iii), a reductive amination step with amines of formula (V) followed by, if necessary, process step (ii), a deprotection reaction.

The skilled person will appreciate that for a given R⁴ⁱ the amine of formula (V), R⁴¹NH₂, is the corresponding ‘terminal des-methylene’ derivative. For example, where:
R⁴ⁱ is H₂N—CH₂→, the amine of formula (V) is NH₃;
R⁴ⁱ is R⁵HN—CH₂→, the amine of formula (V) is R⁵NH₂; and so on.

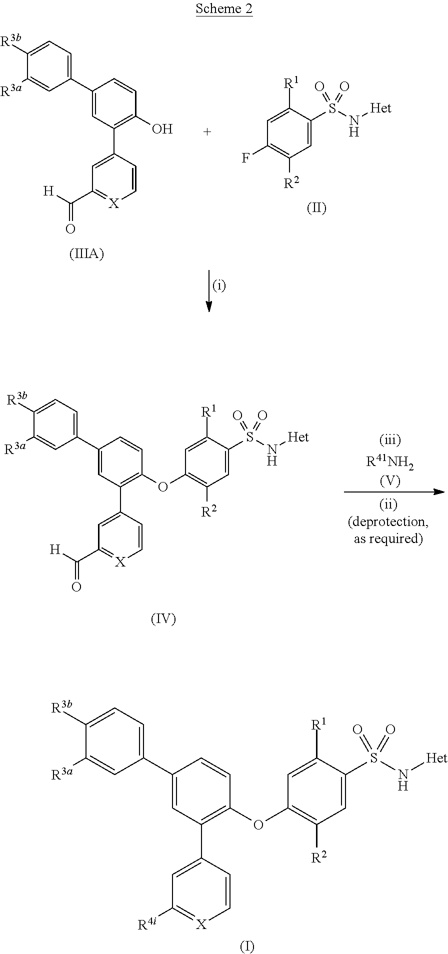

Preferred conditions comprise reductive amination with sodium triacetoxyborohydride in acetic acid at room temperature, followed by deprotection according to the conditions described in Scheme 1, process step (ii).

Compounds of formula (IV) may be prepared from compounds of formula (II) and (IIIA) under the nucleophilic aromatic substitution reaction conditions described in Scheme 1, process step (i).

According to a third process, compounds of formula (I) wherein R⁴ is R⁴ⁱ may be prepared from compounds of formulae (V) and (VI) as illustrated by Scheme 3 that follows.

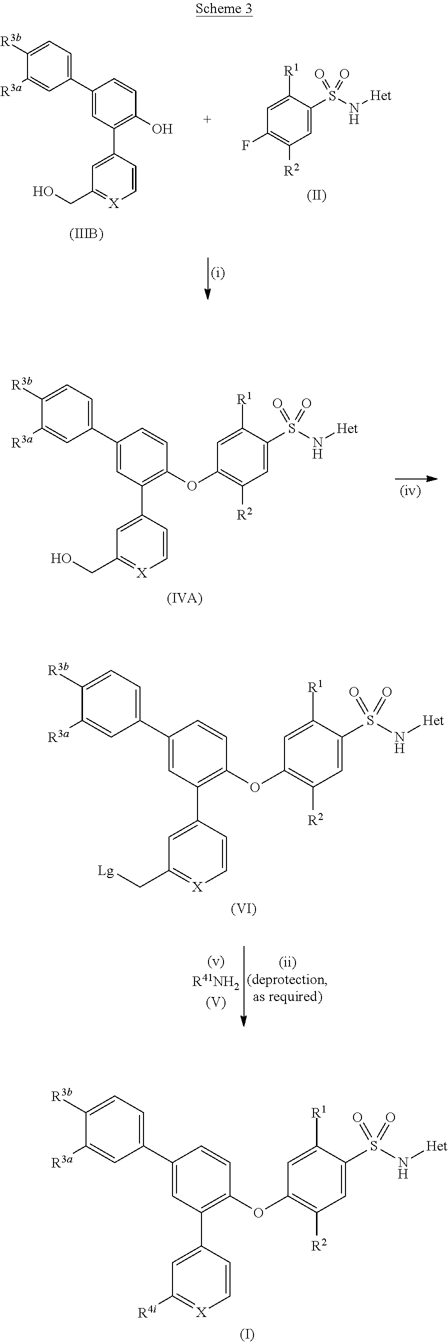

Compounds of formula (I) may be prepared from compounds of formula (VI) according to process step (v), an alkylation step followed by, if necessary, process step (ii), a deprotection reaction.

Preferred conditions comprise alkylation in the presence of DIPEA in DCM at room temperature, followed by deprotection if necessary according to the conditions described in Scheme 1, process step (ii).

Compounds of formula (VI) may be prepared from compounds of formula (IVA), according to process step (iv), a conversion of an alcohol into a leaving group through reaction with Lg-Cl. Preferred conditions comprise mesyl chloride with DIPEA in DCM at room temperature.

Compounds of formula (IVA) may be prepared from compounds of formula (II) and (IIIB) under the nucleophilic aromatic substitution reaction conditions described in Scheme 1, process step (i).

According to a fourth process, compounds of formula (I) wherein R⁴ is

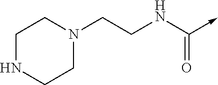

may be prepared from compounds of formulae (VA) and (VII) as illustrated by Scheme 4 that follows.

Compounds of formula (I) may be prepared from compounds of formula (VII) and (VA) according to process step (vi), an amide bond formation reaction followed by, if necessary, process step (ii), a deprotection reaction.

Conveniently amide bond formation reactions include a suitable acid activating group in combination with an inorganic base. Preferred conditions comprise amide bond formation in the presence of carbonyldiimidazole or COMU®, DIPEA, in DMF and at room temperature; followed by deprotection if necessary according to the conditions described in Scheme 1, process step (i).

Compounds of formula (VII) may be prepared from compounds of formulae (VIII) and (IX) according to process step (vii), a Suzuki cross-coupling reaction. Typical conditions employ a palladium catalyst with a suitable phosphorus ligand, an inorganic base and elevated temperatures. Preferred conditions comprise Pd(dppf)C₂ with sodium carbonate in DMF at 150° C.

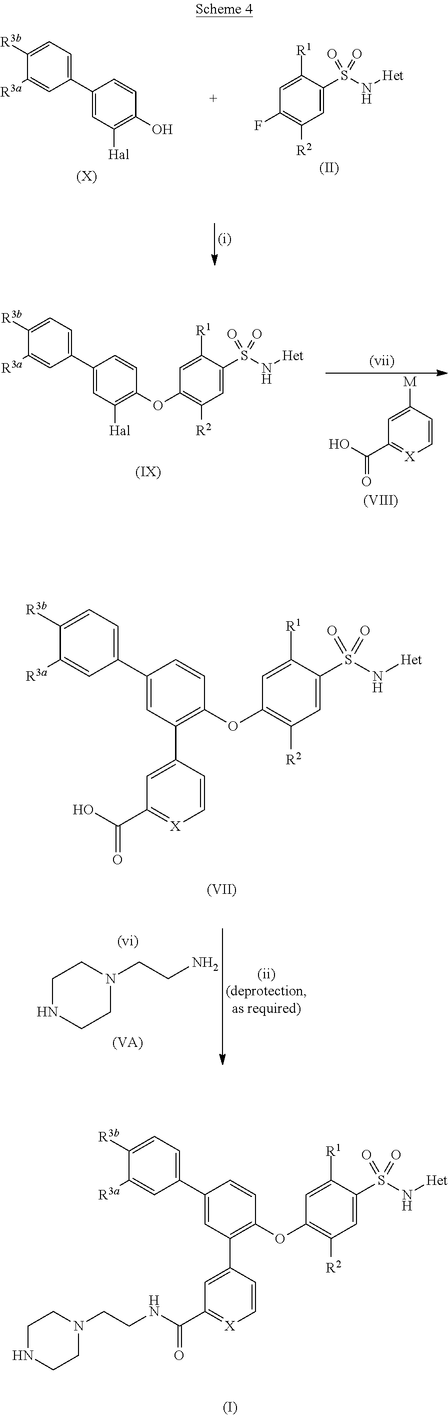

Compounds of formula (IX) may be prepared from compounds of formula (II) and (X) under the nucleophilic aromatic substitution reaction conditions described in Scheme 1, process step (i).

According to a fifth process, compounds of formula (I) wherein R⁴ is

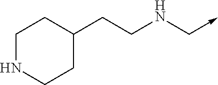

may be prepared from compounds of formulae (XI) and (XII) as illustrated by Scheme 5 that follows, wherein Pg is dimethoxybenzyl or tertbutoxycarbonyl.

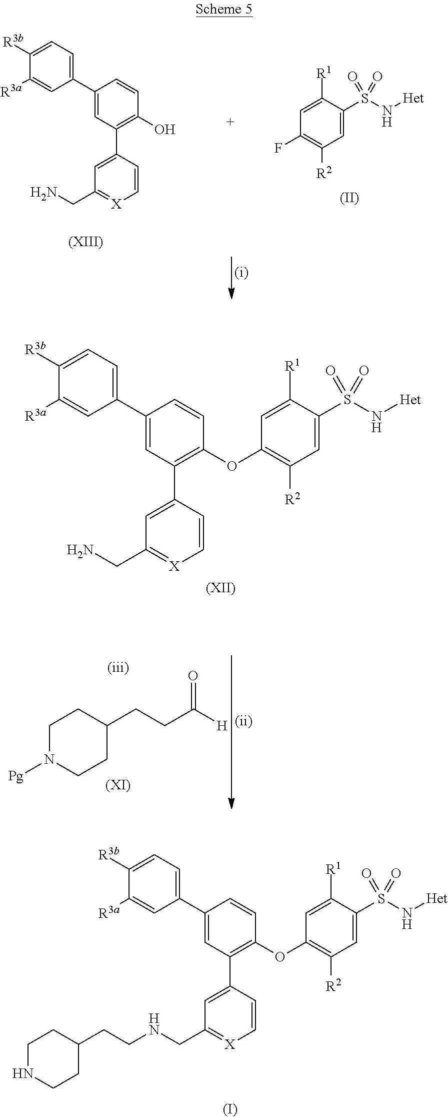

Compounds of formula (I) may be prepared from compounds of formulae (XI) and (XII) according to process step (iii), a reductive amination step followed byprocess step (ii), a deprotection reaction. Convenient conditions for each step are respectively described in Scheme 2 step (iii) and Scheme 1 step (ii).

Compounds of formula (XII) may be prepared from compounds of formulae (XIII) and (II) according to process step (i), as described in Scheme 1.

According to a sixth process, compounds of formula (I) wherein R⁴ is either:

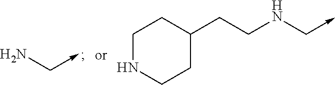

(hereinafter R⁴ⁱⁱ); may be converted into the corresponding compounds of formula (I) wherein R⁴ is, respectively,

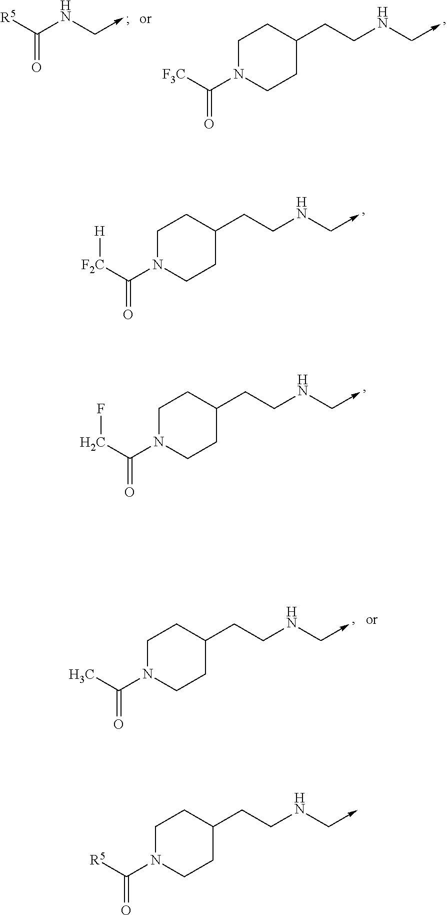

(hereinafter R⁴ⁱⁱⁱ); by acylation with the corresponding compound of formula (XVI), as illustrated by Scheme 6 that follows.

Preferably the interconversion is carried out in a suitable organic solvent such as DCM or DMF, optionally in the presence of triethylamine, and at room temperature.

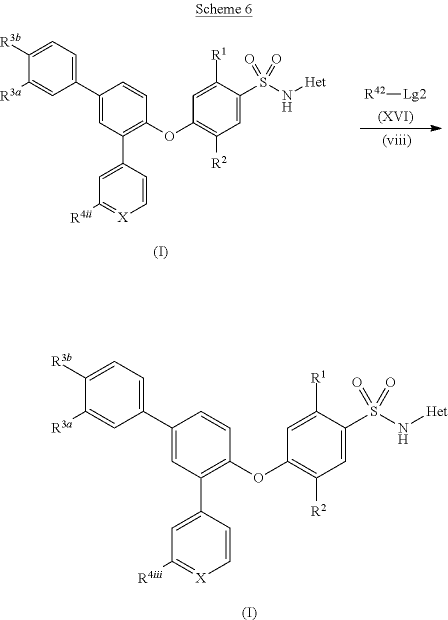

wherein Lg2 is a suitable leaving group such as N-hydroxysuccinimide, para-nitrophenol or an anhydride.

Compounds of formulae (II), (III), (IIIA), (IIIB), (V), (VA), (VIII), (X), (XI), (XIII) and (XVI) are commercially available, known from the literature, easily prepared by methods well known to those skilled in the art, or can be made according to preparations described herein.

All new processes for preparing compounds of formula (I), and corresponding new intermediates employed in such processes, form further aspects of the present invention.

Compounds of the invention intended for pharmaceutical use may be administered as crystalline or amorphous products or may exist in a continuum of solid states ranging from fully amorphous to fully crystalline. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

In another aspect the invention provides a pharmaceutical composition comprising a compound of the invention together with one or more pharmaceutically acceptable excipients.

Pharmaceutical compositions suitable for the delivery of compounds of the present invention and methods for their preparation will be readily apparent to those skilled in the art. Such compositions and methods for their preparation may be found, for example, in “Remington's Pharmaceutical Sciences”, 19th Edition (Mack Publishing Company, 1995).

Suitable modes of administration include oral, parenteral, topical, inhaled/intranasal, rectal/intravaginal, and ocular/aural administration.

Formulations suitable for the aforementioned modes of administration may be formulated to be immediate and/or modified release. Modified release formulations include delayed-, sustained-, pulsed-, controlled-, targeted and programmed release.

The compounds of the invention may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays, liquid formulations and buccal/mucoadhesive patches.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant. Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. The formulation of tablets is discussed in “Pharmaceutical Dosage Forms: Tablets”, Vol. 1, by H. Lieberman and L. Lachman (Marcel Dekker, New York, 1980).

Suitable modified release formulations for the purposes of the invention are described in U.S. Pat. No. 6,106,864. Details of other suitable release technologies such as high energy dispersions and osmotic and coated particles are to be found in “Pharmaceutical Technology On-line”, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (I) used in the preparation of parenteral solutions may be increased by the use of appropriate formulation techniques, such as the incorporation of solubility-enhancing agents. Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as I-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

In the case of dry powder inhalers and aerosols, the dosage unit is determined by means of a valve which delivers a metered amount. Units in accordance with the invention are typically arranged to administer a metered dose or “puff” containing from 1 μg to 100 mg of the compound of formula (I). The overall daily dose will typically be in the range 1 μg to 200 mg which may be administered in a single dose or, more usually, as divided doses throughout the day.

The compounds of the invention may be administered rectally or vaginally, for example, in the form of a suppository, pessary, microbicide, vaginal ring or enema. Cocoa butter is a traditional suppository base, but various alternatives may be used as appropriate.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

For administration to human patients, the total daily dose of the compounds of the invention is typically in the range 1 mg to 10 g, such as 10 mg to 1 g, for example 25 mg to 500 mg depending, of course, on the mode of administration and efficacy. For example, oral administration may require a total daily dose of from 50 mg to 100 mg. The total daily dose may be administered in single or divided doses and may, at the physician's discretion, fall outside of the typical range given herein. These dosages are based on an average human subject having a weight of about 60 kg to 70 kg. The physician will readily be able to determine doses for subjects whose weight falls outside this range, such as infants and the elderly.

As noted above, the compounds of the invention are useful because they exhibit pharmacological activity in animals, i.e., Nav1.7 channel inhibition. More particularly, the compounds of the invention are of use in the treatment of disorders for which a Nav1.7 inhibitor is indicated. Preferably the animal is a mammal, more preferably a human.

In a further aspect of the invention there is provided a compound of the invention for use as a medicament.

In a further aspect of the invention there is provided a compound of the invention for the treatment of a disorder for which a Nav1.7 inhibitor is indicated.

In a further aspect of the invention there is provided use of a compound of the invention for the preparation of a medicament for the treatment of a disorder for which a Nav1.7 inhibitor is indicated.

In a further aspect of the invention there is provided a method of treating a disorder in an animal (preferably a mammal, more preferably a human) for which a Nav1.7 inhibitor is indicated, comprising administering to said animal a therapeutically effective amount of a compound of the invention.

Disorders for which a Nav1.7 inhibitor is indicated include pain. Pain may be either acute or chronic and additionally may be of central and/or peripheral origin. Pain may be of a neuropathic and/or nociceptive and/or inflammatory nature, such as pain affecting either the somatic or visceral systems, as well as dysfunctional pain affecting multiple systems.

Physiological pain is an important protective mechanism designed to warn of danger from potentially injurious stimuli from the external environment. The system operates through a specific set of primary sensory neurones and is activated by noxious stimuli via peripheral transducing mechanisms (see Meyer et al., 2006, Wall and Melzack's Textbook of Pain (5^{th} Ed), Chapterl). These sensory fibres are known as nociceptors, and are characteristically small diameter axons with slow conduction velocities, of which there are two main types, A-delta fibres (myelinated) and C fibres (non-myelinated). Nociceptors encode the intensity, duration and quality of noxious stimulus and by virtue of their topographically organised projection to the spinal cord, the location of the stimulus. The activity generated by nociceptor input is transferred, after complex processing in the dorsal horn, either directly, or via brain stem relay nuclei, to the ventrobasal thalamus and then on to the cortex, where the sensation of pain is generated.

Pain may generally be classified as acute or chronic. Acute pain begins suddenly and is short-lived (usually twelve weeks or less). It is usually, although not always, associated with a specific cause such as a defined injury, is often sharp and severe and can result from numerous origins such as surgery, dental work, a strain or a sprain. Acute pain does not generally result in any persistent psychological response. When a substantial injury occurs to body tissue, via disease or trauma, the characteristics of nociceptor activation may be altered such that there is sensitisation in the periphery, locally around the injury and centrally where the nociceptors terminate. These effects lead to a hightened sensation of pain. In acute pain these mechanisms can be useful, in promoting protective behaviours which may better enable repair processes to take place. The normal expectation would be that sensitivity returns to normal once the injury has healed. However, in many chronic pain states, the hypersensitivity far outlasts the healing process and is often due to nervous system injury or alteration which can be associated with maladaptation and aberrant activity (Woolf & Salter, 2000, Science, 288, 1765-1768). As such, chronic pain is long-term pain, typically persisting for more than three months and leading to significant psychological and emotional problems. Common examples of chronic pain are neuropathic pain (e.g. painful diabetic neuropathy or postherpetic neuralgia), carpal tunnel syndrome, back pain, headache, cancer pain, arthritic pain and chronic post-surgical pain, but may include any chronic painful condition affecting any system, such as those described by the International Association for the Study of Pain (Classification of Chronic Pain, a publication freely available for download at http://www.iasp-pain.org).

The clinical manifestation of pain is present when discomfort and abnormal sensitivity feature among the patient's symptoms. Patients tend to be quite heterogeneous and may present with various pain symptoms. Such symptoms can include: 1) spontaneous pain which may be dull, burning, or stabbing; 2) exaggerated pain responses to noxious stimuli (hyperalgesia); and 3) pain produced by normally innocuous stimuli (allodynia) (Meyer et al., 2006, Wall and Melzack's Textbook of Pain (5^{th} Ed), Chapterl). Although patients suffering from various forms of acute and chronic pain may have similar symptoms, the underlying mechanisms may be different and may, therefore, require different treatment strategies. Apart from acute or chronic, pain can also be broadly categorized into: nociceptive pain, affecting either the somatic or visceral systems, which can be inflammatory in nature (associated with tissue damage and the infiltration of immune cells); or neuropathic pain.

Nociceptive pain can be defined as the process by which intense thermal, mechanical, or chemical stimuli are detected by a subpopulation of peripheral nerve fibers, called nociceptors, and can be induced by tissue injury or by intense stimuli with the potential to cause injury. Pain afferents are activated by transduction of stimuli by nociceptors at the site of injury and activate neurons in the spinal cord at the level of their termination. This is then relayed up the spinal tracts to the brain where pain is perceived (Meyer et al., 2006, Wall and Melzack's Textbook of Pain (5^{th} Ed), Chapterl). Myelinated A-delta fibres transmit rapidly and are responsible for sharp and stabbing pain sensations, whilst unmyelinated C fibres transmit at a slower rate and convey a dull or aching pain. Moderate to severe acute nociceptive pain is a prominent feature of pain from strains/sprains, burns, myocardial infarction and acute pancreatitis, post-operative pain (pain following any type of surgical procedure), posttraumatic pain, pain associated with gout, cancer pain and back pain. Cancer pain may be chronic pain such as tumour related pain (e.g. bone pain, headache, facial pain or visceral pain) or pain associated with cancer therapy (e.g. in response to chemotherapy, immunotherapy, hormonal therapy or radiotherapy). Back pain may be due to herniated or ruptured intervertabral discs or abnormalities of the lumber facet joints, sacroiliac joints, paraspinal muscles or the posterior longitudinal ligament. Back pain may resolve naturally but in some patients, where it lasts over 12 weeks, it becomes a chronic condition which can be particularly debilitating.

Nociceptive pain can also be related to inflammatory states. The inflammatory process is a complex series of biochemical and cellular events, activated in response to tissue injury or the presence of foreign substances, which results in swelling and pain (McMahon et al., 2006, Wall and Melzack's Textbook of Pain (5^{th} Ed), Chapter3). A common inflammatory condition associated with pain is arthritis. It has been estimated that almost 27 million Americans have symptomatic osteoarthritis (OA) or degenerative joint disease (Lawrence et al., 2008, Arthritis Rheum, 58, 15-35); most patients with osteoarthritis seek medical attention because of the associated pain. Arthritis has a significant impact on psychosocial and physical function and is known to be the leading cause of disability in later life. Rheumatoid arthritis is an immune-mediated, chronic, inflammatory polyarthritis disease, mainly affecting peripheral synovial joints. It is one of the commonest chronic inflammatory conditions in developed countries and is a major cause of pain.

In regard to nociceptive pain of visceral origin, visceral pain results from the activation of nociceptors of the thoracic, pelvic, or abdominal organs (Bielefeldt and Gebhart, 2006, Wall and Melzack's Textbook of Pain (5^{th} Ed), Chapter48). This includes the reproductive organs, spleen, liver, gastrointestinal and urinary tracts, airway structures, cardiovascular system and other organs contained within the abdominal cavity. As such visceral pain refers to pain associated with conditions of such organs, such as painful bladder syndrome, interstitial cystitis, prostatitis, ulcerative colitis, Crohn's disease, renal colic, irritable bowl syndrome, endometriosis and dysmenorrheal (Classification of Chronic Pain, available at http://www.iasp-pain.org). Currently the potential for a neuropathic contribution (either through central changes or nerve injury/damage) to visceral pain states is poorly understood but may play a role in certain conditions (Aziz et al., 2009, Dig Dis 27, Suppl 1, 31-41)

Neuropathic pain is currently defined as pain arising as a direct consequence of a lesion or disease affecting the somatosensory system. Nerve damage can be caused by trauma and disease and thus the term ‘neuropathic pain’ encompasses many disorders with diverse aetiologies. These include, but are not limited to, peripheral neuropathy, diabetic neuropathy, post herpetic neuralgia, trigeminal neuralgia, back pain, cancer neuropathy, HIV neuropathy, phantom limb pain, carpal tunnel syndrome, central post-stroke pain and pain associated with chronic alcoholism, hypothyroidism, uremia, multiple sclerosis, spinal cord injury, Parkinson's disease, epilepsy and vitamin deficiency. Neuropathic pain is pathological as it has no protective role. It is often present well after the original cause has dissipated, commonly lasting for years, significantly decreasing a patient's quality of life (Dworkin, 2009, Am J Med, 122, S1-S2; Geber et al., 2009, Am J Med, 122, S3-S12; Haanpaa et al., 2009, Am J Med, 122, S13-S21). The symptoms of neuropathic pain are difficult to treat, as they are often heterogeneous even between patients with the same disease (Dworkin, 2009, Am J Med, 122, S1-S2; Geber et al., 2009, Am J Med, 122, S3-S12; Haanpaa et al., 2009, Am J Med, 122, S13-S21). They include spontaneous pain, which can be continuous, and paroxysmal or abnormal evoked pain, such as hyperalgesia (increased sensitivity to a noxious stimulus) and allodynia (sensitivity to a normally innocuous stimulus).

It should be noted that some types of pain have multiple aetiologies and thus can be classified in more than one area, e.g. back pain, cancer pain and even migraine headaches may include both nociceptive and neuropathic components.

Similarly other types of chronic pain, perhaps less well understood, are not easily defined by the simplistic definitions of nociceptive or neuropathic. Such conditions include in particular fibromyalgia and chronic regional pain syndrome, which are often described as dysfunctional pain states e.g. fibromyalgia or complex regional pain syndrome (Woolf, 2010, J Clin Invest, 120, 3742-3744), but which are included in classifications of chronic pain states (Classification of Chronic Pain, available at http://www.iasp-pain.org).

A Nav1.7 inhibitor may be usefully combined with another pharmacologically active compound, or with two or more other pharmacologically active compounds, particularly in the treatment of pain. Such combinations offer the possibility of significant advantages, including patient compliance, ease of dosing and synergistic activity.

In the combinations that follow the compound of the invention may be administered simultaneously, sequentially or separately in combination with the other therapeutic agent or agents.

A Nav1.7 inhibitor of formula (I), or a pharmaceutically acceptable salt thereof, as defined above, may be administered in combination with one or more agents selected from:
a selective Nav1.3 channel modulator, such as a compound disclosed in WO2008/118758;
a selective Nav1.8 channel modulator, such as a compound disclosed in WO2013/114250;
a selective Nav1.9 channel modulator;
a compound which modulates activity at more than one Nav channel, including a non-selective modulator such as bupivacaine, carbamazepine, lamotrigine, lidocaine, mexiletine or phenytoin;
any inhibitor of nerve growth factor (NGF) signaling, such as: an agent that binds to NGF and inhibits NGF biological activity and/or downstream pathway(s) mediated by NGF signaling (e.g. tanezumab), a TrkA antagonist or a p75 antagoinsist, or an agent that inhibits downstream signaling in regard to NGF stimulated TrkA or P75 signalling;
an inhibitor of neurotrophic pathways, where such inhibition is achieved by: (a) an agent that binds to nerve growth factor (NGF) (e.g. tanezumab, fasinumab or fulranumab), brain-derived neurotrophic factor (BDNF), neurotrophin-3 (NT-3) or neurotrophin-4 (NT-4), or to more than one of the aforementioned neurotrophins (e.g. soluble P75); or (b) an agent that inhibits receptor function at one or more of TrKA, TrKB, TrKC or P75, either at the orthosteric site, an allosteric site or by inhibition of the catalytic activity of the receptor(s);
a compound which increases the levels of endocannabinoid, such as a compound with fatty acid amid hydrolase inhibitory (FAAH) or monoacylglycerol lipase (MAGL) activity;
an analgesic, in particular paracetamol;
an opioid analgesic, such as: buprenorphine, butorphanol, cocaine, codeine, dihydrocodeine, fentanyl, heroin, hydrocodone, hydromorphone, levallorphan levorphanol, meperidine, methadone, morphine, nalmefene, nalorphine, naloxone, naltrexone, nalbuphine, oxycodone, oxymorphone, propoxyphene or pentazocine;
an opioid analgesic which preferentially stimulates a specific intracellular pathway, for example G-protein as opposed to beta arrestin recruitment, such as TRV130; an opioid analgesic with additional pharmacology, such as: noradrenaline (norepinephrine) reuptake inhibitory (NRI) activity, e.g. tapentadol; serotonin and norepinephrine reuptake inhibitory (SNRI) activity, e.g. tramadol; or nociceptin receptor (NOP) agonist activity, such as GRT6005;
a nonsteroidal antiinflammatory drug (NSAID), such as a non-selective cyclooxygenase (COX) inhibitor, e.g. aspirin, diclofenac, diflusinal, etodolac, fenbufen, fenoprofen, flufenisal, flurbiprofen, ibuprofen, indomethacin, ketoprofen, ketorolac, meclofenamic acid, mefenamic acid, meloxicam, nabumetone, naproxen, nimesulide, nitroflurbiprofen, olsalazine, oxaprozin, phenylbutazone, piroxicam, sulfasalazine, sulindac, tolmetin or zomepirac; or a COX-2 selective inhibitor, e.g. celecoxib, deracoxib, etoricoxib, mavacoxib or parecoxib;
a prostaglandin E₂ subtype 4 (EP4) antagonist;
a microsomal prostaglandin E synthase type 1 (mPGES-1) inhibitor;
a sedative, such as glutethimide, meprobamate, methaqualone or dichloralphenazone;
a GABA_{A} modulator with broad subtype modulatory effects mediated via the benzodiazepine binding site, such as chlordiazepoxide, alprazolam, diazepam, lorazepam, oxazepam, temazepam, triazolam, clonazepam or clobazam;
a GABA_{A} modulator with subtype-selective modulatory effects mediated via the benzodiazepine binding site with reduced adverse effects, for example sedation, such as TPA023, TPA023B, L-838,417, CTP354 or NSD72;
a GABA_{A} modulator acting via alternative binding sites on the receptor, such as barbiturates, e.g. amobarbital, aprobarbital, butabital, mephobarbital, methohexital, pentobarbital, phenobartital, secobarbital, or thiopental; neurosteroids such as alphaxalone, alphadolone or ganaxolone; □-subunit ligands, such as etifoxine; or □-preferring ligands, such as gaboxadol;
a GlyR3 agonist or positive allosteric modulator;
a skeletal muscle relaxant, e.g. baclofen, carisoprodol, chlorzoxazone, cyclobenzaprine, metaxolone, methocarbamol or orphrenadine;
a glutamate receptor antagonist or negative allosteric modulator, such as an NMDA receptor antagonist, e.g. dextromethorphan, dextrorphan, ketamine or, memantine; or an mGluR antagonist or modulator;
an alpha-adrenergic, such as clonidine, guanfacine or dexmetatomidine;
a beta-adrenergic such as propranolol;
a tricyclic antidepressant, e.g. desipramine, imipramine, amitriptyline or nortriptyline;
a tachykinin (NK) antagonist, such as aprepitant or maropitant;
a muscarinic antagonist, e.g oxybutynin, tolterodine, propiverine, tropsium chloride, darifenacin, solifenacin, temiverine and ipratropium;
a cholinergic (nicotinic) analgesic, such as ispronicline (TC-1734), varenicline or nicotine;
a Transient Receptor Potential V1 (TRPV1) receptor agonist (e.g. resinferatoxin or capsaicin) or antagonist (e.g. capsazepine or mavatrap);
a Transient Receptor Potential A1 (TRPA1) receptor agonist (e.g. cinnamaldehyde or mustard oil) or antagonist (e.g. GRC17536 or CB-625);
a Transient Receptor Potential M8 (TRPM8) receptor agonist (e.g. menthol or icilin) or antagonist;
a Transient Receptor Potential V3 (TRPV3) receptor agonist or antagonist (e.g. GRC-15300);
a corticosteroid such as dexamethasone;
a 5-HT receptor agonist or antagonist, particularly a 5-HT_{1B/1D} agonist, such as eletriptan, sumatriptan, naratriptan, zolmitriptan or rizatriptan;
a 5-HT_{2A} receptor antagonist;
a PDEV inhibitor, such sildenafil, tadalafil or vardenafil;
an alpha-2-delta ligand such as gabapentin, gabapentin enacarbil or pregabalin;
a serotonin reuptake inhibitor (SRI) such as sertraline, demethylsertraline, fluoxetine, norfluoxetine, fluvoxamine, paroxetine, citalopram, desmethylcitalopram, escitalopram, d,l-fenfluramine, femoxetine, ifoxetine, cyanodothiepin, litoxetine, dapoxetine, nefazodone, cericlamine and trazodone;
anNRI, such as maprotiline, lofepramine, mirtazepine, oxaprotiline, fezolamine, tomoxetine, mianserin, buproprion, buproprion metabolite hydroxybuproprion, nomifensine and viloxazine, especially a selective noradrenaline reuptake inhibitor such as reboxetine;
an SNRI, such as venlafaxine, O-desmethylvenlafaxine, clomipramine, desmethylclomipramine, duloxetine, milnacipran and imipramine;
an inducible nitric oxide synthase (iNOS) inhibitor;
a leukotriene B4 antagonist;
a 5-lipoxygenase inhibitor, such as zileuton;
a potassium channel opener or positive modulator, such as an opener or positive modulator of KCNQ/Kv7 (e.g. retigabine or flupirtine), a G protein-coupled inwardly-rectifying potassium channel (GIRK), a calcium-activated potassium channel (Kca) or a potassium voltage-gated channel such as a member of subfamily A (e.g. Kv1.1), subfamily B (e.g. Kv2.2) or subfamily K (e.g. TASK, TREK or TRESK);
a P2X₃ receptor antagonist (e.g. AF219) or an antagonist of a receptor which contains as one of its subunits the P2X₃ subunit, such as a P2X_{2/3} heteromeric receptor;
a Caᵥ2.2 calcium channel blocker (N-type), such as ziconotide; and
a Caᵥ3.2 calcium channel blocker (T-type), such as ethosuximide.

There is also included within the scope the present invention combinations of a compound of the invention together with one or more additional therapeutic agents which slow down the rate of metabolism of the compound of the invention, thereby leading to increased exposure in patients. Increasing the exposure in such a manner is known as boosting. This has the benefit of increasing the efficacy of the compound of the invention or reducing the dose required to achieve the same efficacy as an unboosted dose. The metabolism of the compounds of the invention includes oxidative processes carried out by P450 (CYP450) enzymes, particularly CYP 3A4 and conjugation by UDP glucuronosyl transferase and sulphating enzymes. Thus, among the agents that may be used to increase the exposure of a patient to a compound of the present invention are those that can act as inhibitors of at least one isoform of the cytochrome P450 (CYP450) enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include ritonavir, saquinavir, ketoconazole, N-(3,4-difluorobenzyl)-N-methyl-2-{[(4-methoxypyridin-3-yl)amino]sulfonyl}benzamide and N-(1-(2-(5-(4-fluorobenzyl)-3-(pyridin-4-yl)-1H-pyrazol-1-yl)acetyl) piperidin-4-yl)methanesulfonamide.

It is within the scope of the invention that two or more pharmaceutical compositions, at least one of which contains a compound of the invention, may conveniently be combined in the form of a kit suitable for coadministration of the compositions. Thus the kit of the invention comprises two or more separate pharmaceutical compositions, at least one of which contains a compound of the invention, and means for separately retaining said compositions, such as a container, divided bottle, or divided foil packet. An example of such a kit is the familiar blister pack used for the packaging of tablets, capsules and the like. The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

In another aspect the invention provides a pharmaceutical product (such as in the form of a kit) comprising a compound of the invention together with one or more additional therapeutically active agents as a combined preparation for simultaneous, separate or sequential use in the treatment of a disorder for which a Nav1.7 inhibitor is indicated.

It is to be appreciated that all references herein to treatment include curative, palliative and prophylactic treatment.

In the non-limiting Examples and Preparations that are set out later in the description, and in the aforementioned Schemes, the following the abbreviations, definitions and analytical procedures may be referred to:
AcOH is acetic acid;
aq is aqueous;
Boc is tert-butoxycarbonyl;
br is broad;
° C. is degrees celcius;
COMU® is (1-cyano-2-ethoxy-2-oxoethylidenaminooxy)dimethylamino-morpholino-carbenium hexafluorophosphate;
CDCl₃ is deutero-chloroform;
b is chemical shift;
d is doublet;
DCE is dichloroethane;
DCM is dichloromethane; methylene chloride;
DIPEA is N-ethyldiisopropylamine, N,N-diisopropylethylamine;
DMF is N,N-dimethylformamide;
DMSO is dimethyl sulphoxide;
EtOAc is ethyl acetate;
Et₃N is triethylamine;
g is gram;
HBr is hydrobromic acid;
HCl is hydrochloric acid;
H₂O is water;
HPLC is high pressure liquid chromatography;
K₂CO₃ is potassium carbonate;
L is litre;
LCMS is liquid chromatography mass spectrometry (Rt=retention time);
m is multiplet;
M is molar;
mCPBA is metachloroperbenzoic acid;
MeCN is acetonitrile;
MeOH is methanol;
mg is milligram;
MHz is mega Hertz;
min is minutes;
mL is milli litre;
mmol is millimole;
mol is mole;
MS m/z is mass spectrum peak;
NaH is sodium hydride;
NaHCO₃ is sodium hydrogencarbonate;
NaOH is sodium hydroxide;
NH₃ is ammonia;
NHS is N-hydroxysuccinimide;
NMR is nuclear magnetic resonance;
Pd(dppf)Cl₂ is 1,1-bis(diphenylphosphino)ferrocene-palladium(I)dichloride;
PEG is polyethylene glycol;
pH is power of hydrogen;
ppm is parts per million;
psi is pounds per square inch;
q is quartet;
Rt is retention time;
s is singlet;
SCX is strong cation exchange;
t is triplet;
TBME is tert-butyl dimethyl ether;
TEA is triethylamine;
Tf is triflate;
TFA is trifluoroacetic acid;
TFAA is trifluoroacetic acid anhydride;
THF is tetrahydrofuran;
TLC is thin layer chromatography;
μL is microlitre;
μmol is micromol; and
XPhos is 2-dicyclohexylphosphino-2′,4′,6′-triisopropylbiphenyl.

¹H and ¹⁹F Nuclear magnetic resonance (NMR) spectra were in all cases consistent with the proposed structures. Characteristic chemical shifts (6) are given in parts-per-million downfield from tetramethylsilane (for ¹H-NMR) and upfield from trichloro-fluoro-methane (for ¹⁹F NMR) using conventional abbreviations for designation of major peaks: e.g. s, singlet; d, doublet; t, triplet; q, quartet; m, multiplet; br, broad. The following abbreviations have been used for common solvents: CDCl₃, deuterochloroform; d₆-DMSO, deuterodimethylsulphoxide; and CD₃OD, deuteromethanol.

Mass spectra, MS (m/z), were recorded using either electrospray ionisation (ESI) or atmospheric pressure chemical ionisation (APCI). When relevant, and unless stated otherwise, the m/z data provided are for isotopes ¹⁹F, ³⁵Cl and ⁷⁹Br.

Automated Preparative High Performance Liquid Chromatography (Auto-HPLC)

Certain compounds of the Examples and Preparations were purified using Automated Preparative High Performance Liquid Chromatography (HPLC) using a Waters Auto-purification system (2525 Binary Pump, 515 LC Pumps, 2767 Sample manager, and ZQ Mass Spectrometer).

Samples were submitted dissolved in 1 mL of DMSO. Depending on the nature of the compounds and the results of a pre-analysis, the purification was performed under either acidic (‘A-HPLC’), or basic (‘B-HPLC’) conditions at ambient temperature. A-HPLC was carried out on a Sunfire Prep C18 OBD column (19×100 mm, 5 μm). B-HPLC was carried out on an Xterra Prep MS C18 (19×100 mm, 5 μm), both from Waters. A flow rate of 18 mL/min was used with mobile phase A: water +0.1% modifier (v/v) and B: acetonitrile+0.1% modifier (v/v). For acidic runs the modifier was formic acid, for basic run the modifier was diethylamine. A Waters 2525 binary LC pump supplied a mobile phase with a composition of 5% B for 1 min then ran from 5% to 98% B over 6 min followed by a 2 min hold at 98% B.

Detection was achieved using a Waters 2487 dual wavelength absorbance detector set at 225 nm followed in series by a Polymer Labs PL-ELS 2100 detector and a Waters ZQ 2000 4 way MUX mass spectrometer in parallel. The PL-ELS 2100 detector was set at 30° C. with 1.6 L/min supply of Nitrogen. The Waters ZQ MS was tuned with the following parameters:
ES+ Cone voltage: 30 v Capillary: 3.20 kv
ES− Cone voltage: −30 v Capillary: −3.00 kv
Desolvation gas: 600 L/hr
Source Temp: 120° C.
Scan range 150-900 Da

The fraction collection was triggered by both MS and ELSD.

Quality control (QC) analysis was performed using a LCMS method. Acidic runs were carried out on a Sunfire C18 (4.6×50 mm, 5 μm), basic runs were carried out on a Xterra C18 (4.6×50 mm, 5 μm), both from Waters. A flow rate of 1.5 mL/min was used with mobile phase A: water +0.1% modifier (v/v) and B: acetonitrile+0.1% modifier (v/v). For acidic runs the modifier was formic acid, for basic run the modifier was ammonia. A Waters 1525 binary LC pump ran a gradient elution from 5% to 95% B over 3 minutes followed by a 1 minute hold at 95% B. Detection was achieved using a Waters MUX UV 2488 detector set at 225 nm followed in series by a Polymer Labs PL-ELS 2100 detector and a Waters ZQ 2000 4 way MUX mass spectrometer in parallel. The PL-ELS 2100 detector was set at 30° C. with 1.6 L/min supply of Nitrogen. The Waters ZQ MS was tuned with the following parameters:
ES+ Cone voltage: 25 v Capillary: 3.30 kv
ES− Cone voltage: −30 v Capillary: −2.50 kv
Desolvation gas: 800 L/hr
Source Temp: 150° C.
Scan range 160-900 Da

Liquid Chromatography Mass Spectrometry

LCMS conditions were run according to one of the conditions given below (where ratios of solvents are given, the ratios are by volume):

Acidic 2 Minute LCMS
Mobile phase A: 0.1% formic acid in water
Mobile phase B: 0.1% formic acid in 70% methanol: 30% iso-propanol
Column: C18 phase Phenomenex 20×4.0 mm with 3 micron particle size
Gradient: 98-10% A over 1.5 min, 0.3 min hold, 0.2 re-equiilbration, 2 mL/min flow rate
UV: 210 nm-450 nm DAD
Temperature: 75° C.
Or
Mobile phase A: 0.1% formic acid in water
Mobile phase B: 0.1% formic acid in acetonitrile
Column: C18 phase Phenomene×20×4.0 mm with 3 micron particle size
Gradient: 70-2% A over 1.5 min, 0.3 min hold, 0.2 re-equilibration, 1.8 mL/min flow rate
UV: 210 nm-450 nm DAD
Temperature: 75° C.

Acidic 4.5 Minute LCMS
Mobile phase A: 0.05% formic acid in water
Mobile phase B: acetonitrile
Column: Phenomenex Gemini C18 45×45 mm with 5 micron particle size
Gradient: 80-50% A over 0.5 min, 50-2% A over 3 min, 1 min hold, 0.2 min re-equilibration, 2.0 mL/min flow rate
UV: 220 nm-254 nm DAD
Temperature: 40° C.

Acidic 6 Minute LCMS
Mobile phase A: 0.1% formic acid in water
Mobile phase B: 0.1% formic acid in acetonitrile
Column: C18 phase Waters Sunfire 50×4.6 mm with 5 micron particle size
Gradient: 95-5% A over 3 min, 1 min hold, 2 min re-equilibration, 1.5 mL/min flow rate
UV: 210 nm-450 nm DAD
Temperature: 50° C.

Basic 6 Minute LCMS
Mobile phase A: 0.1% ammonium hydroxide in water
Mobile phase B: 0.1% ammonium hydroxide in acetonitrile
Column: C18 phase Fortis 50×4.6 mm with 5 micron particle size
Gradient: 95-5% A over 3 min, 1 min hold, 2 min re-equilibration, 1 mL/min flow rate
UV: 210 nm-450 nm DAD
Temperature: 50° C.

### EXAMPLE 1

### 4-({3-[2-({[2-(1-Acetylpiperidin-4-yl)ethyl]amino}methyl)pyridin-4-yl]-3′-(trifluoromethyl)biphenyl-4-yl}oxy)-3-cyano-N-1,2,4-thiadiazol-5-ylbenzenesulfonamide

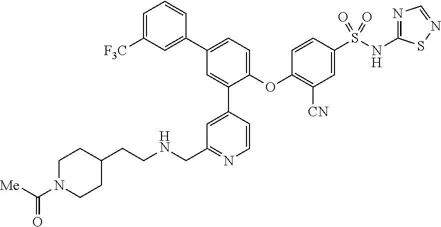 [SMILES: CC(=O)N1CCC(CCNCc2cc(-c3cc(-c4cccc(C(F)(F)F)c4)ccc3Oc3ccc(S(=O)(=O)Nc4ncns4)cc3C#N)ccn2)CC1]

tert-Butyl [2-(1-acetylpiperidin-4-yl)ethyl]({4-[4-{2-cyano-4-[(1,2,4-thiadiazol-5-ylamino)sulfonyl]phenoxy}-3′-(trifluoromethyl) biphenyl-3-yl]pyridin-2-yl}methyl)carbamate (Preparation 4, 49 mg, 0.06 mmol) was dissolved in dioxane (1 mL) and 4M HCl in dioxane (1 mL) was added. The reaction was stirred at room temperature for 4 hours. The solvent was evaporated in vacuo and the residue was azeotroped with methanol. The residue was purified first by reverse phase column chromatography eluting with acetonitrile/water with 0.1% formic acid followed elution through an SCX cartridge to afford the title compound as a white solid (12 mg, 28%).

¹H NMR (400 MHz, CDCl₃): δ ppm 0.92-1.07 (m, 2H), 1.52-1.74 (m, 5H), 1.96 (s, 3H), 2.36 (t, 1H), 2.89 (t, 1H), 3.18 (t, 2H), 3.68 (d, 1H), 4.00-4.43 (m, 3H), 6.64 (d, 1H), 7.33-7.39 (m, 2H), 7.55-7.92 (m, 10H), 8.43 (d, 1H).

¹⁹F NMR (400 MHz, CDCl₃): δ −62.6 (s, 3F).

LCMS Rt=2.37 minutes MS m/z 762 [M+H]⁺

### EXAMPLE 2

### 5-Chloro-2-fluoro-4-{[3-(2-{[(2-piperidin-4-ylethyl)amino]methyl}pyridin-4-yl)-3′-(trifluoromethyl)biphenyl-4-yl]oxy}-N-1,3-thiazol-4-ylbenzenesulfonamide

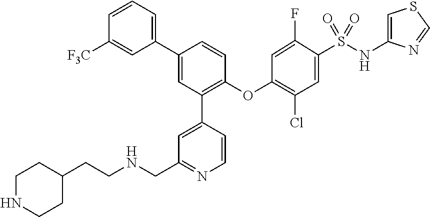 [SMILES: O=S(=O)(Nc1cscn1)c1cc(Cl)c(Oc2ccc(-c3cccc(C(F)(F)F)c3)cc2-c2ccnc(CNCCC3CCNCC3)c2)cc1F]

To a solution of 2,2,2-trichloroethyl ({4-[4-{2-chloro-5-fluoro-4-[(1,3-thiazol-4-ylamino)sulfonyl]phenoxy}-3′-(trifluoromethyl) biphenyl-3-yl]pyridin-2-yl}methyl)(2-piperidin-4-ylethyl)carbamate (Preparation 10, 100 mg, 0.104 mmol) in a diethyl ether/acetic acid (3/1, 7 mL) was added zinc dust (528 mg, 8.34 mmol). The reaction was stirred at room temperature for 1 hour. An aqueous solution of sodium hydrogen carbonate was added until pH=7. The suspension was filtered through a pad of Arbocel and washed with ethyl acetate (20 mL). The aqueous layer was extracted with ethyl acetate (2×10 mL), and the combined organic layers were washed with brine (20 mL), dried over anhydrous magnesium sulfate and concentrated in vacuo. The residue was purified by preparative HPLC to afford the title compound as a colourless solid (21 mg, 26%).

¹H NMR (400 MHz, MeOD-d₄): δ ppm 0.95 (m, 1H), 1.40 (m, 3H), 1.50 (m, 1H), 1.80 (m, 2H), 2.60 (m, 2H), 2.90 (m, 2H), 3.40 (m, 2H), 4.05 (s, 2H), 6.55 (d, 1H), 6.60 (s, 1H), 7.35 (d, 1H), 7.50 (d, 1H), 7.60 (m, 2H), 7.70 (d, 1H), 7.80 (m, 2H), 7.95 (m, 2H), 8.50 (m, 2H), 8.65 (s, 1H).

¹⁹F NMR (400 MHz, MeOD-d₄): δ ppm −64.0 (s, 3F), −119.0 (s, 1F).

LCMS Rt=2.50 minutes MS m/z 746 [M+H]⁺

### EXAMPLE 3

### 6′-{2-cyano-4-[(1,2,4-thiadiazol-5-ylamino)sulfonyl]phenoxy}-N-(2-piperazin-1-ylethyl)-1,1′:3′,1″-terphenyl-3-carboxamide

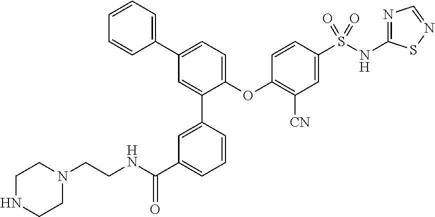 [SMILES: N#Cc1cc(S(=O)(=O)Nc2ncns2)ccc1Oc1ccc(-c2ccccc2)cc1-c1cccc(C(=O)NCCN2CCNCC2)c1]

A solution of hydrochloric acid in dioxane (4M, 4 mL, 100 mmol) was added to tert-butyl 4-(2-{[(6′-{2-cyano-4-[(1,2,4-thiadiazol-5-ylamino)sulfonyl]phenoxy}-1,1′:3′,1″-terphenyl-3-yl)carbonyl]amino}ethyl)piperazine-1-carboxylate (Preparation 20, 100 mg, 0.18 mmol). The resulting reaction mixture was stirred at room temperature for 18 hours. The reaction was concentrated in vacuo to provide the crude product as an orange gum of the hydrochloride salt (193 mg). The crude material was stirred in dioxane (2 mL) and triethylamine (0.4 mL, 1 eq) for 1 hour, then concentrated in vacuo to provide the free parent as an orange gum. The crude material was dissolved in DMSO and purified by reverse phase silica gel column chromatography, eluting with 5-95% MeCN in H₂O+1% NH₃ to afford the title compound as a white solid (28 mg, 16%).

LCMS Rt=1.25 minutes MS m/z 664 [M−H]⁻

### EXAMPLE 4

### 4-[(3″-{[4-(2-aminoethyl)piperazin-1-yl]methyl}-1,1′:3′,1″-terphenyl-4′-yl)oxy]-3-cyano-N-1,2,4-thiadiazol-5-ylbenzenesulfonamide dihydrochloride salt

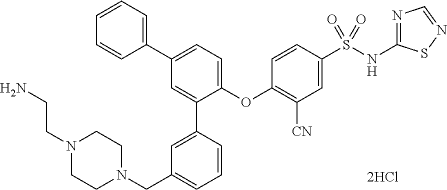 [SMILES: N#Cc1cc(S(=O)(=O)Nc2ncns2)ccc1Oc1ccc(-c2ccccc2)cc1-c1cccc(CN2CCN(CCN)CC2)c1]

To a solution of tert-butyl (2-{4-[(6′-{2-cyano-4-[(1,2,4-thiadiazol-5-ylamino)sulfonyl]phenoxy}-1,1′:3′,1″-terphenyl-3-yl)methyl]piperazin-1-yl}ethyl)carbamate (Preparation 12, 600 mg, 0.8 mmol), dissolved in dichloromethane (10 mL), was added a solution of 4M HCl in 1,4-dioxane (2 mL, 8 mmol) and stirred at room temperature for 18 hours. The solvent was removed in vacuo to afford the title compound as a white solid (570 mg, 98%).

¹H NMR (400 MHz, DMSO-d₆): δ ppm 2.90-3.40 (m, 12H), 4.35 (s, 2H), 6.95 (m, 1H), 7.40-7.65 (m, 7H), 7.80-8.00 (m, 6H), 8.10 (s, 2H), 8.20 (s, 1H), 8.50 (s, 1H).

LCMS Rt=2.20 minutes MS m/z 650 [M−H]⁻

### EXAMPLE 5

### 3-Cyano-4-[(3″-{[(2-piperidin-4-ylethyl)amino]methyl}-1,1′:3′,1″-terphenyl-4′-yl)oxy]-N-1,2,4-thiadiazol-5-ylbenzenesulfonamide sodium salt

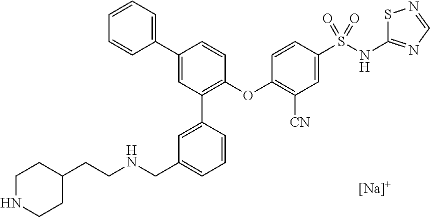

To a solution of N-[(6′-{2-cyano-4-[(1,2,4-thiadiazol-5-ylamino)sulfonyl]phenoxy}-1,1′:3′,1″-terphenyl-3-yl)methyl]-2,2,2-trifluoro-N-(2-piperidin-4-ylethyl)acetamide (Preparation 14, 50 mg, 0.058 mmol) in 3:1 methanol:water (2 mL) was added saturated aqueous sodium carbonate solution (1 mL). The reaction mixture was stirred at reflux for 18 hours, then cooled and partitioned between EtOAc (20 mL) and water (20 mL). The aqueous was further extracted with EtOAc (2×15 mL) then DCM (2×15 mL). The combined organic layers were dried over magnesium sulfate, filtered and concentrated in vacuo to afford the title compound as a white solid (17 mg, 43%).

LCMS Rt=1.88 minutes MS m/z 651 [M+H]⁺

### EXAMPLE 6

### 5-Chloro-2-fluoro-4-{[3-(2-{[(2-piperidin-4-ylethyl)amino]methyl}pyridin-4-yl)-3′-(trifluoromethyl)biphenyl-4-yl]oxy}-N-1,3,4-thiadiazol-2-ylbenzenesulfonamide bis-formate salt

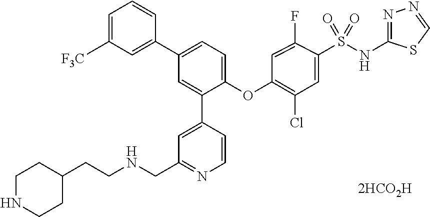 [SMILES: O=S(=O)(Nc1nncs1)c1cc(Cl)c(Oc2ccc(-c3cccc(C(F)(F)F)c3)cc2-c2ccnc(CNCCC3CCNCC3)c2)cc1F ; "2HCO2H"]

To a solution of tert-butyl 4-{2-[({4-[4-{2-chloro-5-fluoro-4-[(1,3,4-thiadiazol-2-ylamino)sulfonyl]phenoxy}-3′-(trifluoromethyl) biphenyl-3-yl]pyridin-2-yl}methyl)amino]ethyl}piperidine-1-carboxylate (Preparation 1, 38 mg, 0.043 mmol) in 1,4-dioxane (1 mL) was added a solution of hydrogen chloride in 1,4-dioxane (4M, 1 mL). The reaction was stirred at room temperature for 2 hours, then concentrated in vacuo. The residue was purified by reverse phase column chromatography eluting with 5-95% acetonitrile in water with 0.1% formic acid to afford the title compound as a colourless solid (21 mg, 60%).

¹H NMR (400 MHz, MeOD-d₄): δ ppm 1.40 (m, 2H), 1.60-1.90 (m, 5H), 2.95 (m, 4H), 3.35 (m, 2H), 4.20 (s, 2H), 6.40 (d, 1H), 7.35 (d, 1H), 7.60 (d, 1H), 7.65 (s, 1H), 7.70 (m, 1H), 7.80 (d, 1H), 7.85 (m, 3H), 7.95 (m, 2H), 8.50 (s, 1H), 8.60 (s, 1H).

¹⁹F NMR (400 MHz, MeOD-d₄): δ ppm −65.0 (s, 3F), −108.0 (s, 1F).

LCMS Rt=2.51 minutes MS m/z 747 [M+H]⁺

### EXAMPLE 7

### 4-({3-[2-(Aminomethyl)pyridin-4-yl]-3′-(trifluoromethyl) biphenyl-4-yl}oxy)-5-chloro-2-fluoro-N-1,3,4-thiadiazol-2-ylbenzenesulfonamide bis-formate salt

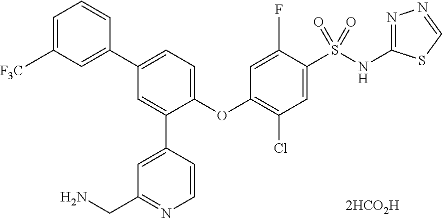 [SMILES: NCc1cc(-c2cc(-c3cccc(C(F)(F)F)c3)ccc2Oc2cc(F)c(S(=O)(=O)Nc3nncs3)cc2Cl)ccn1 ; "2HCO2H"]

To a solution of tert-butyl ({4-[4-(2-chloro-4-{[(2,4-dimethoxybenzyl)(1,3,4-thiadiazol-2-yl)amino]sulfonyl}-5-fluorophenoxy)-3′-(trifluoromethyl) biphenyl-3-yl]pyridin-2-yl}methyl)carbamate (Preparation 3, 1.01 g, 1.14 mmol) in 1,4-dioxane (3 mL) was added a solution of hydrogen chloride in 1,4-dioxane (4M, 3.0 mL, 12 mmol). The reaction was stirred at room temperature for 18 hours and concentrated in vacuo. The resulting residue was purified by reverse phase column chromatography eluting with 5-95% acetonitrile in water with 0.1% formic acid to afford the title compound as a white solid (160 mg, 80%).

¹H NMR (400 MHz, DMSO-d₆): δ ppm 4.20 (s, 2H), 6.90 (d, 1H), 7.20 (d, 1H), 7.70 (m, 5H), 7.90 (d, 1H), 7.95 (s, 1H), 8.05 (m, 2H), 8.40 (br s, 2H), 8.55 (s, 1H), 8.65 (d, 1H).

¹⁹F NMR (400 MHz, DMSO-d₆): δ ppm −62.0 (s, 3F), −107.0 (s, 1F).

LCMS Rt=2.88 minutes MS m/z 636 [M+H]⁺

### EXAMPLE 8

### 2-[2-(2-methoxyethoxy)ethoxy]ethyl [(4-{4-[2-cyano-4-(1,2,4-thiadiazol-5-ylsulfamoyl)phenoxy]-3′-(trifluoromethyl)biphenyl-3-yl}pyridin-2-yl)methyl]carbamate triethylammonium salt

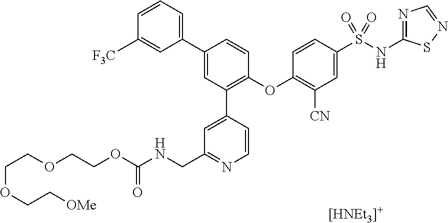 [SMILES: COCCOCCOCCOC(=O)NCc1cc(-c2cc(-c3cccc(C(F)(F)F)c3)ccc2Oc2ccc(S(=O)(=O)Nc3ncns3)cc2C#N)ccn1]

To a solution of 2-(2-(2-methoxyethoxy)ethoxy)ethyl ((4-(4-hydroxy-3′-(trifluoromethyl)-[1,1′-biphenyl]-3-yl)pyridin-2-yl)methyl)carbamate (Preparation 34, 26 mg, 0.049 mmol) in DMSO (0.5 mL) was added potassium carbonate (20 mg, 0.147 mmol) followed by 3-cyano-4-fluoro-N-1,2,4-thiadiazol-5-ylbenzenesulfonamide (WO2010079443, 15 mg, 0.054 mmol). The reaction was heated to 50° C. for 18 hours. The reaction was partitioned between EtOAc and water, the organic layer was collected, dried over sodium sulphate and concentrated in vacuo. The residue was purified using silica gel column chromatography eluting with 10% MeOH in DCM with 2% TEA to afford the title compound.

¹H NMR (500 MHz, MeOH-d₄): δ ppm 3.44-3.51 (m, 2H), 3.53-3.61 (m, 9H), 3.63-3.72 (m, 2H), 4.13-4.21 (m, 2H), 4.42 (s, 2H), 6.89-7.01 (m, 1H), 7.36-7.45 (m, 1H), 7.48-7.58 (m, 1H), 7.58-7.65 (m, 1H), 7.66-7.75 (m, 2H), 7.83-7.92 (m, 2H), 7.92-8.04 (m, 4H), 8.07-8.18 (m, 1H), 8.41-8.53 (m, 1H).

MS m/z 799 [M+H]⁺

### EXAMPLE 9

### 3-cyano-4-((3-(2-(3-oxo-7,10,13,16-tetraoxa-2,4-diazaheptadecyl)pyridin-4-yl)-3′-(trifluoromethyl)-[1,1′-biphenyl]-4-yl)oxy)-N-(1,2,4-thiadiazol-5-yl)benzenesulfonamide triethylammonium salt

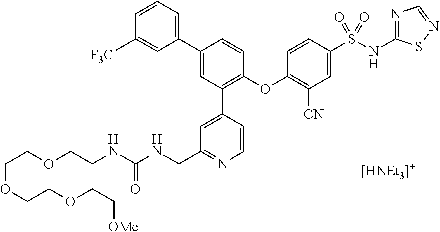 [SMILES: COCCOCCOCCOCCNC(=O)NCc1cc(-c2cc(-c3cccc(C(F)(F)F)c3)ccc2Oc2ccc(S(=O)(=O)Nc3ncns3)cc2C#N)ccn1]

The title compound was prepared according to the method described for Example 8 using 3-cyano-4-fluoro-N-1,2,4-thiadiazol-5-ylbenzenesulfonamide (WO2010079443) and 1-((4-(4-hydroxy-3′-(trifluoromethyl)-[1,1′-biphenyl]-3-yl)pyridin-2-yl)methyl)-3-(2,5,8,11-tetraoxatridecan-13-yl)urea (Preparation 33) at 90° C. for 18 hours.

¹H NMR (500 MHz, MeOH-d₄): δ ppm 3.20-3.60 (m, 19H), 4.40 (s, 2H), 6.95 (m, 1H), 7.20 (m, 1H), 7.30 (m, 1H), 7.40 (m, 1H), 7.70-7.75 (m, 2H), 7.90 (m, 2H), 7.95-8.05 (m, 4H), 8.10 (m, 1H), 8.45 (m, 1H). LCMS Rt=1.59 minutes MS m/z 842 [M+H]⁺

### EXAMPLE 10

### 4-((3-(2-(2,8,11,14,17-pentaoxa-5-azaoctadecyl)pyridin-4-yl)-3′-(trifluoromethyl)-[1,1′-biphenyl]-4-yl)oxy)-3-cyano-N-(1,2,4-thiadiazol-5-yl)benzenesulfonamide

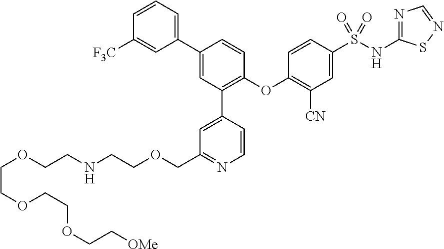 [SMILES: COCCOCCOCCOCCNCCOCc1cc(-c2cc(-c3cccc(C(F)(F)F)c3)ccc2Oc2ccc(S(=O)(=O)Nc3ncns3)cc2C#N)ccn1]

The title compound was prepared according to the method described for Example 8 using 3-cyano-4-fluoro-N-1,2,4-thiadiazol-5-ylbenzenesulfonamide (WO2010079443) and 3-(2-(2,8,11,14,17-pentaoxa-5-azaoctadecyl)pyridin-4-yl)-3′-(trifluoromethyl)-[1,1′-biphenyl]-4-ol (Preparation 32) using potassium phosphate as base at 65° C. for 18 hours. The residue was purified using Preparative HPLC.

LCMS Rt=2.54 minutes MS m/z 843 [M+H]⁺

### EXAMPLE 11

### (4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methyl (2,5,8,11-tetraoxatridecan-13-yl)carbamate

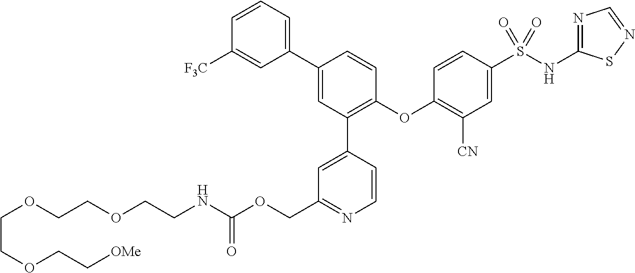 [SMILES: COCCOCCOCCOCCNC(=O)OCc1cc(-c2cc(-c3cccc(C(F)(F)F)c3)ccc2Oc2ccc(S(=O)(=O)Nc3ncns3)cc2C#N)ccn1]

The title compound was prepared according to the method described for Example 8 using 3-cyano-4-fluoro-N-1,2,4-thiadiazol-5-ylbenzenesulfonamide (WO2010079443) and (4-(4-hydroxy-3′-(trifluoromethyl)-[1,1′-biphenyl]-3-yl)pyridin-2-yl)methyl (2,5,8,11-tetraoxatridecan-13-yl)carbamate (Preparation 27) using potassium phosphate as base at 65° C. for 18 hours followed by the addition of potassium carbonate and further heating at 90° C. for 18 hours. The residue was purified using Preparative HPLC.

LCMS Rt=2.42 minutes MS m/z 843 [M+H]⁺

### EXAMPLE 12

### 3-cyano-4-({3-[2-(2,5,8,11,14,17,20,23,26,29,32,35-dodecaoxa-38-azanonatriacontan-39-yl)pyridin-4-yl]-3′-(trifluoromethyl)biphenyl-4-yl}oxy)-N-(1,2,4-thiadiazol-5-yl)benzenesulfonamide

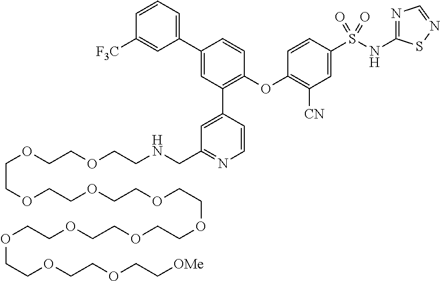 [SMILES: COCCOCCOCCOCCOCCOCCOCCOCCOCCOCCOCCOCCNCc1cc(-c2cc(-c3cccc(C(F)(F)F)c3)ccc2Oc2ccc(S(=O)(=O)Nc3ncns3)cc2C#N)ccn1]

To a solution of tert-butyl (2,5,8,11,14,17,20,23,26,29,32,35-dodecaoxaheptatriacontan-37-yl) ((4-(4-hydroxy-3′-(trifluoromethyl)-[1,1′-biphenyl]-3-yl)pyridin-2-yl)methyl)carbamate (Preparation 28, 108 mg, 0.109 mmol) in DMSO (2 mL) was added potassium phosphate (70 mg, 0.327 mmol) and 3-cyano-N-(2,4-dimethoxybenzyl)-4-fluoro-N-(1,2,4-thiadiazol-5-yl)benzenesulfonamide (WO2010079443, 48 mg, 0.11 mmol). The reaction was stirred at room temperature for 18 hours. To the reaction was added water and EtOAc. The organic layer was separated, the aqueous layer was further extracted with EtOAc, the organic layers were combined, washed with brine, dried over sodium sulfate and concentrated in vacuo. The residue was purified using silica gel column chromatography eluting with 0-10% MeOH in DCM and the residue was dissolved in DCM (2.5 mL) and treated with TFA (200 uL), with stirring for 2 hours. 5N HCl (100 uL) was then added and the reaction stirred for 1 hour. The reaction was concentrated in vacuo azeotroping with DCM, EtOAc and heptanes. The residue was dissolved in 10% MeOH in DCM (10 mL) and basified with MP-carbonate before concentrating in vacuo. The residue was purified using silica gel column chromatography eluting with 0-10% MeOH in DCM to afford the title compound (35 mg, 35%).

¹H NMR (400 MHz, CDCl₃): δ ppm 3.34-3.40 (s, 3H), 3.51-3.55 (m, 2H), 3.56-3.66 (m, 42H), 3.67-3.71 (m, 2H), 3.88 (m, 2H), 4.47 (s, 2H), 6.66 (d, 1H), 7.37 (d, 1H), 7.48 (d, 2H) 7.61 (s, 1H), 7.65-7.68 (m, 2H), 7.72-7.78 (m, 1H), 7.79-7.84 (m, 1H), 7.85 (s, 1H), 7.88 (s, 1H), 7.96 (s, 1H), 8.00 (d, 1H), 8.50 (d, 1H).

LCMS Rt=1.65 minutes MS m/z 1149 [M−H]⁻

### EXAMPLE 13

### 3-cyano-4-({3-[2-(2,5,8,11,14-pentaoxapentadec-1-yl)pyridin-4-yl]-3′-(trifluoromethyl)biphenyl-4-yl}oxy)-N-(1,2,4-thiadiazol-5-yl)benzenesulfonamide

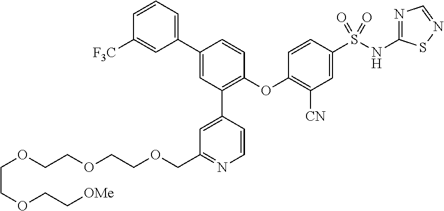 [SMILES: COCCOCCOCCOCCOCc1cc(-c2cc(-c3cccc(C(F)(F)F)c3)ccc2Oc2ccc(S(=O)(=O)Nc3ncns3)cc2C#N)ccn1]

The title compound was prepared according to the method described by Example 12 using 3-cyano-N-(2,4-dimethoxybenzyl)-4-fluoro-N-(1,2,4-thiadiazol-5-yl)benzenesulfonamide (WO2010079443) and 3-(2-(2,5,8,11,14-pentaoxapentadecyl)pyridin-4-yl)-3′-(trifluoromethyl)-[1,1′-biphenyl]-4-ol (Preparation 30).

¹H NMR (500 MHz, MeOH-d₄): δ ppm 3.20-3.30 (m, 11H), 3.45 (m, 2H), 3.50-3.65 (m, 6H), 4.70 (s, 2H), 6.95 (m, 1H), 7.50 (m, 1H), 7.60 (m, 1H), 7.70 (m, 2H), 7.80 (m, 1H), 7.90-8.00 (m, 3H), 8.02 (m, 2H), 8.10-8.20 (m, 2H), 8.55 (m, 1H).

MS m/z 800 [M+H]⁺

### EXAMPLE 14

### 2-((4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methoxy)-N-(2,5,8,11-tetraoxatridecan-13-yl)acetamide

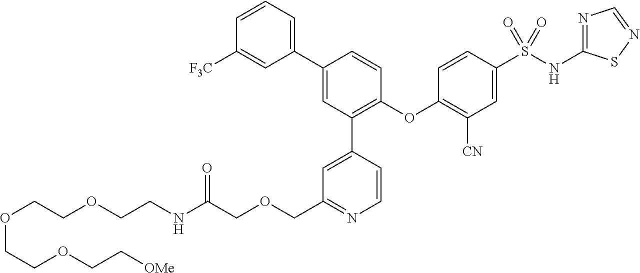 [SMILES: COCCOCCOCCOCCNC(=O)COCc1cc(-c2cc(-c3cccc(C(F)(F)F)c3)ccc2Oc2ccc(S(=O)(=O)Nc3ncns3)cc2C#N)ccn1]

The title compound was prepared according to the method described by Example 12 using 3-cyano-N-(2,4-dimethoxybenzyl)-4-fluoro-N-(1,2,4-thiadiazol-5-yl)benzenesulfonamide (WO2010079443) and 2-((4-(4-hydroxy-3′-(trifluoromethyl)-[1,1′-biphenyl]-3-yl)pyridin-2-yl)methoxy)-N-(2,5,8,11-tetraoxatridecan-13-yl)acetamide (Preparation 31).

¹H NMR (500 MHz, CDCl₃): δ ppm 3.44 (s, 3H), 3.46-3.52 (m, 2H), 3.59-3.75 (m, 16H), 4.62 (s, 2H), 6.59 (d, 1H), 7.33 (dd, 1H), 7.43 (d, 2H), 7.63 (d, 1H), 7.66-7.71 (m, 2H), 7.75-7.84 (m, 2H), 7.87 (s, 1H), 7.94 (dd, 1H), 7.99 (s, 1H), 8.10 (d, 1H), 8.55 (d, 1H).

MS m/z 857 [M+H]⁺

### EXAMPLE 15

### 4-((3-(2-(5,8,11,14-tetraoxa-2-azapentadecyl)pyridin-4-yl)-3′-(trifluoromethyl)-[1,1′-biphenyl]-4-yl)oxy)-3-cyano-N-(1,2,4-thiadiazol-5-yl)benzenesulfonamide

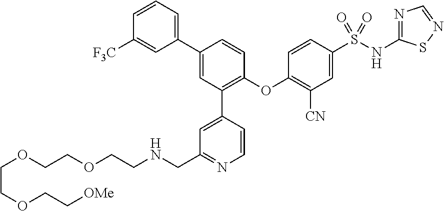 [SMILES: COCCOCCOCCOCCNCc1cc(-c2cc(-c3cccc(C(F)(F)F)c3)ccc2Oc2ccc(S(=O)(=O)Nc3ncns3)cc2C#N)ccn1]

To a solution of tert-butyl ((4-(4-hydroxy-3′-(trifluoromethyl)-[1,1′-biphenyl]-3-yl)pyridin-2-yl)methyl)(2,5,8,11-tetraoxatridecan-13-yl)carbamate (Preparation 29, 48 mg, 0.10 mmol) in DMSO (2 mL) was added potassium phosphate (58 mg, 0.273 mmol) and 3-cyano-N-(2,4-dimethoxybenzyl)-4-fluoro-N-(1,2,4-thiadiazol-5-yl)benzenesulfonamide (WO2010079443, 28 mg, 0.10 mmol). The reaction was stirred at room temperature for 18 hours followed by 90° C. for 2 hours. To the reaction was added water and EtOAc.

The organic layer was separated, the aqueous layer was further extracted with EtOAc, the organic layers were combined, washed with brine, dried over sodium sulfate and concentrated in vacuo. The residue was purified using 0-10% MeOH in DCM and dissolved in DCM (2.5 mL) and treated with TFA (200 uL), with stirring for 2 hours. 5N HCl in isopropenol (200 uL) was then added and the reaction stirred for 1 hour. The reaction was concentrated in vacuo and purified using preparative HPLC to afford the title compound (30 mg, 95%).

LCMS Rt=2.99 minutes MS m/z 799 [M+H]⁺

### EXAMPLE 16

### 4-{[3″-({[2-(1-Acetylpiperidin-4-yl)ethyl]amino}methyl)-1,1′:3′,1″-terphenyl-4′-yl]oxy}-3-cyano-N-1,2,4-thiadiazol-5-ylbenzenesulfonamide

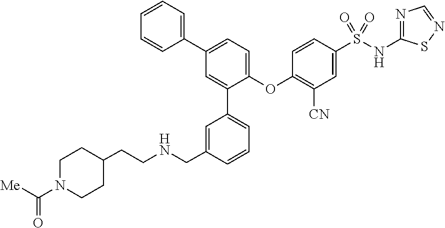 [SMILES: CC(=O)N1CCC(CCNCc2cccc(-c3cc(-c4ccccc4)ccc3Oc3ccc(S(=O)(=O)Nc4ncns4)cc3C#N)c2)CC1]

To a solution of 4-{[3″-({[2-(1-acetylpiperidin-4-yl)ethyl]amino}methyl)-1,1′:3′,1″-terphenyl-4′-yl]oxy}-3-cyano-N-(2,4-dimethoxybenzyl)-N-1,2,4-thiadiazol-5-ylbenzenesulfonamide (Preparation 23, 1.22 g, 1.45 mmol) in dichloromethane (10 mL) was added trifluoroacetic acid (540 μL, 7.25 mmol). The reaction mixture was stirred for 18 hours at room temperature. Methanol (50 mL) was added then the reaction mixture was concentrated in vacuo. The crude material was purified by preparative reverse phase HPLC, then by silica gel column chromatography eluting 10-50% methanol in ethyl acetate to afford the title compound as a white solid (346 mg, 34%).

¹H NMR (400 MHz, CDCl₃): δ ppm 1.13 (m, 2H), 1.63 (m, 3H), 1.75 (m, 2H), 2.08 (s, 3H), 2.60 (m, 1H), 3.01 (m, 2H), 3.09 (m, 1H), 3.89 (m, 1H), 4.12 (s, 2H), 4.48 (m, 1H), 6.72 (m, 1H), 7.27 (m, 1H), 7.33 (m, 1H), 7.41 (m, 2H), 7.47 (m, 2H), 7.53 (m, 1H), 7.60 (m, 1H), 7.68 (m, 2H), 7.73 (m, 1H), 7.79 (m, 2H), 7.96 (m, 2H).

LCMS Rt=2.32 minutes MS m/z 693 [M+H]⁺

### EXAMPLE 17

### 4-((3-(2-(((2-(1-(2,5,8,11,14,17,20,23,26,29,32,35-dodecaoxaoctatriacontan-38-oyl)piperidin-4-yl)ethyl)amino)methyl)pyridin-4-yl)-3′-(trifluoromethyl)-[1,1′-biphenyl]-4-yl)oxy)-3-cyano-N-(1,2,4-thiadiazol-5-yl)benzenesulfonamide

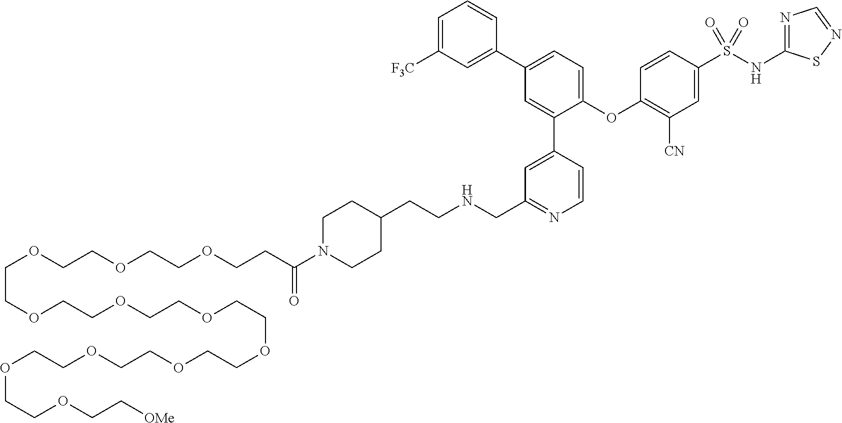 [SMILES: COCCOCCOCCOCCOCCOCCOCCOCCOCCOCCOCCOCCC(=O)N1CCC(CCNCc2cc(-c3cc(-c4cccc(C(F)(F)F)c4)ccc3Oc3ccc(S(=O)(=O)Nc4ncns4)cc3C#N)ccn2)CC1]

To a solution of 2,5-dioxopyrrolidin-1-yl 2,5,8,11,14,17,20,23,26,29,32,35-dodecaoxaoctatriacontan-38-oate (m-dPEG12-NHS ester, 29 mg, 0.042 mmol) in DCM (1 mL) was added TEA (18 uL, 0.124 mmol) followed by tert-butyl {[4-(3′-tert-butyl-4-{2-cyano-4-[(1,2,4-thiadiazol-5-ylamino)sulfonyl]phenoxy}biphenyl-3-yl)pyridin-2-yl]methyl}(2-piperidin-4-ylethyl)carbamate (Preparation 5, 34 mg, 0.042 mmol) and the reaction was stirred at room temperature for 2 hours. The reaction was concentrated in vacuo and dissolved in dioxane (1 mL). The solution was treated with 4M HCl in dioxane (1 mL) and stirred at room temperature for 1 hour. The reaction was concentrated in vacuo and purified using preparative HPLC to afford the title compound (23 mg, 43%).

LCMS Rt=2.62 minutes MS m/z 1290[M+H]⁺

### EXAMPLE 18a

### 3-Cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide bis-formate salt

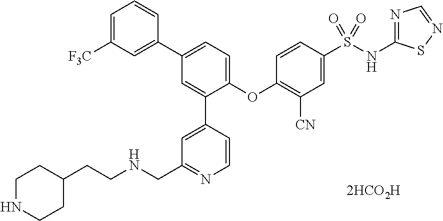 [SMILES: N#Cc1cc(S(=O)(=O)Nc2ncns2)ccc1Oc1ccc(-c2cccc(C(F)(F)F)c2)cc1-c1ccnc(CNCCC2CCNCC2)c1]

To a solution of tert-butyl 4-{2-[({4-[4-{2-cyano-4-[(1,2,4-thiadiazol-5-ylamino)sulfonyl]phenoxy}-3′-(trifluoromethyl) biphenyl-3-yl]pyridin-2-yl}methyl)amino]ethyl}piperidine-1-carboxylate (Preparation 19, 35 mg, 0.04 mmol) in 1,4-dioxane (1 mL) was added a solution of hydrogen chloride in 1,4-dioxane (4M, 1 mL). The reaction was stirred at room temperature for 2 hours, then concentrated in vacuo. The residue was purified by reverse phase column chromatography eluting with 5-95% acetonitrile in water with 0.1% formic acid to afford the title compound as a colourless solid (20 mg, 65%).

¹H NMR (400 MHz, MeOD-d₄): δ ppm 1.40 (m, 1H), 1.75 (m, 2H), 1.95 (d, 2H), 2.95 (t, 2H), 3.10 (m, 2H), 3.40 (d, 2H), 3.40 (d, 2H), 4.40 (s, 2H), 6.75 (d, 1H), 7.45 (d, 1H), 7.55 (d, 1H), 7.60-7.70 (m, 3H), 7.80-8.00 (m, 5H), 8.20 (br s, 2H), 8.50 (s, 1H).

¹⁹F NMR (400 MHz, MeOD-d₄): δ ppm −64.5 (s, CF₃).

LCMS Rt=2.44 minutes MS m/z 720 [M+H]⁺

### EXAMPLE 18b

### 3-Cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide trihydrochloride salt

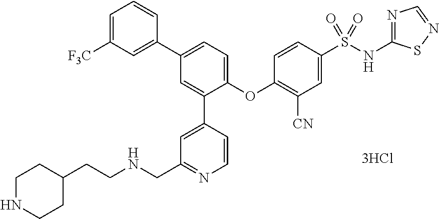 [SMILES: N#Cc1cc(S(=O)(=O)Nc2ncns2)ccc1Oc1ccc(-c2cccc(C(F)(F)F)c2)cc1-c1ccnc(CNCCC2CCNCC2)c1]

To a solution of tert-butyl 4-{2-[({4-[4-{2-cyano-4-[(1,2,4-thiadiazol-5-ylamino)sulfonyl]phenoxy}-3′-(trifluoromethyl) biphenyl-3-yl]pyridin-2-yl}methyl)amino]ethyl}piperidine-1-carboxylate (Preparation 19, 26 g, 32 mmol) in methanol (300 mL) was added 5M HCl in iso-propyl alcohol (130 mL, 634 mmol). The mixture was stirred at room temperature overnight, the mixture filtered and the solid washed with 50% methanol in iso-propyl alcohol (3×65 mL) and dried under vacuum to afford afford the crude title compound as a colourless solid (28.7 g, 109%).

A suspension of crude 3-cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide trihydrochloride salt (56.1 g) in methanol (700 mL) was stirred for 30 minutes then treated with iso-propyl alcohol (500 mL), heated to 45° C. and stirred overnight. The slurry was filtered, washed with 60% methanol in iso-propyl alcohol (3×65 mL) to afford the title compound as a white solid (29.2 g). The filtrate was partially concentrated under reduced pressure to a volume of approximately 250 mL and the resulting slurry was stirred at room temperature for 2 hours. The mixture was filtered and the solid washed with iso-propyl alcohol (2×50 mL) and dried under vacuum to afford further title compound as an off-white solid (13.7 g).

1H NMR (600 MHz, MeOD-d₄) b ppm 1.47 (m, 2H), 1.79 (br. s., 3H), 1.98 (br. d, 2H) 3.01 (t, 2H), 3.19 (br. s., 2H), 3.40 (d, 2H), 4.51 (br. s., 2H), 7.03 (d, 1H), 7.50 (d, 1H), 7.67-7.77 (m, 2H), 7.81 (d, 1H), 7.96 (t, 2H), 7.98-8.09 (m, 4H), 8.15 (s, 1H), 8.23 (s, 1H), 8.71 (d, 1H).

CHN calculated for C₃₈H₃₂Cl₃F₃N₇O₃S₂ C, 50.70; H, 4.25; N, 11.82; Cl, 12.83. CHN Found C, 49.99; H, 4.36; N, 11.54; Cl, 12.30.

### EXAMPLE 18c

### 3-Cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide

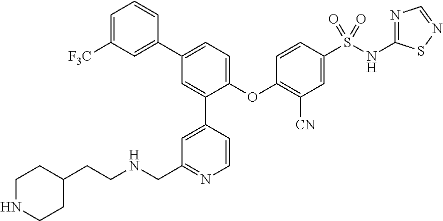 [SMILES: N#Cc1cc(S(=O)(=O)Nc2ncns2)ccc1Oc1ccc(-c2cccc(C(F)(F)F)c2)cc1-c1ccnc(CNCCC2CCNCC2)c1]

To a mixture of 3-cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide trihydrochloride salt (Example 18b, 150 mg, 0.18 mmol) in ethyl acetate (2.2 mL) and water (1.5 mL) was added 1N aqueous sodium hydroxide solution (0.45 mL, 0.18 mmol) to pH 6-7. The mixture was stirred at room temperature for 1 hour and the resulting slurry was filtered. The solid was washed with water and dried in a vacuum oven at 50° C. for 16 hours to afford the title compound as white solid (100 mg, 77%).

1H NMR (400 MHz, DMSO-d6) δ ppm 1.08-1.25 (m, 2H), 1.30 (q, 2H), 1.44-1.62 (m, 1H), 1.68 (d, 2H), 2.42 (t, 2H), 2.80 (td, 2H), 3.21 (d, 2H), 3.80 (s, 2H), 6.94 (d, 1H), 7.43-7.55 (m, 2H), 7.61 (s, 1H), 7.68-7.83 (m, 2H), 7.84-7.93 (m, 2H), 7.96 (dd, 1H), 8.01 (dd, 2H), 8.09-8.20 (m, 2H), 8.52 (d, 1H)

### EXAMPLE 19

### N-((4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methyl)-2,5,8,11,14,17,20,23,26,29,32,35-dodecaoxaoctatriacontan-38-amide

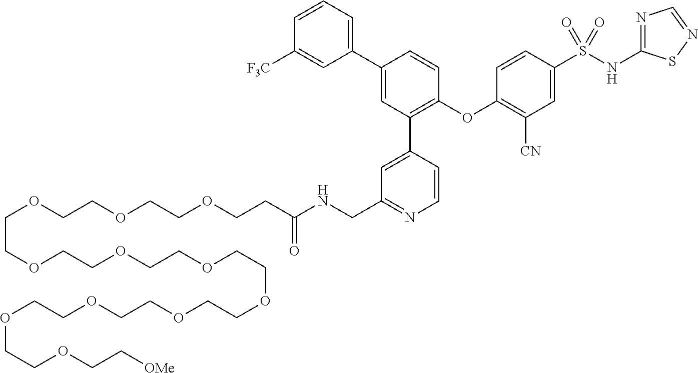 [SMILES: COCCOCCOCCOCCOCCOCCOCCOCCOCCOCCOCCOCCC(=O)NCc1cc(-c2cc(-c3cccc(C(F)(F)F)c3)ccc2Oc2ccc(S(=O)(=O)Nc3ncns3)cc2C#N)ccn1]

To a solution of 4-({3-[2-(Aminomethyl)pyridin-4-yl]-3′-(trifluoromethyl)biphenyl-4-yl}oxy)-3-cyano-N-1,2,4-thiadiazol-5-ylbenzenesulfonamide bis-formate salt (Example 26, 50 mg, 0.082 mmol) in DMF (2 mL) was added 2,5-dioxopyrrolidin-1-yl 2,5,8,11,14,17,20,23,26,29,32,35-dodecaoxaoctatriacontan-38-oate (m-dPEG12-NHS ester, 51 mg, 0.082 mmol) followed by TEA (0.1 mL, 0.41 mmol) and the reaction was stirred at room temperature for 18 hours. The reaction was concentrated in vacuo and the residue purified using reverse phase column chromatography eluting with acetonitrile and water to afford the title compound (53 mg, 100%).

¹H NMR (400 MHz, CDCl₃): δ ppm 2.45 (m, 2H), 3.45-3.55 (m, 47H), 3.75 (m, 2H), 4.42 (m, 2H), 6.70 (m, 1H), 7.25 (m, 1H), 7.35 (m, 2H), 7.50-7.85 (m, 7H), 8.00-8.10 (m, 2H), 8.45 (m, 1H).

LCMS Rt=2.47 minutes MS m/z 1177 [M−H]⁻

### EXAMPLE 20

### 4-((3-(2-((3-((2,5,8,11-tetraoxatridecan-13-yl)oxy)azetidin-1-yl)methyl)pyridin-4-yl)-3′-(trifluoromethyl)-[1,1′-biphenyl]-4-yl)oxy)-3-cyano-N-(2,4-dimethoxybenzyl)-N-(1,2,4-thiadiazol-5-yl)benzenesulfonamide hemi trifluoroacetate salt

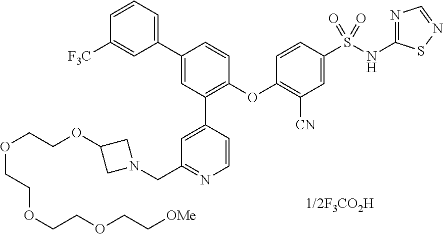 [SMILES: COCCOCCOCCOCCOC1CN(Cc2cc(-c3cc(-c4cccc(C(F)(F)F)c4)ccc3Oc3ccc(S(=O)(=O)Nc4ncns4)cc3C#N)ccn2)C1]

To a solution of 3-cyano-N-(2,4-dimethoxybenzyl)-4-((3-(2-(hydroxymethyl)pyridin-4-yl)-3′-(trifluoromethyl)-[1,1′-biphenyl]-4-yl)oxy)-N-(1,2,4-thiadiazol-5-yl)benzenesulfonamide (Preparation 18, 50 mg, 0.066 mmol) in DCM (1 mL) was added DIPEA (0.027 mL, 0.165 mmol) followed by mesyl chloride (7.6 mg, 0.066 mmol) and the reaction was stirred at room temperature for 2 hours. The solution was purified directly using silica gel column chromatography eluting with 0-100% EtOAc in heptanes. The residue was dissolved in DCM (1 mL) and DIPEA (0.027 mL, 0.165 mmol) was added followed by 3-((2,5,8,11-tetraoxatridecan-13-yl)oxy)azetidine (Preparation 83, 24 mg, 0.091 mmol) and the reaction was stirred at room temperature for 18 hours. The reaction was purified directly using silica gel column chromatography eluting with 0-10% MeOH in DCM. The residue was dissolved in DCM (2 mL) and treated with TFA (0.25 mL) and stirred at room temperature for 4 hours. The reaction was concentrated in vacuo and purified using silica gel column chromatography eluting with 0-10% MeOH in DCM to afford the title compound (19 mg, 31%).

¹H NMR (400 MHz, DMSO-d₆): δ ppm 3.20 (s, 3H), 3.30-3.60 (m, 16H), 4.00-4.10 (m, 2H), 4.30-4.40 (m, 3H), 4.60 (m, 2H), 7.00 (d, 1H), 7.53 (d, 1H), 7.63-7.81 (m, 3H), 7.92 (m, 1H), 7.94 (m, 1H), 7.98-8.10 (m, 2H), 8.10-8.15 (m, 4H), 8.64 (d, 1H).

¹⁹F NMR (300 MHz): δ ppm −61.3 (s, 3F), −73.9 (s, 1.6F).

MS m/z 855 [M+H]⁺

### EXAMPLE 21

### (R)-4-((3-(2-((3-((2,5,8,11-tetraoxatridecan-13-yl)oxy)pyrrolidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide trifluoroacetate salt

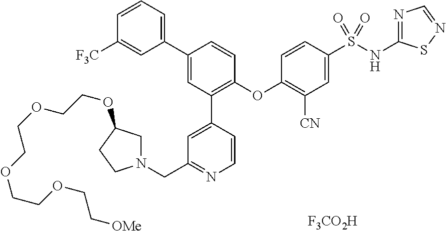

The title compound was prepared according to the method described for Example 20 using 3-cyano-N-(2,4-dimethoxybenzyl)-4-((3-(2-(hydroxymethyl)pyridin-4-yl)-3′-(trifluoromethyl)-[1,1′-biphenyl]-4-yl)oxy)-N-(1,2,4-thiadiazol-5-yl)benzenesulfonamide (Preparation 18) and (R)-3-((2,5,8,11-tetraoxatridecan-13-yl)oxy)pyrrolidine (Preparation 64).

¹H NMR (400 MHz, MeOD-d₄): δ ppm 2.10 (br m, 2H), 3.20 (s, 3H), 3.35-3.52 (m, 20H), 4.27 (br s, 1H), 4.58 (br s, 2H), 7.01 (d, 1H), 7.56 (d, 1H), 7.68 (dd, 1H), 7.73-7.81 (m, 3H), 7.92-7.95 (m, 1H), 7.99-8.02 (m, 2H), 8.10-8.13 (m, 3H), 8.18 (m, 1H), 8.69 (m, 1H). ¹⁹F NMR (300 MHz): δ ppm −61.16 (s, 3F), −73.99 (s, 3F).

MS m/z 869 [M+H]⁺

### EXAMPLE 22

### (S)-4-((3-(2-((3-((2,5,8,11-tetraoxatridecan-13-yl)oxy)pyrrolidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide hemi-trifluoroacetate salt

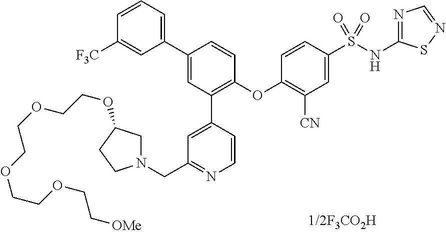 [SMILES: COCCOCCOCCOCCO[C@H]1CCN(Cc2cc(-c3cc(-c4cccc(C(F)(F)F)c4)ccc3Oc3ccc(S(=O)(=O)Nc4ncns4)cc3C#N)ccn2)C1]

The title compound was prepared according to the method described for Example 20 using 3-cyano-N-(2,4-dimethoxybenzyl)-4-((3-(2-(hydroxymethyl)pyridin-4-yl)-3′-(trifluoromethyl)-[1,1′-biphenyl]-4-yl)oxy)-N-(1,2,4-thiadiazol-5-yl)benzenesulfonamide (Preparation 18) and (S)-3-((2,5,8,11-tetraoxatridecan-13-yl)oxy)pyrrolidine (Preparation 65).

¹H NMR (300 MHz, MeOD-d₄): δ ppm 2.09 (br m, 2H), 3.20 (s, 3H), 3.37-3.52 (m, 20H), 4.27 (br m, 1H), 4.58 (m, 2H), 6.99 (d, 1H), 7.53-7.56 (d, 1H), 7.66 (m, 1H), 7.42-7.78 (m, 3H), 7.90-7.94 (m, 1H), 7.99-8.02 (m, 2H), 8.10-8.12 (m, 4H), 8.67 (m, 1H).

¹⁹F NMR (300 MHz): δ ppm −61.26 (s, 3F), −73.92 (s, 1.6F).

MS m/z 869 [M+H]⁺

### EXAMPLE 23

### 4-((3-(2-((4-((2,5,8,11-tetraoxatridecan-13-yl)oxy)piperidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide trifluoroacetate salt

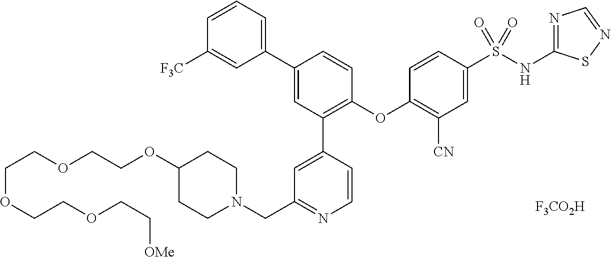 [SMILES: COCCOCCOCCOCCOC1CCN(Cc2cc(-c3cc(-c4cccc(C(F)(F)F)c4)ccc3Oc3ccc(S(=O)(=O)Nc4ncns4)cc3C#N)ccn2)CC1 ; "F3CO2H"]

The title compound was prepared according to the method described for Example 20 using 3-cyano-N-(2,4-dimethoxybenzyl)-4-((3-(2-(hydroxymethyl)pyridin-4-yl)-3′-(trifluoromethyl)-[1,1′-biphenyl]-4-yl)oxy)-N-(1,2,4-thiadiazol-5-yl)benzenesulfonamide (Preparation 18) and 4-((2,5,8,11-tetraoxatridecan-13-yl)oxy)piperidine hydrochloride (Preparation 66). The final residue was purified using Preparative HPLC.

¹H NMR (400 MHz, DMSO-d₆): δ ppm 1.70-1.95 (br m, 2H), 1.95-2.10 (br m, 2H), 3.10-3.70 (m, 24H), 4.50 (m, 2H), 7.10 (d, 1H), 7.60 (d, 1H), 7.70-7.74 (m, 1H), 7.80-7.83 (m, 3H), 7.98-8.10 (m, 3H), 8.12-8.20 (m, 2H), 8.22-8.26 (m, 1H), 8.43 (br s, 1H), 8.74-8.78 (m, 1H).

MS m/z 883 [M+H]⁺

### EXAMPLE 24

### 4-((3-(2-(4-(2,5,8,11,14,17,20,23,26,29,32,35-dodecaoxaoctatriacontan-38-oyl)piperazin-1-yl)pyridin-4-yl)-4′-(trifluoromethyl)-[1,1′-biphenyl]-4-yl)oxy)-3-cyano-N-(1,2,4-thiadiazol-5-yl)benzenesulfonamide

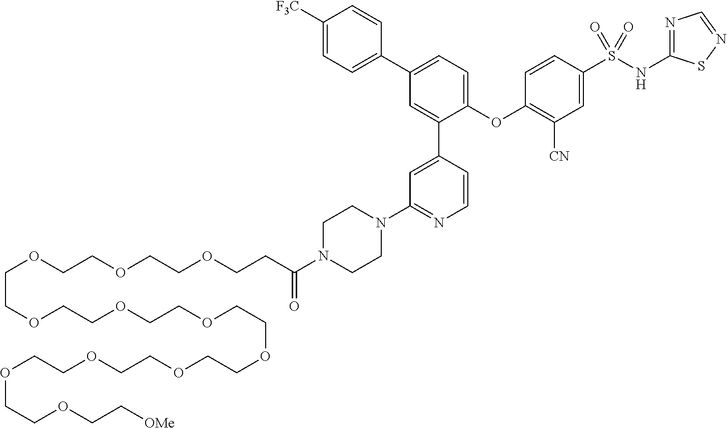 [SMILES: COCCOCCOCCOCCOCCOCCOCCOCCOCCOCCOCCOCCC(=O)N1CCN(c2cc(-c3cc(-c4ccc(C(F)(F)F)cc4)ccc3Oc3ccc(S(=O)(=O)Nc4ncns4)cc3C#N)ccn2)CC1]

To a solution of 3-cyano-4-((3-(2-(piperazin-1-yl)pyridin-4-yl)-4′-(trifluoromethyl)-[1,1′-biphenyl]-4-yl)oxy)-N-(1,2,4-thiadiazol-5-yl)benzenesulfonamide (Preparation 2, 50 mg, 0.07 mmol) in DMF (2 mL) was added triethylamine (0.03 mL, 0.35 mmol) followed by 2,5-dioxopyrrolidin-1-yl 2,5,8,11,14,17,20,23,26,29,32,35-dodecaoxaoctatriacontan-38-oate (m-dPEG12-NHS ester, 50 mg, 0.07 mmol) and the reaction was stirred at room temperature for 18 hours. The reaction was concentrated in vacuo and purified using preparative HPLC to afford the title compound.

LCMS Rt=2.49 minutes MS m/z 1232 [M−H]⁻

### EXAMPLE 25

### 3-Cyano-N-1,2,4-thiadiazol-5-yl-4-{[3-{2-[({2-[1-(trifluoroacetyl)piperidin-4-yl]ethyl}amino)methyl]pyridin-4-yl}-3′-(trifluoromethyl)biphenyl-4-yl]oxy}benzenesulfonamide

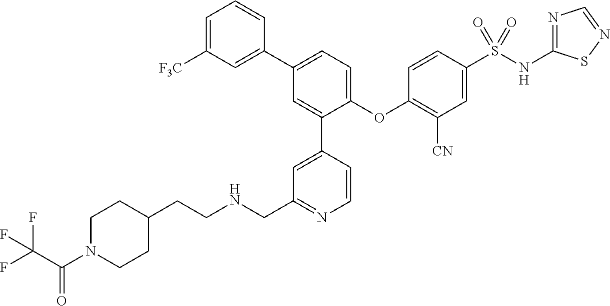 [SMILES: N#Cc1cc(S(=O)(=O)Nc2ncns2)ccc1Oc1ccc(-c2cccc(C(F)(F)F)c2)cc1-c1ccnc(CNCCC2CCN(C(=O)C(F)(F)F)CC2)c1]

To a solution of benzyl ({4-[4-{2-cyano-4-[(1,2,4-thiadiazol-5-ylamino)sulfonyl]phenoxy}-3′-(trifluoromethyl)biphenyl-3-yl]pyridin-2-yl}methyl){2-[1-(trifluoroacetyl)piperidin-4-yl]ethyl}carbamate (Preparation 7, 650 mg, 0.69 mmol) in acetic acid (10 mL) was added 48% HBr solution (5 mL). The reaction was heated at 50° C. for 10 minutes and then left stirring at room temperature for 72 hours. The solvent was evaporated in vacuo and the residue was azeotroped with methanol and purified by reverse phase column chromatography eluting with acetonitrile/water with 0.1% formic acid to afford the title compound as a white foam (140 mg, 25%).

¹H NMR (400 MHz, CDCl₃): δ ppm 1.13-1.22 (m, 2H), 1.76-1.82 (m, 5H), 2.67 (t, 1H), 3.06 (t, 1H), 3.25 (t, 2H), 3.94 (d, 1H), 4.48 (d, 1H), 4.45 (s, 2H), 6.75 (d, 1H), 7.45-7.48 (m, 2H), 7.61-7.70 (m, 4H), 7.77-7.81 (m, 2H), 7.85 (s, 1H), 7.91-7.95 (m, 2H), 8.06 (s, 1H), 8.54 (d, 1H).

¹⁹F NMR (400 MHz, CDCl₃): δ ppm −68.9 (s, CF₃), −62.6 (s, CF₃).

LCMS Rt=2.53 minutes, MS m/z 816 [MH]⁺

### EXAMPLE 26

### 4-({3-[2-(Aminomethyl)pyridin-4-yl]-3′-(trifluoromethyl)biphenyl-4-yl}oxy)-3-cyano-N-1,2,4-thiadiazol-5-ylbenzenesulfonamide bis-formate salt

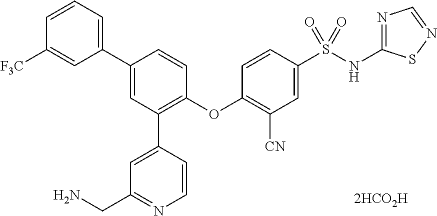 [SMILES: N#Cc1cc(S(=O)(=O)Nc2ncns2)ccc1Oc1ccc(-c2cccc(C(F)(F)F)c2)cc1-c1ccnc(CN)c1]

tert-Butyl ({4-[4-(2-cyano-4-{[(2,4-dimethoxybenzyl)(1,2,4-thiadiazol-5-yl)amino]sulfonyl}phenoxy)-3′-(trifluoromethyl)biphenyl-3-yl]pyridin-2-yl}methyl)carbamate (Preparation 24, 680 mg, 0.79 mmol) was dissolved in methanol (10 mL) and 12M hydrochloric acid (3.0 mL) was added. The reaction mixture was heated at 50° C. for 1 hour. The solvent was evaporated in vacuo and the residue was co-evaporated with methanol. The residue was purified by reverse phase chromatography (acetonitrile/water with 0.1% formic acid) to give the title compound as a white solid (350 mg, 63%).

¹H NMR (400 MHz, DMSO-d₆): δ ppm 4.21 (s, 2H), 6.94 (d, 1H), 7.52 (d, 1H), 7.62 (d, 1H), 7.71-7.78 (m, 3H), 7.86-7.90 (m, 2H), 7.96-8.01 (m, 3H), 8.09-8.10 (m, 2H), 8.23 (br. s, 2H), 8.63 (d, 1H).

¹⁹F NMR (400 MHz, DMSO-d₆): 5-60.9 (s).

LCMS Rt=2.79 minutes MS m/z 609 [M+H]⁺

### EXAMPLE 27

### N-[(4-{4-[2-chloro-5-fluoro-4-(1,3,4-thiadiazol-2-ylsulfamoyl)phenoxy]-3′-(trifluoromethyl)biphenyl-3-yl}pyridin-2-yl)methyl]-2,5,8,11,14,17,20,23,26,29,32,35-dodecaoxaoctatriacontan-38-amide

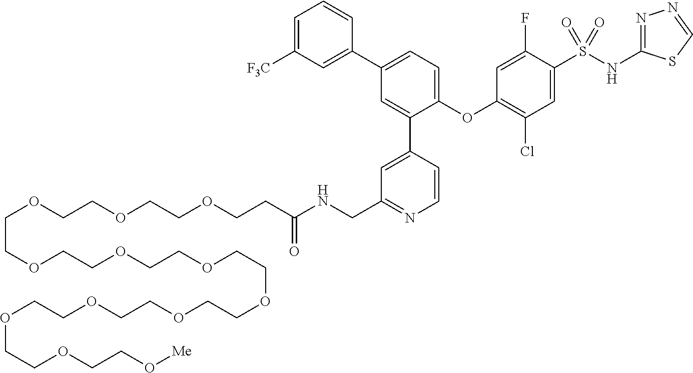 [SMILES: COCCOCCOCCOCCOCCOCCOCCOCCOCCOCCOCCOCCC(=O)NCc1cc(-c2cc(-c3cccc(C(F)(F)F)c3)ccc2Oc2cc(F)c(S(=O)(=O)Nc3nncs3)cc2Cl)ccn1]

The title compound was prepared according to the method described for Example 24 using 4-({3-[2-(aminomethyl)pyridin-4-yl]-3′-(trifluoromethyl)biphenyl-4-yl}oxy)-5-chloro-2-fluoro-N-1,3,4-thiadiazol-2-ylbenzenesulfonamide bis-formate (Example 7) and m-dPEG12-NHS ester.

LCMS Rt=3.00 minutes MS m/z 1206 [M+H]⁺

### EXAMPLE 28

### N-[(4-{4-[2-chloro-5-fluoro-4-(1,3-thiazol-4-ylsulfamoyl)phenoxy]-3′-(trifluoromethyl)biphenyl-3-yl}pyridin-2-yl)methyl]-2,5,8,11,14,17,20,23,26,29,32,35-dodecaoxaoctatriacontan-38-amide

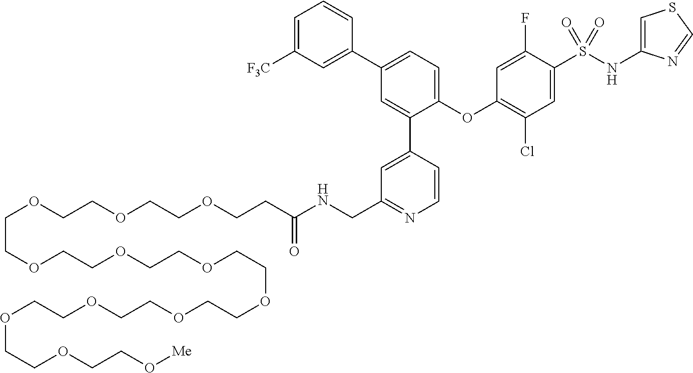 [SMILES: COCCOCCOCCOCCOCCOCCOCCOCCOCCOCCOCCOCCC(=O)NCc1cc(-c2cc(-c3cccc(C(F)(F)F)c3)ccc2Oc2cc(F)c(S(=O)(=O)Nc3cscn3)cc2Cl)ccn1]

The title compound was prepared according to the method described for Example 24 using 4-({3-[2-(aminomethyl)pyridin-4-yl]-3′-(trifluoromethyl)biphenyl-4-yl}oxy)-5-chloro-2-fluoro-N-(1,3-thiazol-4-yl)benzenesulfonamide (Example 29) and m-dPEG12-NHS ester.

LCMS Rt=2.58 minutes MS m/z 1204 [M+H]⁺

### EXAMPLE 29

### 4-({3-[2-(aminomethyl)pyridin-4-yl]-3′-(trifluoromethyl)biphenyl-4-yl}oxy)-5-chloro-2-fluoro-N-(1,3-thiazol-4-yl)benzenesulfonamide dihydrochloride salt

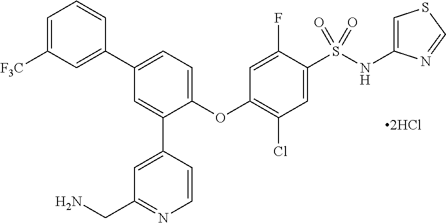 [SMILES: NCc1cc(-c2cc(-c3cccc(C(F)(F)F)c3)ccc2Oc2cc(F)c(S(=O)(=O)Nc3cscn3)cc2Cl)ccn1]

The title compound was prepared according to the methods described by Preparation 11 followed by Example 26 using tert-butyl 2-[(5-chloro-2,4-difluorophenyl)sulfonyl]-2-(thiazol-4-yl)acetate (WO2010079443) and tert-butyl ({4-[4-hydroxy-3′-(trifluoromethyl)biphenyl-3-yl]pyridin-2-yl}methyl)carbamate (Preparation 41) and isolated as the bis-hydrochloride salt.

¹H NMR (400 MHz, DMSO-d₆): δ ppm 4.20 (s, 2H), 6.20 (br s, 1H), 6.90 (m, 1H), 7.20 (m, 1H), 7.60-8.20 (m, 9H), 8.60 (s, 2H).

MS m/z 633 [M−H]⁻

### EXAMPLE 30

### 5-chloro-2-fluoro-4-{[3-{2-[({2-[1-(38-oxo-2,5,8,11,14,17,20,23,26,29,32,35-dodecaoxaoctatriacontan-38-yl)piperidin-4-yl]ethyl}amino)methyl]pyridin-4-yl}-3′-(trifluoromethyl)biphenyl-4-yl]oxy}-N-(1,3-thiazol-4-yl)benzenesulfonamide hydrochloride salt

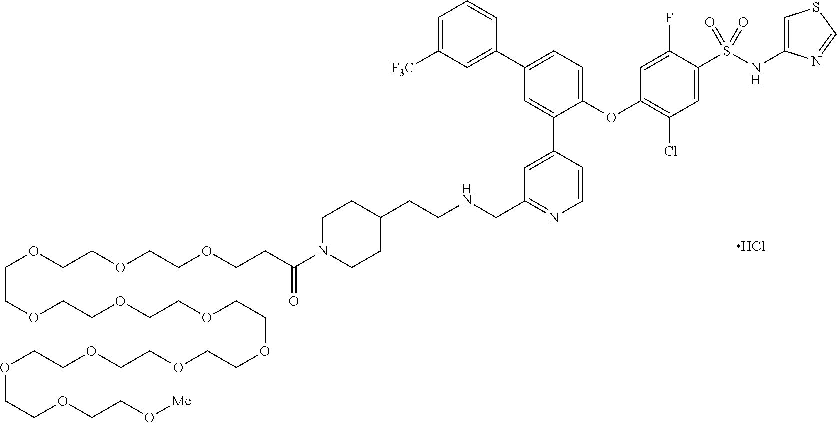 [SMILES: COCCOCCOCCOCCOCCOCCOCCOCCOCCOCCOCCOCCC(=O)N1CCC(CCNCc2cc(-c3cc(-c4cccc(C(F)(F)F)c4)ccc3Oc3cc(F)c(S(=O)(=O)Nc4cscn4)cc3Cl)ccn2)CC1 ; Cl]

The title compound was prepared according to the methods described by Example 24 followed by Example 1 using tert-butyl [(4-{4-[2-chloro-5-fluoro-4-(1,3-thiazol-4-ylsulfamoyl) phenoxy]-3′-(trifluoromethyl)biphenyl-3-yl}pyridin-2-yl)methyl][2-(piperidin-4-yl)ethyl]carbamate (Preparation 88) and m-dPEG12-NHS ester and isolated as the hydrochloride salt.

LCMS Rt=2.57 minutes MS m/z 1316 [M+H]⁺

### EXAMPLE 31

### 3-cyano-4-({3″-[({2-[1-(38-oxo-2,5,8,11,14,17,20,23,26,29,32,35-dodecaoxaoctatriacontan-38-yl)piperidin-4-yl]ethyl}amino)methyl]-1,1′:3′,1″-terphenyl-4′-yl}oxy)-N-(1,2,4-thiadiazol-5-yl)benzenesulfonamide

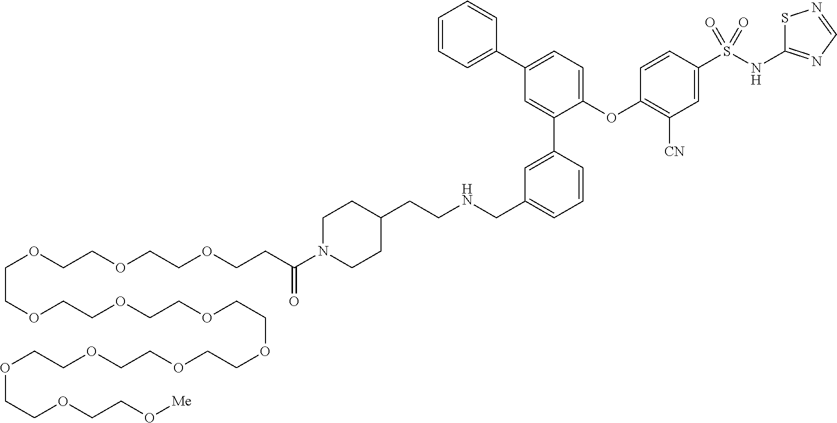 [SMILES: COCCOCCOCCOCCOCCOCCOCCOCCOCCOCCOCCOCCC(=O)N1CCC(CCNCc2cccc(-c3cc(-c4ccccc4)ccc3Oc3ccc(S(=O)(=O)Nc4ncns4)cc3C#N)c2)CC1]

The title compound was prepared according to the methods described by Example 24 followed by Example 5 with potassium carbonate using N-[(6′-{2-cyano-4-[(1,2,4-thiadiazol-5-ylamino)sulfonyl]phenoxy}-1,1′:3′,1″-terphenyl-3-yl)methyl]-2,2,2-trifluoro-N-(2-piperidin-4-ylethyl)acetamide (Preparation 14) and m-dPEG12-NHS ester.

Rt=2.32 minutes MS m/z 1219 [M−H]⁺

### EXAMPLE 32

### 5-chloro-2-fluoro-4-{[3-{2-[4-(38-oxo-2,5,8,11,14,17,20,23,26,29,32,35-dodecaoxaoctatriacontan-38-yl)piperazin-1-yl]pyridin-4-yl}-4′-(trifluoromethyl) biphenyl-4-yl]oxy}-N-(1,3,4-thiadiazol-2-yl)benzenesulfonamide

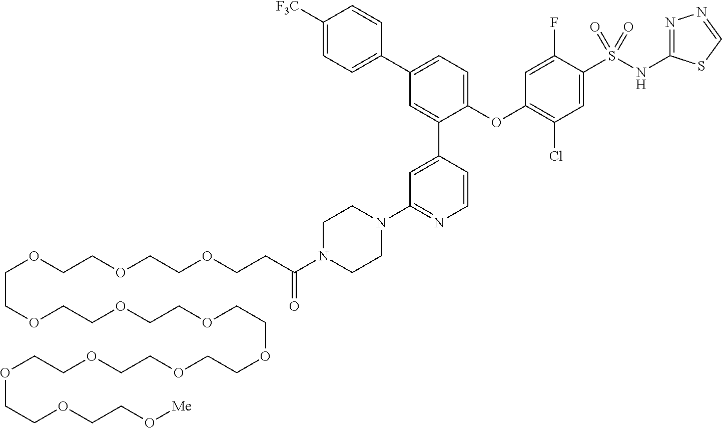 [SMILES: COCCOCCOCCOCCOCCOCCOCCOCCOCCOCCOCCOCCC(=O)N1CCN(c2cc(-c3cc(-c4ccc(C(F)(F)F)cc4)ccc3Oc3cc(F)c(S(=O)(=O)Nc4nncs4)cc3Cl)ccn2)CC1]

The title compound was prepared according to the method described for Example 24 using 5-chloro-2-fluoro-4-({3-[2-(piperazin-1-yl)pyridin-4-yl]-4′-(trifluoromethyl)biphenyl-4-yl}oxy)-N-(1,3,4-thiadiazol-2-yl)benzenesulfonamide (Preparation 87) and m-dPEG12-NHS ester.

LCMS Rt=3.26 minutes MS m/z 1261 [M+H]⁺

### EXAMPLE 33

### 5-chloro-2-fluoro-4-{[3-{2-[({2-[1-(38-oxo-2,5,8,11,14,17,20,23,26,29,32,35-dodecaoxaoctatriacontan-38-yl)piperidin-4-yl]ethyl}amino)methyl]pyridin-4-yl}-3′-(trifluoromethyl)biphenyl-4-yl]oxy}-N-(1,3,4-thiadiazol-2-yl)benzenesulfonamide formate salt

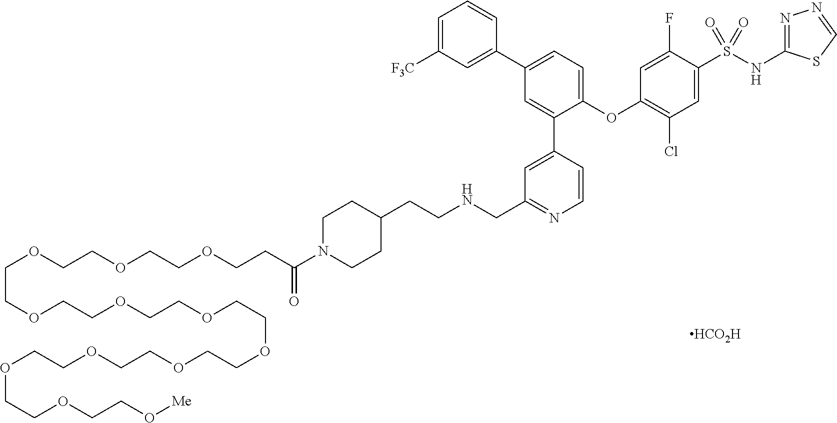 [SMILES: COCCOCCOCCOCCOCCOCCOCCOCCOCCOCCOCCOCCC(=O)N1CCC(CCNCc2cc(-c3cc(-c4cccc(C(F)(F)F)c4)ccc3Oc3cc(F)c(S(=O)(=O)Nc4nncs4)cc3Cl)ccn2)CC1 ; "·HCO2H"]

The title compound was prepared according to the method described for Example 17 using tert-butyl [(4-{4-[2-chloro-5-fluoro-4-(1,3,4-thiadiazol-2-ylsulfamoyl)phenoxy]-3′-(trifluoromethyl) biphenyl-3-yl}pyridin-2-yl)methyl][2-(piperidin-4-yl)ethyl]carbamate (Preparation 84) and m-dPEG12-NHS ester. The title compound was purified using preparative HPLC and isolated as the formate salt.

LCMS Rt=2.81 minutes MS m/z 1363 [MHCO₂H+H]⁺

### EXAMPLE 34

### 3-cyano-4-({3-[2-(2,5,8,11,14,17,20,23,26,29,32,35-dodecaoxa-38-azanonatriacontan-39-yl)pyridin-4-yl]-3′-(trifluoromethyl) biphenyl-4-yl}ox)-N-(1,3,4-thiadiazol-2-yl)benzenesulfonamide

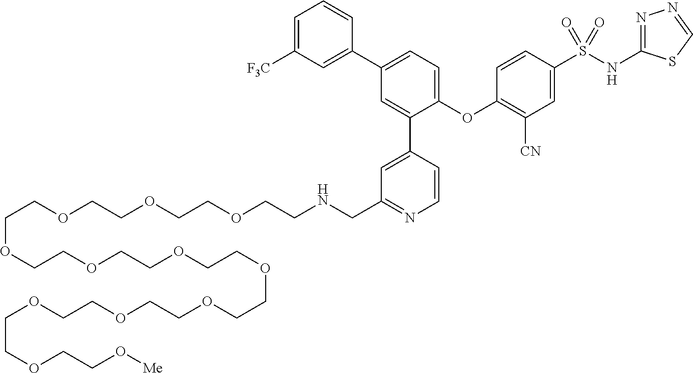 [SMILES: COCCOCCOCCOCCOCCOCCOCCOCCOCCOCCOCCOCCNCc1cc(-c2cc(-c3cccc(C(F)(F)F)c3)ccc2Oc2ccc(S(=O)(=O)Nc3nncs3)cc2C#N)ccn1]

The title compound was prepared according to the method described for Example 12 using tert-butyl (2,5,8,11,14,17,20,23,26,29,32,35-dodecaoxaheptatriacontan-37-yl)((4-(4-hydroxy-3′-(trifluoromethyl)-[1,1′-biphenyl]-3-yl)pyridin-2-yl)methyl)carbamate (Preparation 28) and 3-cyano-N-(2,4-dimethoxybenzyl)-4-fluoro-N-(1,3,4-thiadiazol-2-yl)benzenesulfonamide (WO2010079443).

LCMS Rt=1.64 minutes MS m/z 1149 [M−H]⁻

### EXAMPLE 35

### 5-chloro-2-fluoro-N-(1,3-thiazol-4-yl)-4-{[3-{2-[({2-[1-(trifluoroacetyl)piperidin-4-yl]ethyl}amino)methyl]pyridin-4-yl}-3′-(trifluoromethyl)biphenyl-4-yl]oxy}benzenesulfonamide

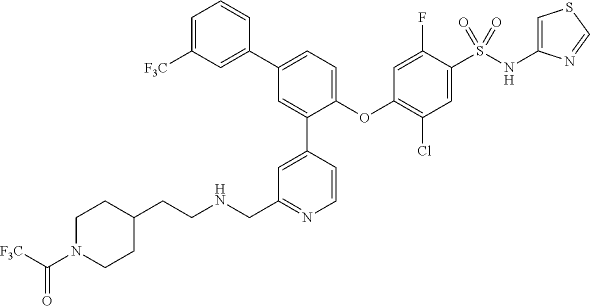 [SMILES: O=C(N1CCC(CCNCc2cc(-c3cc(-c4cccc(C(F)(F)F)c4)ccc3Oc3cc(F)c(S(=O)(=O)Nc4cscn4)cc3Cl)ccn2)CC1)C(F)(F)F]

The title compound was prepared according to the methods described for Preparation 15 followed by Example 2 using 2,2,2-trichloroethyl ({4-[4-{2-chloro-5-fluoro-4-[(1,3-thiazol-4-ylamino)sulfonyl]phenoxy}-3′-(trifluoromethyl) biphenyl-3-yl]pyridin-2-yl}methyl)(2-piperidin-4-ylethyl)carbamate hydrochloride salt (Preparation 10).

¹H NMR (400 MHz, CDCl₃): δ ppm 1.10 (m, 2H), 1.70 (m, 2H), 1.80 (m, 2H), 2.65 (t, 1H), 2.90 (t, 1H), 3.05 (m, 1H), 3.20-3.80 (br m, 2H), 3.95 (d, 1H), 4.05 (s, 2H), 4.45 (d, 1H), 6.35 (d, 1H), 7.00 (s, 1H), 7.40 (d, 1H), 7.50 (s, 1H), 7.60-7.75 (m, 4H), 7.78 (d, 2H), 7.80 (s, 1H), 8.30 (s, 1H), 8.50 (d, 1H), 8.65 (s, 1H).

MS m/z 842 [M+H]⁺

### Preparation 1

### tert-Butyl 4-{2-[({4-[4-{2-chloro-5-fluoro-4-[(1,3,4-thiadiazol-2-ylamino)sulfonyl]phenoxy}-3′-(trifluoromethyl)biphenyl-3-yl]pyridin-2-yl}methyl)amino]ethyl}piperidine-1-carboxylate bis-formate salt

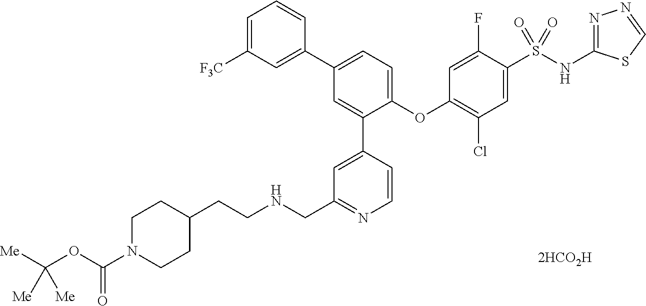 [SMILES: CC(C)(C)OC(=O)N1CCC(CCNCc2cc(-c3cc(-c4cccc(C(F)(F)F)c4)ccc3Oc3cc(F)c(S(=O)(=O)Nc4nncs4)cc3Cl)ccn2)CC1]

To a suspension of 4-({3-[2-(aminomethyl)pyridin-4-yl]-3′-(trifluoromethyl)biphenyl-4-yl}oxy)-5-chloro-2-fluoro-N-1,3,4-thiadiazol-2-ylbenzenesulfonamide bis-formate salt (Example 7, 50 mg, 0.069 mmol) in methanol (2 mL), was added triethylamine (30 μL, 0.207 mmol), followed by tert-butyl 4-(2-oxoethyl)piperidine-1-carboxylate (23.4 mg, 0.103 mmol). The reaction was stirred at room temperature for 18 hours and sodium borohydride (16 mg, 0.414 mmol) was added. After 30 minutes at room temperature, the mixture was quenched by the addition of water (5 mL). The organic phase was extracted with ethyl acetate (3×5 mL) and the combined organic layers were washed with brine (10 mL), dried over anhydrous magnesium sulfate, filtered and concentrated in vacuo. The resulting residue was purified by reverse phase column chromatography eluting with 5-95% acetonitrile in water with 0.1% formic acid) to afford the title compound (38 mg, 67%).

¹H NMR (400 MHz, MeOD-d₄): δ ppm 1.05 (m, 2H), 1.40 (s, 9H), 1.60-1.70 (m, 5H), 2.70 (m, 2H), 3.15 (d, 2H), 4.05 (d, 2H), 4.40 (s, 2H), 6.50 (d, 1H), 7.30 (d, 1H), 7.60-7.75 (m, 4H), 7.80 (m, 3H), 7.95 (m, 2H), 8.15 (br s, 1H), 8.60 (m, 2H).

¹⁹F NMR (400 MHz, MeOD d₄): δ ppm −63.0 (s, 3F), −108.0 (s, 1F).

LCMS Rt=2.67 minutes MS m/z 847 [M+H]⁺

### Preparation 2

### 3-cyano-4-((3-(2-(piperazin-1-yl)pyridin-4-yl)-4′-(trifluoromethyl)-[1,1′-biphenyl]-4-yl)oxy)-N-(1,2,4-thiadiazol-5-yl)benzenesulfonamide bis-hydrochloride salt

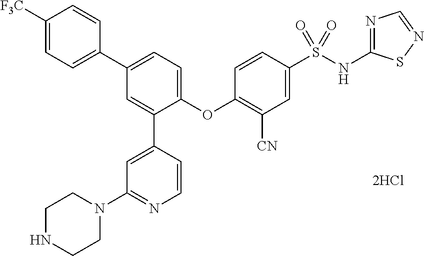 [SMILES: N#Cc1cc(S(=O)(=O)Nc2ncns2)ccc1Oc1ccc(-c2ccc(C(F)(F)F)cc2)cc1-c1ccnc(N2CCNCC2)c1]

To a solution of tert-butyl 4-(4-(4-(2-cyano-4-(N-(2,4-dimethoxybenzyl)-N-(1,2,4-thiadiazol-5-yl)sulfamoyl)phenoxy)-4′-(trifluoromethyl)-[1,1′-biphenyl]-3-yl)pyridin-2-yl)piperazine-1-carboxylate (Preparation 9, 1.12 g, 1.22 mmol) in DCM (15 mL) was added 4M HCl in dioxane (10 mL) followed by MeOH (2 mL). The reaction was stirred at room temperature for 18 hours. The resulting precipitate was filtered and the filtrate concentrated in vacuo. The residue was triturated with DCM and EtOAc to afford a white solid that was filtered and dried to afford the title compound.

¹H NMR (400 MHz, DMSO-d₆): δ ppm 3.20 (m, 4H), 3.80 (m, 4H), 6.55 (m, 1H), 7.00 (m, 1H), 7.12 (m, 1H), 7.55 (d, 1H), 7.86 (m, 2H), 7.95 (m, 3H), 8.00 (m, 2H), 8.10 (m, 1H), 9.02 (br s, 1H).

MS m/z 664 [M+H]⁺

### Preparation 3

### tert-Butyl ({4-[4-(2-chloro-4-{[(2,4-dimethoxybenzyl)(1,3,4-thiadiazol-2-yl)amino]sulfonyl}-5-fluorophenoxy)-3′-(trifluoromethyl)biphenyl-3-yl]pyridin-2-yl}methyl)carbamate

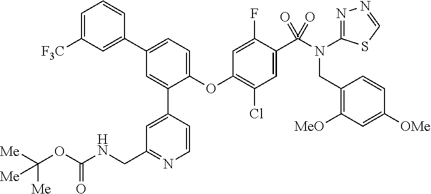

To a solution of tert-Butyl ({4-[4-hydroxy-3′-(trifluoromethyl)biphenyl-3-yl]pyridin-2-yl}methyl)carbamate (Preparation 41, 708 mg, 1.59 mmol) in DMSO (15 mL) was added 5-chloro-N-(2,4-dimethoxybenzyl)-2,4-difluoro-N-(1,3,4-thiadiazol-2-yl)benzenesulfonamide (WO2010079443, 735 mg, 1.59 mmol) followed by potassium carbonate (660 mg, 4.78 mmol). The reaction was stirred at room temperature for 18 hours. Water (20 mL) was added and the organic phase was extracted with ethyl acetate (3×20 mL). The combined organic layers were washed with brine (30 mL), dried over anhydrous magnesium sulfate and concentrated in vacuo. The residue was purified by silica gel column chromatography eluting with 7-60% EtOAc in heptanes to afford the title compound as a light yellow solid (732 mg, 52%).

¹H NMR (400 MHz, CDCl₃): δ ppm 1.40 (s, 9H), 3.60 (s, 3H), 3.75 (s, 3H), 4.45 (m, 2H), 5.20 (s, 2H), 5.60 (br s, 1H), 6.20 (s, 1H), 6.35 (m, 2H), 7.20 (m, 2H), 7.35 (d, 1H), 7.50 (s, 1H), 7.60-7.80 (m, 6H), 7.85 (s, 1H), 8.55 (s, 1H), 8.80 (s, 1H).

¹⁹F NMR (400 MHz, CDCl₃): δ ppm −63.0 (s, 3F), −105.0 (s, 1F).

LCMS Rt=3.24 minutes MS m/z 886 [M+H]⁺

### Preparation 4

### tert-Butyl [2-(1-acetylpiperidin-4-yl)ethyl]({4-[4-{2-cyano-4-[(1,2,4-thiadiazol-5-ylamino)sulfonyl]phenoxy}-3′-(trifluoromethyl)biphenyl-3-yl]pyridin-2-yl}methyl)carbamate

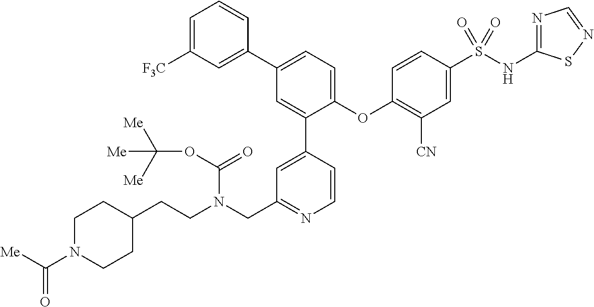 [SMILES: CC(=O)N1CCC(CCN(Cc2cc(-c3cc(-c4cccc(C(F)(F)F)c4)ccc3Oc3ccc(S(=O)(=O)Nc4ncns4)cc3C#N)ccn2)C(=O)OC(C)(C)C)CC1]

Triethylamine (32 μL, 0.23 mmol) and acetic anhydride (11 μL, 0.11 mmol) were added to a solution of tert-butyl {[4-(3′-tert-butyl-4-{2-cyano-4-[(1,2,4-thiadiazol-5-ylamino)sulfonyl]phenoxy}biphenyl-3-yl)pyridin-2-yl]methyl}(2-piperidin-4-ylethyl)carbamate (Preparation 5, 41 mg, 0.06 mmol) in dichloromethane (1 mL). The reaction mixture was stirred for 2 hours at room temperature, washed with saturated sodium hydrogen carbonate. The organic layer was separated and dried over magnesium sulfate. The filtrate was evaporated under reduced pressure to afford the title compound (49 mg, 100%).

¹H NMR (400 MHz, CDCl₃): δ ppm 1.05-1.13 (m, 2H), 1.43-1.49 (m, 11H), 1.64-1.68 (m, 3H), 2.06 (s, 3H), 2.49 (t, 1H), 2.99 (t, 1H), 3.24-3.36 (m, 1H), 3.74 (d, 1H), 4.52 (d, 4H), 6.75 (d, 1H), 7.21-7.24 (m, 1H), 7.41 (d, 1H), 7.58-7.69 (m, 5H), 7.77 (d, 1H), 7.82 (s, 1H), 7.95-7.97 (m, 2H), 8.06 (d, 1H), 8.55 (d, 1H).

¹⁹F NMR (400 MHz, CDCl₃): δ ppm −62.6 (s, 3F).

LCMS Rt=2.77 minutes MS m/z 862 [M+H]⁺

### Preparation 5

### tert-Butyl {[4-(3′-tert-butyl-4-{2-cyano-4-[(1,2,4-thiadiazol-5-ylamino)sulfonyl]phenoxy}biphenyl-3-yl)pyridin-2-yl]methyl}(2-piperidin-4-ylethyl)carbamate

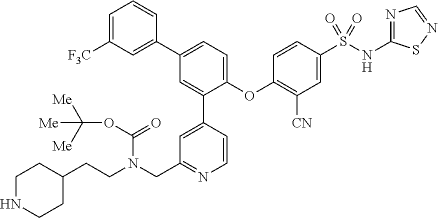 [SMILES: CC(C)(C)OC(=O)N(CCC1CCNCC1)Cc1cc(-c2cc(-c3cccc(C(F)(F)F)c3)ccc2Oc2ccc(S(=O)(=O)Nc3ncns3)cc2C#N)ccn1]

tert-Butyl ({4-[4-{2-cyano-4-[(1,2,4-thiadiazol-5-ylamino)sulfonyl]phenoxy}-3′-(trifluoromethyl)biphenyl-3-yl]pyridin-2-yl}methyl){2-[1-(trifluoroacetyl)piperidin-4-yl]ethyl}carbamate (Preparation 6, 124 mg, 0.14 mmol) was dissolved in 7M ammonia in methanol (4 mL) and the mixture stirred at room temperature for 18 hours. The solvent was evaporated in vacuo and the residue was dissolved in methanol (2.0 mL) and purified by SCX cartridge (1 g) eluting first with methanol (10 mL) and then with a solution of 7M ammonia in methanol (10 mL) to afford the title compound as a yellow foam (91 mg, 82%).

¹H NMR (400 MHz, CDCl₃): δ ppm 0.96 (br. s, 4H), 1.34-2.69 (m, 12H), 2.79 (t, 2H), 3.08-3.20 (m, 2H), 3.45 (br s, 2H), 4.55 (br s, 2H), 6.52 (d, 1H), 7.26-7.34 (m, 2H), 7.49-7.89 (m, 9H), 8.04 (s, 1H), 8.55 (d, 1H).

¹⁹F NMR (400 MHz, CDCl₃): δ ppm −62.6 (s, 3F).

LCMS Rt=2.52 minutes MS m/z 820 [M+H]⁺

### Preparation 6

### tert-Butyl ({4-[4-{2-cyano-4-[(1,2,4-thiadiazol-5-ylamino)sulfonyl]phenoxy}-3′-(trifluoromethyl)biphenyl-3-yl]pyridin-2-yl}methyl){2-[1-(trifluoroacetyl)piperidin-4-yl]ethyl}carbamate

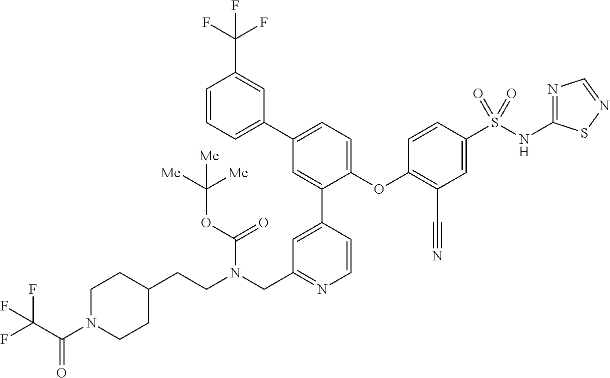 [SMILES: CC(C)(C)OC(=O)N(CCC1CCN(C(=O)C(F)(F)F)CC1)Cc1cc(-c2cc(-c3cccc(C(F)(F)F)c3)ccc2Oc2ccc(S(=O)(=O)Nc3ncns3)cc2C#N)ccn1]

Di-tert-butyl dicarbonate (44 mg, 0.20 mmol) and triethylamine (70 μL, 0.50 mmol) were added to a solution of 3-cyano-N-1,2,4-thiadiazol-5-yl-4-{[3-{2-[({2-[1-(trifluoroacetyl)piperidin-4-yl]ethyl}amino)methyl]pyridin-4-yl}-3′-(trifluoromethyl)biphenyl-4-yl]oxy}benzenesulfonamide (Example 25, 137 mg, 0.16 mmol) in dichloromethane (3 mL) and the reaction mixture was stirred for 1 hour. Then mixture was diluted with dichloromethane (20 mL), washed with water (10 mL). The organic layer was dried over magnesium sulfate and concentrated in vacuo. The residue was purified by silica gel column chromatography eluting with 15% MeOH in DCM to afford the title compound as white foam (124 mg, 88%).

¹H NMR (400 MHz, CDCl₃): δ ppm 1.06-1.16 (m, 2H), 1.36-1.42 (m, 12H), 1.62-1.77 (m, 2H), 2.65 (br s, 1H), 3.03 (br s, 1H), 3.22 (br s, 2H), 3.91 (br s, 1H), 4.34-4.59 (m, 3H), 6.73 (d, 1H), 7.29-8.04 (m, 12H), 8.58 (d, 1H).

¹⁹F NMR (400 MHz, CDCl₃): δ ppm −68.9 (s, 3F), −62.6 (s, 3F).

LCMS Rt=3.02 minutes MS m/z 916 [M+H]⁺

### Preparation 7

### Benzyl ({4-[4-{2-cyano-4-[(1,2,4-thiadiazol-5-ylamino)sulfonyl]phenoxy}-3′-(trifluoromethyl)biphenyl-3-yl]pyridin-2-yl}methyl){2-[1-(trifluoroacetyl)piperidin-4-yl]ethyl}carbamate

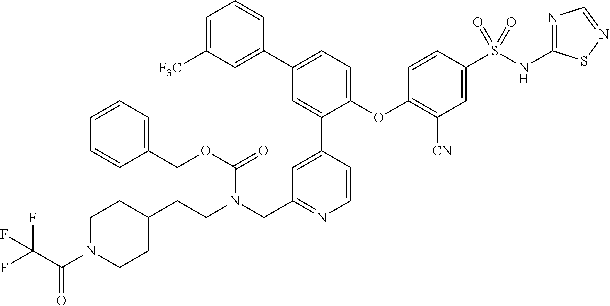 [SMILES: N#Cc1cc(S(=O)(=O)Nc2ncns2)ccc1Oc1ccc(-c2cccc(C(F)(F)F)c2)cc1-c1ccnc(CN(CCC2CCN(C(=O)C(F)(F)F)CC2)C(=O)OCc2ccccc2)c1]

tert-Butyl 4-(2-{[(benzyloxy)carbonyl]({4-[4-(2-cyano-4-{[(2,4-dimethoxybenzyl)(1,2,4-thiadiazol-5-yl)amino]sulfonyl}phenoxy)-3′-(trifluoromethyl)biphenyl-3-yl]pyridin-2-yl}methyl)amino}ethyl)piperidine-1-carboxylate (Preparation 8, 1.96 g, 1.78 mmol) was dissolved in dioxane (20 mL) and 4M HCl in dioxane (3.6 mL) was added. The reaction mixture was stirred at room temperature for 4 hours. The solvent was evaporated and the residue suspended in dichloromethane (15 mL). Triethylamine (1 mL, 7.12 mmol) and trifluoroacetic anhydride (0.26 mL, 1.87 mmol) were added and mixture was stirred at room temperature for 1 hour. The reaction mixture was diluted with dichloromethane (100 mL), washed with saturated sodium hydrogen carbonate, the organic layer was dried over magnesium sulfate and the filtrate was evaporated in vacuo. The residue was purified by reverse phase column chromatography eluting with acetonitrile/water both with 0.1% formic acid to give the title compound as a yellow foam (650 mg, 38%).

¹H NMR (400 MHz, CDCl₃): δ ppm 0.88-1.88 (m, 5H), 2.54-2.73 (m, 1H), 2.88-3.11 (m, 1H), 3.30 (br s, 2H), 3.62-3.99 (m, 3H), 4.33-4.72 (m, 3H), 5.19 (d, 2H), 6.61-6.71 (m, 1H), 7.17-7.42 (m, 7H), 7.61-7.85 (m, 9H), 8.02 (d, 1H), 8.50-8.56 (m, 1H).

¹⁹F NMR (400 MHz, CDCl₃): δ ppm −68.9 (s, 3F), −62.6 (s, 3F).

LCMS Rt=3.02 minutes MS m/z 950 [M+H]⁺

### Preparation 8

### tert-Butyl 4-(2-{[(benzyloxy)carbonyl]({4-[4-(2-cyano-4-{[(2,4-dimethoxybenzyl)(1,2,4-thiadiazol-5-yl)amino]sulfonyl}phenoxy)-3′-(trifluoromethyl)biphenyl-3-yl]pyridin-2-yl}methyl)amino}ethyl)piperidine-1-carboxylate

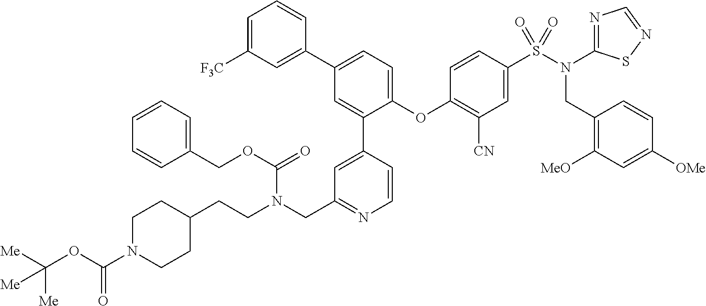 [SMILES: COc1ccc(CN(c2ncns2)S(=O)(=O)c2ccc(Oc3ccc(-c4cccc(C(F)(F)F)c4)cc3-c3ccnc(CN(CCC4CCN(C(=O)OC(C)(C)C)CC4)C(=O)OCc4ccccc4)c3)c(C#N)c2)c(OC)c1]

tert-Butyl 4-(2-{[(benzyloxy)carbonyl]({4-[4-hydroxy-3′-(trifluoromethyl)biphenyl-3-yl]pyridin-2-yl}methyl)amino}ethyl)piperidine-1-carboxylate (Preparation 25, 1.50 g, 2.18 mmol) was dissolved in dimethyl sulfoxide (20 mL) and potassium carbonate (0.60 g, 4.35 mmol) followed by 3-cyano-N-(2,4-dimethoxybenzyl)-4-fluoro-N-(1,2,4-thiadiazol-5-yl)benzenesulfonamide (WO2010079443, 0.95 g, 2.18 mmol) were added. The reaction was stirred at room temperature for 1 hour. The reaction was partitioned between ethyl acetate (100 mL) and water (50 mL). The organic layer was dried over magnesium sulfate and concentrated in vacuo to afford the title compound as brown foam (2.16 g, 90%).

¹H NMR (400 MHz, CDCl₃): δ ppm 1.00-1.10 (m, 2H), 1.37-1.64 (m, 14H), 2.61 (br s, 2H), 3.35 (br s, 2H), 3.55 (s, 3H), 3.81 (s, 3H), 4.00 (br s, 2H), 4.59 (s, 2H), 5.12 (d, 2H), 5.27 (s, 2H), 6.18 (d, 1H), 6.35 (dd, 1H), 6.56 (dd, 1H), 7.06-7.12 (m, 3H), 7.22-7.41 (m, 6H), 7.56-7.84 (m, 8H), 8.18 (s, 1H), 8.56 (d, 1H).

¹⁹F NMR (400 MHz, CDCl₃): δ ppm −62.7 (s, 3F).

LCMS Rt=3.43 minutes MS m/z No mass ion observed

### Preparation 9

### tert-butyl 4-(4-(4-(2-cyano-4-(N-(2,4-dimethoxybenzyl)-N-(1,2,4-thiadiazol-5-yl)sulfamoyl)phenoxy)-4′-(trifluoromethyl)-[1,1′-biphenyl]-3-yl)pyridin-2-yl)piperazine-1-carboxylate

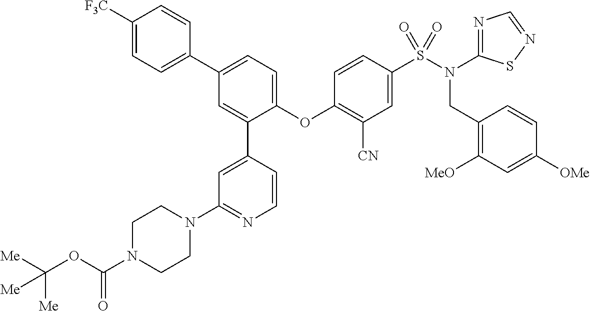 [SMILES: COc1ccc(CN(c2ncns2)S(=O)(=O)c2ccc(Oc3ccc(-c4ccc(C(F)(F)F)cc4)cc3-c3ccnc(N4CCN(C(=O)OC(C)(C)C)CC4)c3)c(C#N)c2)c(OC)c1]

The title compound was prepared according to the method described for Preparation 8 using 3-cyano-N-(2,4-dimethoxybenzyl)-4-fluoro-N-(1,2,4-thiadiazol-5-yl)benzenesulfonamide (WO2010079443) and tert-butyl 4-(4-(4-hydroxy-4′-(trifluoromethyl)-[1,1′-biphenyl]-3-yl)pyridin-2-yl)piperazine-1-carboxylate (WO2012004743). The reaction was quenched by the addition of water and the resulting precipitate filtered and dried.

¹H NMR (400 MHz, DMSO-d₆): δ ppm 1.40 (s, 9H), 3.40-3.55 (m, 8H), 3.63 (s, 3H), 3.78 (s, 3H), 5.20 (s, 2H), 6.37 (m, 1H), 6.45 (m, 1H), 6.78 (m, 2H), 6.95 (m, 2H), 7.00 (d, 1H), 7.55 (d, 1H), 7.90 (m, 2H), 7.95-8.15 (m, 7H).

MS m/z 914 [M+H]⁺

### Preparation 10

### 2,2,2-Trichloroethyl ({4-[4-{2-chloro-5-fluoro-4-[(1, 3-thiazol-4-ylamino)sulfonyl]phenoxy}-3′-(trifluoromethyl)biphenyl-3-yl]pyridin-2-yl}methyl)(2-piperidin-4-ylethyl)carbamate hydrochloride salt

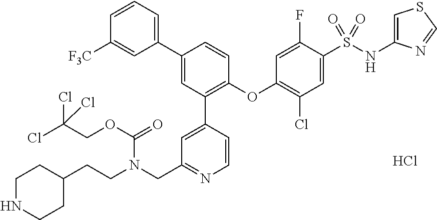 [SMILES: O=C(OCC(Cl)(Cl)Cl)N(CCC1CCNCC1)Cc1cc(-c2cc(-c3cccc(C(F)(F)F)c3)ccc2Oc2cc(F)c(S(=O)(=O)Nc3cscn3)cc2Cl)ccn1 ; Cl]

To a solution of tert-butyl 4-(2-{({4-[4-(4-{[(tert-butoxycarbonyl)(1,3-thiazol-4-yl)amino]sulfonyl}-2-chloro-5-fluorophenoxy)-3′-(trifluoromethyl)biphenyl-3-yl]pyridin-2-yl}methyl)[(2,2,2-trichloroethoxy)carbonyl]amino}ethyl)piperidine-1-carboxylate (Preparation 11, 2.47 g, 2.20 mmol) in 1,4-dioxane (10 mL) was added a solution of hydrogen chloride in 1,4-dioxane (4M, 5.5 mL, 22 mmol). The reaction was stirred at room temperature for 18 hours and concentrated in vacuo to afford the title compound as a light yellow foam (2.15 g, 100%).

¹H NMR (400 MHz, MeOD-d₄): δ ppm 1.40 (m, 2H), 1.60 (m, 3H), 1.95 (m, 2H), 2.95 (dd, 2H), 3.30 (d, 2H), 3.60 (m, 2H), 4.80 (s, 2H), 5.00 (d, 2H), 7.03 (d, 1H), 7.05 (s, 1H), 7.25 (m, 1H), 7.70 (m, 2H), 7.95-8.05 (m, 6H), 8.20 (m, 1H), 8.30 (d, 1H), 8.75 (s, 1H), 8.80 (m, 1H).

¹⁹F NMR (400 MHz, MeOD-d₄): δ ppm −64.0 (s, 3F), −108.0 (s, 1F).

LCMS Rt=2.98 minutes MS m/z 920 [M+H]⁺

### Preparation 11

### tert-Butyl 4-(2-{({4-[4-(4-{[(tert-butoxycarbonyl)(1,3-thiazol-4-yl)amino]sulfonyl}-2-chloro-5-fluorophenoxy)-3′-(trifluoromethyl)biphenyl-3-yl]pyridin-2-yl}methyl)[(2,2,2-trichloroethoxy)carbonyl]amino}ethyl)piperidine-1-carboxylate

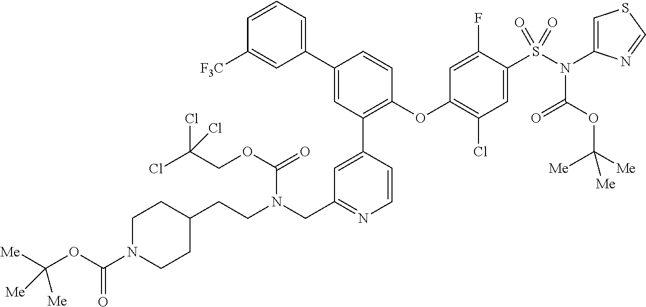 [SMILES: CC(C)(C)OC(=O)N1CCC(CCN(Cc2cc(-c3cc(-c4cccc(C(F)(F)F)c4)ccc3Oc3cc(F)c(S(=O)(=O)N(C(=O)OC(C)(C)C)c4cscn4)cc3Cl)ccn2)C(=O)OCC(Cl)(Cl)Cl)CC1]

To a solution of tert-butyl 4-(2-{({4-[4-hydroxy-3′-(trifluoromethyl)biphenyl-3-yl]pyridin-2-yl}methyl)[(2,2,2-trichloroethoxy)carbonyl]amino}ethyl)piperidine-1-carboxylate (Preparation 26, 2.0 g, 2.73 mmol) in DMSO (15 mL) was added tert-butyl 2-[(5-chloro-2,4-difluorophenyl)sulfonyl]-2-(thiazol-4-yl)acetate (WO2010079443, 1.21 g, 2.73 mmol) followed by potassium carbonate (1.13 g, 8.19 mmol). The reaction was stirred at room temperature for 2 hours. Water (20 mL) was added and the aqueous phase was extracted with ethyl acetate (3×20 mL). The combined organic layers were washed with brine (30 mL), dried over anhydrous magnesium sulfate and concentrated in vacuo.

The residue was purified by silica gel column chromatography eluting with cyclohexane:ethyl acetate eluting with 10-80% EtOAc in cyclohexanes to afford the title compound as a light yellow foam (2.47 g, 81%).

¹H NMR (400 MHz, CDCl₃): δ ppm 1.10 (m, 2H), 1.30 (s, 9H), 1.40 (m, 2H), 1.45 (s, 9H) 1.60 (m, 3H), 2.50 (m, 2H), 3.40 (dd, 2H), 4.00 (m, 2H), 4.60 (m, 2H), 4.70 (d, 2H), 6.45 (d, 1H), 7.20 (m, 1H), 7.40 (m, 2H), 7.50 (s, 1H), 7.55 (m, 1H), 7.58 (m, 1H), 7.60-7.75 (m, 2H), 7.78 (d, 1H), 7.80 (s, 1H), 8.05 (d, 1H), 8.55 (d, 1H), 8.80 (d, 1H).

¹⁹F NMR (400 MHz, CDCl₃): δ ppm −63.0 (s, 3F), −105.0 (s, 1F).

LCMS Rt=3.52 minutes MS m/z 1120 [M+H]⁺

### Preparation 12

### tert-Butyl (2-{4-[(6′-{2-cyano-4-[(1,2,4-thiadiazol-5-ylamino)sulfonyl]phenoxy}-1,1′:3′,1″-terphenyl-3-yl)methyl]piperazin-1-yl}ethyl)carbamate

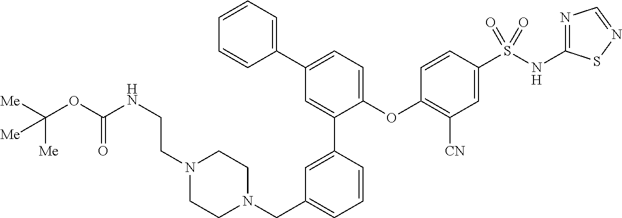 [SMILES: CC(C)(C)OC(=O)NCCN1CCN(Cc2cccc(-c3cc(-c4ccccc4)ccc3Oc3ccc(S(=O)(=O)Nc4ncns4)cc3C#N)c2)CC1]

To a solution of 3-cyano-4-[(3″-formyl-1,1′:3′,1″-terphenyl-4′-yl)oxy]-N-1,2,4-thiadiazol-5-ylbenzenesulfonamide (Preparation 13, 700 mg, 1.3 mmol) in dichloromethane (10 mL) was added acetic acid (0.075 mL, 1.3 mmol) and tert-butyl (2-piperazin-1-ylethyl)carbamate (310 mg, 1.36 mmol) and stirred for 30 minutes at room temperature. Sodium triacetoxyborohydride (282 mg, 1.50 mmol) was added and the mixture stirred at room temperature for 18 hours. Water (5 mL) was added and the resulting mixture extracted with ethyl acetate (50 mL). The organic layer was washed with brine (50 mL) and dried over magnesium sulfate, filtered and concentrated in vacuo. The product was purified by silica gel column chromatography eluting with 2-20% MeOH in EtOAc to afford the title compound (600 mg, 61%).

¹H NMR (400 MHz, MeOD-d₄): δ ppm 1.40 (s, 9H), 2.50 (s, 4H), 3.1 (m, 2H), 3.20 (m, 2H), 3.40 (m, 2H), 3.45 (m, 2H), 3.55 (m, 2H), 6.70 (m, 1H), 7.20-7.50 (m, 8H), 7.70 (m, 3H), 7.80 (m, 2H), 8.00 (m, 2H).

LCMS Rt=3.09 minutes MS m/z 752 [M+H]⁺

### Preparation 13

### 3-Cyano-4-[(3″-formyl-1,1′:3′,1″-terphenyl-4′-yl)oxy]-N-1,2,4-thiadiazol-5-ylbenzenesulfonamide

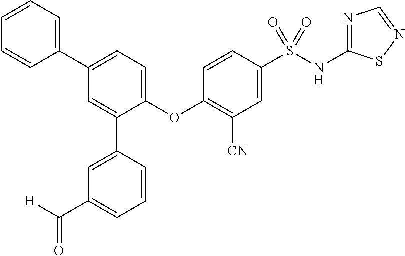 [SMILES: N#Cc1cc(S(=O)(=O)Nc2ncns2)ccc1Oc1ccc(-c2ccccc2)cc1-c1cccc(C=O)c1]

To a solution of 6′-hydroxy-[1,1′:3′,1″-terphenyl]-3-carbaldehyde (Preparation 38, 507 mg, 1.85 mmol) in DMSO (15 mL) was added 3-cyano-4-fluoro-N-1,2,4-thiadiazol-5-ylbenzenesulfonamide (WO2010079443, 500 mg, 1.76 mmol) and K₂CO₃ (972 mg, 7.04 mmol). The mixture was heated at 80° C. for 2 hours and then diluted with brine (50 mL). The mixture was extracted with EtOAc (30 mL) and the organic layer was washed with brine (50 mL), dried over magnesium sulfate, filtered and concentrated in vacuo. The residue was purified by reverse phase column chromatography eluting with (mobile phase A: 0.1% formic acid in water, mobile phase B: 0.1% formic acid in acetonitrile, gradient from 0% to 40% of B) to afford the title compound as an off-white solid (700 mg, 74%).

¹H NMR (400 MHz, DMSO-d₆): δ ppm 7.00 (m, 1H), 7.40 (m, 1H), 7.50 (m, 3H), 7.60 (m, 1H), 7.80-7.90 (m, 7H), 8.10 (m, 2H), 8.40 (s, 1H), 10.00 (s, 1H).

LCMS Rt=4.25 minutes MS m/z 539 [M+H]⁺

### Preparation 14

### N-[(6′-{2-cyano-4-[(1,2,4-thiadiazol-5-ylamino)sulfonyl]phenoxy}-1,1′:3′,1″-terphenyl-3-yl)methyl]-2,2,2-trifluoro-N-(2-piperidin-4-ylethyl)acetamide trifluoroacetate salt

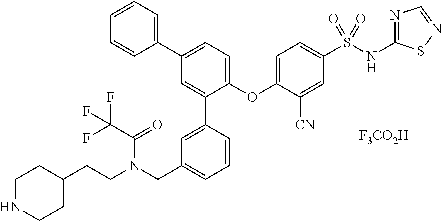

Trifluoroacetic acid (0.88 mL, 11.5 mmol) was added to a solution of tert-butyl 4-{2-[{[6′-(2-cyano-4-{[(2,4-dimethoxybenzyl)(1,2,4-thiadiazol-5-yl)amino]sulfonyl}phenoxy)-1,1′:3′,1″-terphenyl-3-yl]methyl}(trifluoroacetyl)amino]ethyl}piperidine-1-carboxylate (Preparation 15, 1149 mg, 1.15 mmol) in dichloromethane (32 mL) which was stirred for 18 hours at room temperature under nitrogen. The reaction was quenched by the addition of methanol (20 mL) which was passed through a pad of Arbocel® and washed with addition methanol (100 mL). The organic filtrate was concentrated in vacuo and the residue was purified using silica gel column chromatography eluting with 1-20% methanol:dichloromethane to afford the title compound as a colourless solid (856 mg, 86%).

¹H NMR (400 MHz, CDCl₃) δ ppm 1.11-1.24 (m, 2H), 1.32-1.49 (m, 3H), 1.66-1.70 (m, 2H), 2.77-2.87 (m, 2H), 3.13-3.16 (m, 1H), 3.16-3.18 (m 3H), 4.64 (s, 1H), 4.67 (s, 1H), 6.90 (dd, 1H), 7.18 (t, 1H), 7.37-7.52 (m, 7H), 7.76-7.88 (m, 5H), 8.05 (dd, 1H), 8.14 (d, 1H), 8.21 (br s, 1H), 8.48 (br s, 1H).

LCMS Rt=3.22 minutes MS m/z 747 [M+H]⁺

### Preparation 15

### tert-Butyl 4-{2-[{[6′-(2-cyano-4-{[(2,4-dimethoxybenzyl)(1,2,4-thiadiazol-5-yl)amino]sulfonyl}phenoxy)-1,1′:3′,1″-terphenyl-3-yl]methyl}(trifluoroacetyl)amino]ethyl}piperidine-1-carboxylate

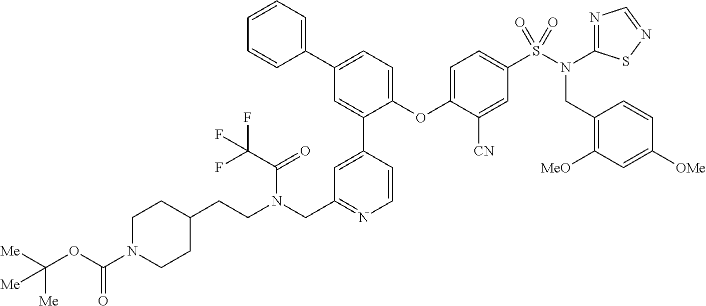 [SMILES: COc1ccc(CN(c2ncns2)S(=O)(=O)c2ccc(Oc3ccc(-c4ccccc4)cc3-c3ccnc(CN(CCC4CCN(C(=O)OC(C)(C)C)CC4)C(=O)C(F)(F)F)c3)c(C#N)c2)c(OC)c1]

Trifluoroacetic anhydride (0.40 mL, 2.88 mmol) was added to a mixture of tert-butyl 4-[2-({[6′-(2-cyano-4-{[(2,4-dimethoxybenzyl) (1,2,4-thiadiazol-5-yl)amino]sulfonyl}phenoxy)-1,1′:3′,1″-terphenyl-3-yl]methyl}amino)ethyl]piperidine-1-carboxylate (Preparation 16, 1275 mg, 1.42 mmol) and pyridine (0.46 mL, 5.69 mmol) in dichloromethane (90 mL) which was stirred for 18 hours at room temperature under nitrogen. The reaction was concentrated in vacuo to and the residue was purified using silica gel column chromatography eluting with 30% ethyl acetate in heptanes to afford the title compound as a colourless foam (1149 mg, 81%).

¹H NMR (400 MHz, CDCl₃): δ ppm 1.01-1.17 (m, 2H), 1.31-1.41 (m, 1H), 1.43-1.50 (m, 10H), 1.55-1.63 (m, 3H), 2.59-2.69 (m, 2H), 3.27-3.36 (m, 2H), 3.51 (s, 3H), 3.81 (s, 3H), 4.04 (br s, 2H), 4.64 (s, 2H), 5.25 (s, 2H), 6.16 (dd, 1H), 6.35 (dd, 1H), 6.58-6.63 (m, 1H), 7.07 (d, 1H), 7.12-7.18 (m, 1H), 7.22-7.25 (m, 1H), 7.32-7.46 (m, 4H), 7.46-7.52 (m, 2H), 7.60-7.69 (m, 6H), 8.18 (s, 1H).

LCMS Rt=4.40 minutes MS m/z 997 [M+H]⁺

### Preparation 16

### tert-Butyl 4-[2-({[6′-(2-cyano-4-{[(2,4-dimethoxybenzyl)(1,2,4-thiadiazol-5-yl)amino]sulfonyl}phenoxy)-1,1′:3′,1″-terphenyl-3-yl]methyl}amino)ethyl]piperidine-1-carboxylate

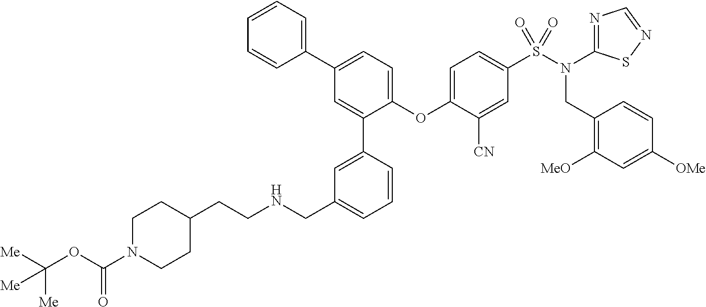 [SMILES: COc1ccc(CN(c2ncns2)S(=O)(=O)c2ccc(Oc3ccc(-c4ccccc4)cc3-c3cccc(CNCCC4CCN(C(=O)OC(C)(C)C)CC4)c3)c(C#N)c2)c(OC)c1]

Tert-butyl 4-(2-aminoethyl)piperidine-1-carboxylate (407 mg, 1.78 mmol) was added to a solution of 3-cyano-N-(2,4-dimethoxybenzyl)-4-[(3″-formyl-1,1′:3′,1″-terphenyl-4′-yl)oxy]-N-1,2,4-thiadiazol-5-ylbenzenesulfonamide (Preparation 17, 1147 mg, 1.67 mmol) in dichloromethane (19 mL) and acetic acid (0.1 mL). The mixture was stirred at room temperature under nitrogen for 45 minutes, then sodium triacetoxyborohydride (409 mg, 1.93 mmol) was added and the reaction was stirred for 18 hours under nitrogen at room temperature. The reaction was diluted with dichloromethane (100 mL) and washed with water (3×10 mL). The aqueous layers were extracted with dichloromethane (3×10 mL). The combined organic layers were dried over sodium sulfate, filtered and concentrated in vacuo to afford a colourless foam that was purified using silica gel column chromatography eluting with 1-20% MeOH in EtOAc to afford the title compound as a colourless foam (1275 mg, 85%).

¹H NMR (400 MHz, CDCl₃): δ ppm 1.04-1.15 (m, 2H), 1.44-1.49 (m, 13H), 1.63 (br d, 2H), 2.61-2.70 (m, 4H), 3.47 (s, 3H), 3.81 (s, 3H), 3.82 (s, 2H), 4.05 (br s, 2H), 5.24 (s, 2H), 6.12 (d, 1H), 6.35 (dd, 1H), 6.57 (d, 1H), 7.07 (d, 1H), 7.23 (d, 1H), 7.27-7.28 (m, 1H), 7.31 (t, 1H), 7.37-7.43 (m, 2H), 7.46-7.53 (m, 3H), 7.58 (d, 1H), 7.61-7.66 (m, 4H), 7.72 (d, 1H), 8.17 (s, 1H).

LCMS Rt=3.90 minutes MS m/z 901 [M+H]⁺

### Preparation 17

### 3-Cyano-N-(2,4-dimethoxybenzyl)-4-[(3″-formyl-1,1′:3′,1″-terphenyl-4′-yl)oxy]-N-1,2,4-thiadiazol-5-ylbenzenesulfonamide

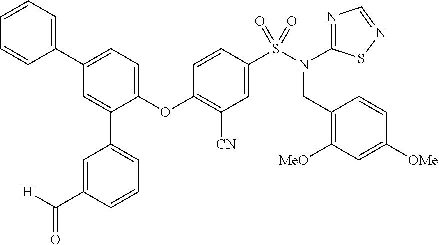 [SMILES: COc1ccc(CN(c2ncns2)S(=O)(=O)c2ccc(Oc3ccc(-c4ccccc4)cc3-c3cccc(C=O)c3)c(C#N)c2)c(OC)c1]

A solution of 6′-hydroxy-1,1′:3′,1″-terphenyl-3-carbaldehyde (Preparation 38, 668 mg, 2.43 mmol), 3-cyano-N-(2,4-dimethoxybenzyl)-4-fluoro-N-(1,2,4-thiadiazol-5-yl)benzenesulfonamide (WO2010079443, 1004 mg, 2.31 mmol) and potassium carbonate (954 mg, 6.90 mmol) in dimethylsulfoxide (10 mL) was stirred for 18 hours at room temperature under nitrogen. The reaction was diluted with water (100 mL) and extracted with ethyl acetate (3×60 mL). The combined organic layers were washed with brine (3×80 mL), dried over sodium sulfate, filtered and concentrated in vacuo to afford a yellow foam that was purified using silica gel column chromatography eluting with 30% EtOAc in heptanes to afford the title compound as a colourless foam (1147 mg, 72%).

¹H NMR (400 MHz, CDCl₃): δ ppm 3.44 (s, 3H), 3.81 (s, 3H), 5.24 (s, 2H), 6.02 (d, 1H), 6.34 (dd, 1H), 6.60 (d, 1H), 7.07 (d, 1H), 7.24-7.26 (m, 1H), 7.40-7.44 (m, 1H), 7.47-7.52 (m, 2H), 7.56-7.60 (m, 2H), 7.61-7.68 (m, 3H), 7.70 (dd, 1H), 7.76 (d, 1H), 7.85-7.87 (m, 2H), 8.06-8.07 (m, 1H), 8.17 (s, 1H), 10.05 (s, 1H).

LCMS Rt=4.03 minutes MS m/z No mass ion observed

### Preparation 18

### 3-cyano-N-(2,4-dimethoxybenzyl)-4-((3-(2-(hydroxymethyl)pyridin-4-yl)-3′-(trifluoromethyl)-[1,1′-biphenyl]-4-yl)oxy)-N-(1,2,4-thiadiazol-5-yl)benzenesulfonamide

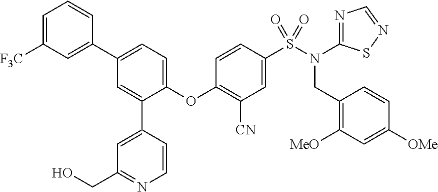 [SMILES: COc1ccc(CN(c2ncns2)S(=O)(=O)c2ccc(Oc3ccc(-c4cccc(C(F)(F)F)c4)cc3-c3ccnc(CO)c3)c(C#N)c2)c(OC)c1]

The title compound was prepared according to the method described for Preparation 8 using 3-cyano-N-(2,4-dimethoxybenzyl)-4-fluoro-N-(1,2,4-thiadiazol-5-yl)benzenesulfonamide (WO2010079443) and 3-(2-(hydroxymethyl)pyridin-4-yl)-3′-(trifluoromethyl)-[1,1′-biphenyl]-4-ol (Preparation 37). The residue was purified using silica gel column chromatography eluting with 0-100% EtOAc in heptanes.

¹H NMR (400 MHz, DMSO-d₆): δ ppm 3.56 (s, 3H), 3.73 (s, 3H), 4.55 (d, 2H), 5.18 (s, 2H), 5.34 (t, 1H), 6.35 (m, 1H), 6.44-6.45 (m, 1H), 6.98-7.02 (m, 2H), 7.44 (m, 1H), 7.52 (m, 1H), 7.61 (m, 1H), 7.74-7.78 (m, 2H), 7.96-8.02 (m, 2H), 8.12-8.14 (m, 2H), 8.41 (m, 1H), 8.51 (m, 1H).

MS m/z 760 [M+H]⁺

### Preparation 19

### tert-Butyl 4-{2-[({4-[4-{2-cyano-4-[(1,2,4-thiadiazol-5-ylamino)sulfonyl]phenoxy}-3′-(trifluoromethyl)biphenyl-3-yl]pyridin-2-yl}methyl)amino]ethyl}piperidine-1-carboxylate

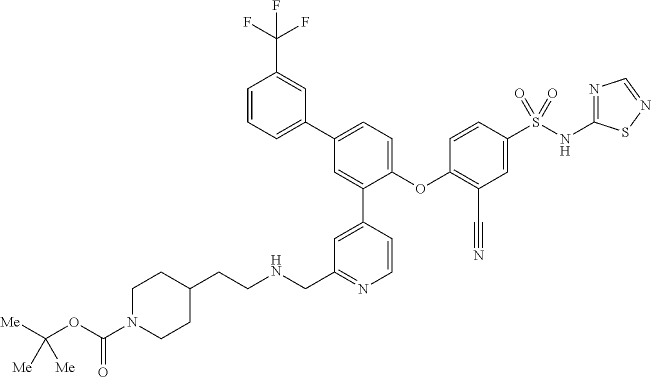 [SMILES: CC(C)(C)OC(=O)N1CCC(CCNCc2cc(-c3cc(-c4cccc(C(F)(F)F)c4)ccc3Oc3ccc(S(=O)(=O)Nc4ncns4)cc3C#N)ccn2)CC1]

To a suspension of 4-({3-[2-(aminomethyl)pyridin-4-yl]-3′-(trifluoromethyl)biphenyl-4-yl}oxy)-3-cyano-N-1,2,4-thiadiazol-5-ylbenzenesulfonamide bis-formate salt (Example 26, 50 mg, 0.071 mmol) in methanol (2 mL) was added triethylamine (20 μL, 0.143 mmol) followed by tert-butyl 4-(2-oxoethyl)piperidine-1-carboxylate (16 mg, 0.071 mmol). The reaction was stirred at room temperature for 18 hours and sodium borohydride (16 mg, 0.43 mmol) was added. After 30 minutes at room temperature, the mixture was quenched by the addition of water (5.0 mL). The aqueous phase was extracted with ethyl acetate (3×5 mL) and the combined organic layers were washed with brine (10 mL), dried over anhydrous magnesium sulfate, filtered and concentrated in vacuo. The residue was purified by reverse phase column chromatography eluting with 5-95% acetonitrile in water with 0.1% formic acid to afford the title compound as a white solid (35 mg, 62%).

¹H NMR (400 MHz, MeOD-d₄): δ ppm 1.05 (m, 2H), 1.40 (s, 9H), 1.40-1.80 (m, 5H), 2.70 (m, 2H), 3.05 (m, 2H), 4.00 (d, 2H), 4.40 (s, 2H), 6.80 (d, 1H), 7.40 (d, 1H), 7.50 (d, 1H), 7.60 (m, 2H), 7.80 (m, 3H), 7.95 (m, 3H), 8.05 (s, 1H), 8.50 (d, 2H).

¹⁹F NMR (400 MHz, MeOD-d₄): δ ppm −64 (s, 3F).

### Preparation 20

### tert-Butyl 4-(2-{[(6′-{2-cyano-4-[(1,2,4-thiadiazol-5-ylamino)sulfonyl]phenoxy}-1,1′:3′,1″-terphenyl-3-yl)carbonyl]amino}ethyl)piperazine-1-carboxylate

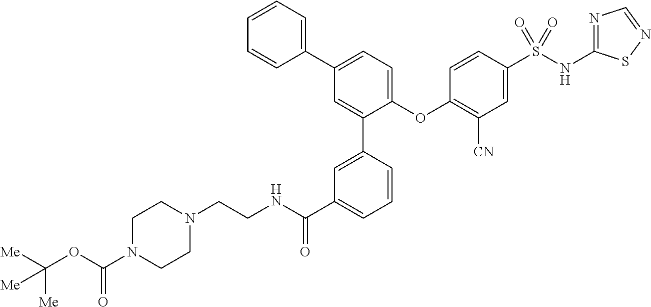 [SMILES: CC(C)(C)OC(=O)N1CCN(CCNC(=O)c2cccc(-c3cc(-c4ccccc4)ccc3Oc3ccc(S(=O)(=O)Nc4ncns4)cc3C#N)c2)CC1]

To a solution of 6′-{2-cyano-4-[(1,2,4-thiadiazol-5-ylamino)sulfonyl]phenoxy}-1,1′:3′,1″-terphenyl-3-carboxylic acid (Preparation 21, 100 mg, 0.18 mmol) in dimethylformamide (2.0 mL) was added 1,1′-carbonylbis(1H-imidazole) (38 mg, 0.23 mmol) and N-ethyl-N-isopropylpropan-2-amine (35 mg, 0.27 mmol). The mixture was stirred at room temperature for 30 minutes, then tert-butyl 4-(2-aminoethyl)piperazine-1-carboxylate (41.3 mg, 0.18 mmol) was added. The resulting reaction was stirred at room temperature for 3 days. The mixture was concentrated in vacuo to provide the title compound as an orange gum (204 mg, >100%). This material was used in the next step without further purification.

LCMS Rt=2.40 minutes MS m/z 764 [M−H]⁻

### Preparation 21

### 6′-{2-Cyano-4-[(1,2,4-thiadiazol-5-ylamino)sulfonyl]phenoxy}-1,1′:3′,1″-terphenyl-3-carboxylic acid

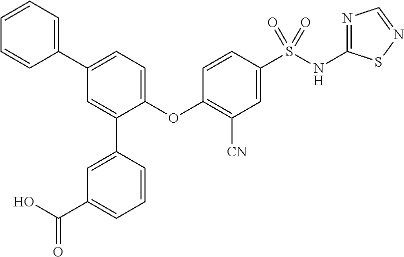 [SMILES: N#Cc1cc(S(=O)(=O)Nc2ncns2)ccc1Oc1ccc(-c2ccccc2)cc1-c1cccc(C(=O)O)c1]

A solution of 3-cyano-4-[(3-iodobiphenyl-4-yl)oxy]-N-1,2,4-thiadiazol-5-ylbenzenesulfonamide (Preparation 22, 450 mg, 0.80 mmol), 3-(dihydroxyboryl)benzoic acid (200 mg, 1.20 mmol), and sodium carbonate (340 mg, 3.21 mmol) in DME (2.0 mL) and water (1.5 mL) was degassed with nitrogen then bis(triphenylphosphine) palladium(II)chloride (56 mg, 0.08 mmol) was added and the reaction mixture was heated for 15 minutes at 150° C. under microwave irradiation. The mixture was cooled, diluted with water (50 mL) and ethyl acetate (200 mL). The aqueous was acidified to pH=3 using a 4N aqueous solution of hydrochloric acid, then extracted with ethyl acetate (3×20 mL). The organic layers were combined, dried over magnesium sulfate, filtered and concentrated in vacuo to yield the crude product as an orange solid. The crude material was purified by silica gel column chromatography eluting with DCM to DCM:MeOH:formic acid (100:10:0.1) to afford the title compound as an orange solid (200 mg, 45%).

¹H NMR (400 MHz, DMSO-d₆): δ ppm 6.94 (d, 1H), 7.37-7.65 (m, 6H), 7.73-8.10 (m, 9H).

LCMS Rt=2.05 minutes MS m/z 503 [M−H]⁻

### Preparation 22

### 3-Cyano-4-[(3-iodobiphenyl-4-yl)oxy]-N-1,2,4-thiadiazol-5-ylbenzenesulfonamide

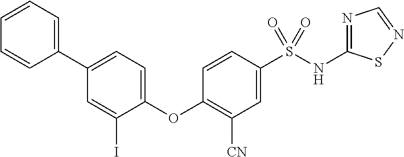 [SMILES: N#Cc1cc(S(=O)(=O)Nc2ncns2)ccc1Oc1ccc(-c2ccccc2)cc1I]

A solution of 3-Iodobiphenyl-4-ol (Preparation 78, 500 mg, 1.69 mmol), 3-cyano-4-fluoro-N-1,2,4-thiadiazol-5-ylbenzenesulfonamide (WO2010079443, 480 mg, 1.69 mmol) and potassium carbonate (700 mg, 5.07 mmol) in dimethylformamide (2 mL) was stirred for 18 hours at 80° C. under nitrogen. The reaction was cooled, diluted with water (10 mL), neutralised using 4N HCl, then extracted with ethyl acetate (3×20 mL). The combined organic layers were dried over magnesium sulfate, filtered and concentrated in vacuo to afford a yellow solid. The crude material was recystallised from DCM to afford the title compound as a white solid (450 mg, 48%).

¹H NMR (400 MHz, DMSO-d₆): δ ppm 6.88 (d, 1H), 7.35-7.58 (m, 4H), 7.70 (d, 2H), 7.79 (d, 1H), 8.01 (d, 1H), 8.22 (s, 1H), 8.50 (s, 1H), 8.47 (s, 1H).

LCMS Rt=1.60 minutes MS m/z 561 [M+H]⁺

### Preparation 23

### 4-{[3″-({[2-(1-Acetylpiperidin-4-yl)ethyl]amino}methyl)-1,1′:3′,1″-terphenyl-4′-yl]oxy}-3-cyano-N-(2,4-dimethoxybenzyl)-N-1,2,4-thiadiazol-5-ylbenzenesulfonamide

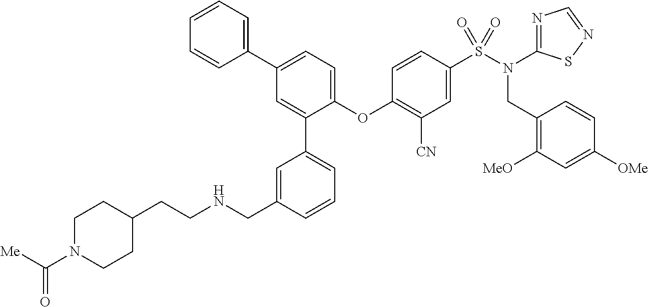 [SMILES: COc1ccc(CN(c2ncns2)S(=O)(=O)c2ccc(Oc3ccc(-c4ccccc4)cc3-c3cccc(CNCCC4CCN(C(C)=O)CC4)c3)c(C#N)c2)c(OC)c1]

To a solution of 2-(1-acetylpiperidin-4-yl)ethanamine (Preparation 79, 740 mg, 4.35 mmol) and 3-cyano-N-(2,4-dimethoxybenzyl)-4-[(3″-formyl-1,1′:3′,1″-terphenyl-4′-yl)oxy]-N-1,2,4-thiadiazol-5-ylbenzenesulfonamide (Preparation 17, 1 g, 1.45 mmol) in dioxane (20 mL) was added acetic acid (10 drops). The reaction mixture was stirred for 3 hours at 70° C. then allowed to cool to room temperature. Sodium borohydride (165 mg, 4.35 mmol) was added and the reaction was stirred for 3 hours at room temperature. Water (50 mL) was added and the solution was concentrated in vacuo. The aqueous suspension was extracted with ethyl acetate (2×200 mL). The organic layers were combined, dried over sodium sulfate, filtered and concentrated in vacuo to afford the title compound as a viscous oil (2 g, crude material).

LCMS Rt=2.54 minutes MS m/z 843 [M+H]⁺

### Preparation 24

### tert-Butyl ({4-[4-(2-cyano-4-{[(2,4-dimethoxybenzyl)(1,2,4-thiadiazol-5-yl)amino]sulfonyl}phenoxy)-3′-(trifluoromethyl) biphenyl-3-yl]pyridin-2-yl}methyl)carbamate

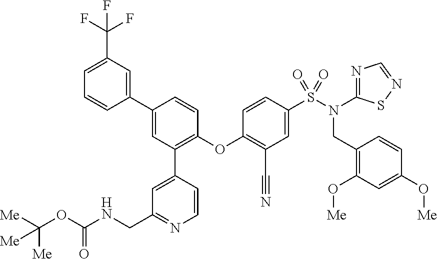 [SMILES: COc1ccc(CN(c2ncns2)S(=O)(=O)c2ccc(Oc3ccc(-c4cccc(C(F)(F)F)c4)cc3-c3ccnc(CNC(=O)OC(C)(C)C)c3)c(C#N)c2)c(OC)c1]

tert-Butyl ({4-[4-hydroxy-3′-(trifluoromethyl) biphenyl-3-yl]pyridin-2-yl}methyl)carbamate (Preparation 41, 850 mg, 1.91 mmol) was dissolved in dimethyl sulfoxide (10 mL) and potassium carbonate (529 mg, 3.83 mmol) followed by 3-cyano-N-(2,4-dimethoxybenzyl)-4-fluoro-N-(1,2,4-thiadiazol-5-yl)benzenesulfonamide (WO2010079443, 831 mg, 1.91 mmol) were added. The reaction mixture was stirred at room temperature for 18 hours. The reaction was partitioned between ethyl acetate (100 mL) and water (50 mL). The organic layer was dried over magnesium sulfate and evaporated in vacuo. The residue was purified by silica gel column chromatography eluting with 7:3 ethyl acetate:heptane followed by purification by reverse phase column chromatography eluting with acetonitrile/water with 0.1% formic acid to afford the title compound as a foam (683 mg, 41%).

¹H NMR (400 MHz, CDCl₃): δ ppm 1.44 (s, 9H), 3.42 (s, 3H), 3.83 (s, 3H), 4.51 (d, 2H), 5.27 (s, 2H), 5.66 (br s, 1H), 6.07 (s, 1H), 6.38-6.34 (dd, 1H), 6.61 (d, 1H), 7.10 (d, 1H), 7.27 (d, 1H), 7.39-7.37 (dd, 1H), 7.48 (s, 1H), 7.74-7.59 (m, 6H), 7.80 (d, 1H), 7.84 (s, 1H), 8.18 (s, 1H), 8.58 (d, 1H).

¹⁹F NMR (400 MHz, CDCl₃): δ ppm −62.7 (s, 3F).

LCMS Rt=4.07 minutes MS m/z 859 [M+H]⁺

### Preparation 25

### tert-Butyl 4-(2-{[(benzyloxy)carbonyl]({4-[4-hydroxy-3′-(trifluoromethyl)biphenyl-3-yl]pyridin-2-yl}methyl)amino}ethyl)piperidine-1-carboxylate

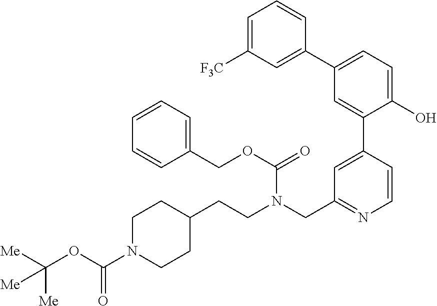 [SMILES: CC(C)(C)OC(=O)N1CCC(CCN(Cc2cc(-c3cc(-c4cccc(C(F)(F)F)c4)ccc3O)ccn2)C(=O)OCc2ccccc2)CC1]

tert-Butyl 4-(2-{[(benzyloxy)carbonyl][(4-bromopyridin-2-yl)methyl]amino}ethyl)piperidine-1-carboxylate (Preparation 62, 2.50 g, 4.70 mmol), 3-(4,4,5,5-tetramethyl-1,3,2-dioxaborolan-2-yl)-3′-(trifluoromethyl)biphenyl-4-ol (Preparation 68, 1.71 g, 4.70 mmol) and sodium carbonate (1.99 g, 18.8 mmol) in a mixture of dioxane (40 mL) and water (8 mL) were degassed. Tetrakis(triphenylphosphine)palladium(0) (0.27 g, 0.24 mmol) was added and the reaction mixture was further degassed and heated at 80° C. for 1 hour. The reaction was cooled to room temperature, diluted with ethyl acetate (50 mL), washed with water (30 mL), and the organic layer was dried over magnesium sulfate and filtrate was evaporated in vacuo. The residue was purified by silica gel column chromatography eluting with 1:1 ethyl acetate:cyclohexane to give the title compound as yellow foam (2.65 g, 82%).

¹H NMR (400 MHz, CDCl₃): δ ppm 0.98-1.13 (m, 2H), 1.39-1.63 (m, 14H), 2.57 (br s, 2H), 3.42 (br s, 2H), 3.99 (br s, 2H), 4.66 (d, 2H), 5.16 (d, 2H), 6.23 (s, 1H), 7.04 (d, 1H), 7.16-7.34 (m, 4H), 7.41 (br s, 2H), 7.48-7.60 (m, 4H), 7.71 (br s, 1H), 7.78 (s, 1H), 8.61 (d, 1H).

¹⁹F NMR (400 MHz, CDCl₃): δ ppm −62.6 (s, 3F).

LCMS Rt=2.98 minutes MS m/z 690 [M+H]⁺

### Preparation 26

### tert-Butyl 4-(2-{({4-[4-hydroxy-3′-(trifluoromethyl)biphenyl-3-yl]pyridin-2-yl}methyl)[(2,2,2-trichloroethoxy)carbonyl]amino}ethyl)piperidine-1-carboxylate

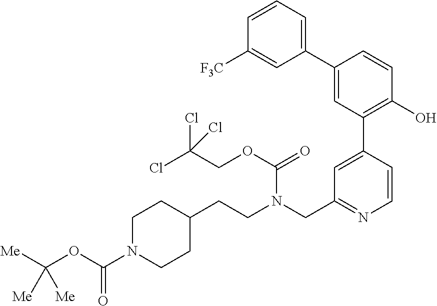 [SMILES: CC(C)(C)OC(=O)N1CCC(CCN(Cc2cc(-c3cc(-c4cccc(C(F)(F)F)c4)ccc3O)ccn2)C(=O)OCC(Cl)(Cl)Cl)CC1]

tert-Butyl 4-(2-{[(4-bromopyridin-2-yl)methyl][(2,2,2-trichloroethoxy)carbonyl]amino}ethyl)piperidine-1-carboxylate (Preparation 60, 5 g, 8.72 mmol), 3-(4,4,5,5-tetramethyl-1,3,2-dioxaborolan-2-yl)-3′-(trifluoromethyl)biphenyl-4-ol (Preparation 68, 3.52 g, 9.67 mmol) and sodium carbonate (4.09 g, 38.7 mmol) were combined and dissolved in a mixture of 1,4 dioxane/water (4/1 60 mL). The reaction was degassed for 20 minutes and tetrakis(triphenylphosphine)palladium(0) (560 mg, 0.48 mmol) was added in one portion. The reaction was heated at 100° C. for 2 hours, cooled to room temperature and partitioned between ethyl acetate (50 ml) and water (50 mL). The organic layer was washed with brine (50 mL), dried over anhydrous magnesium sulfate and concentrated in vacuo. The residue was purified by silica gel column chromatography eluting with 20-60% EtOAc in cyclohexanes to afford the title compound as a yellow foam (3.50 g, 55%).

¹H NMR (400 MHz, CDCl₃): δ ppm 1.05 (m, 2H), 1.20 (s, 9H), 1.60 (m, 3H), 1.80 (m, 2H), 2.05 (m, 2H), 2.60 (m, 2H), 3.40 (m, 2H), 4.00 (m, 2H), 4.70 (m, 2H), 7.05 (d, 1H), 7.55 (m, 3H), 7.60 (m, 3H), 7.70 (d, 1H), 7.80 (s, 1H), 8.60 (d, 1H).

¹⁹F NMR (400 MHz, CDCl₃): δ ppm −64.0. (s, 3F).

LCMS Rt=3.31 minutes MS m/z 730 [M+H]⁺

### Preparation 27

### (4-(4-hydroxy-3′-(trifluoromethyl)-[1,1′-biphenyl]-3-yl)pyridin-2-yl)methyl (2,5,8,11-tetraoxatridecan-13-yl)carbamate

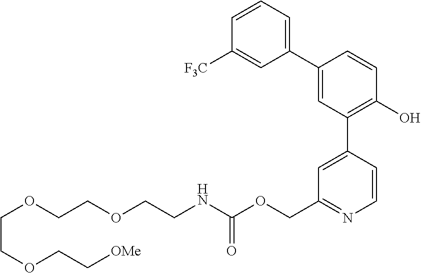 [SMILES: COCCOCCOCCOCCNC(=O)OCc1cc(-c2cc(-c3cccc(C(F)(F)F)c3)ccc2O)ccn1]

To a solution of 3-(2-(hydroxymethyl)pyridin-4-yl)-3′-(trifluoromethyl)-[1,1′-biphenyl]-4-ol (Preparation 36, 60 mg, 0.175 mmol) in DMF (1 mL) was added sodium hydride (22 mg, 0.525 mmol) at 0° C. and the reaction was stirred at room temperature for 40 minutes. 4-nitrophenyl (2,5,8,11-tetraoxatridecan-13-yl)carbamate (Preparation 67, 65 mg, 0.18 mmol) was added and the reaction was stirred at room temperature for 2 hours followed by 45° C. for 4 hours. The reaction was acidified to pH=5-6 with 1N HCl (aq) and extracted into EtOAc. The organic layer was collected and purified using silica gel column chromatography eluting with 0-10% MeOH in DCM to afford the title compound (7 mg, 7%).

¹H NMR (400 MHz, CDCl₃): δ ppm 3.35 (s, 3H), 3.40-3.70 (m, 16H), 5.30 (s, 2H), 7.10 (m, 1H), 7.25 (s, 1H), 7.40 (m, 1H), 7.45-7.60 (m, 4H), 7.70-7.80 (m, 3H), 8.60 (m, 1H).

MS m/z 579 [M+H]⁺

### Preparation 28

### tert-butyl (2,5,8,11,14,17,20,23,26,29,32,35-dodecaoxaheptatriacontan-37-yl)((4-(4-hydroxy-3′-(trifluoromethyl)-[1,1′-biphenyl]-3-yl)pyridin-2-yl)methyl)carbamate

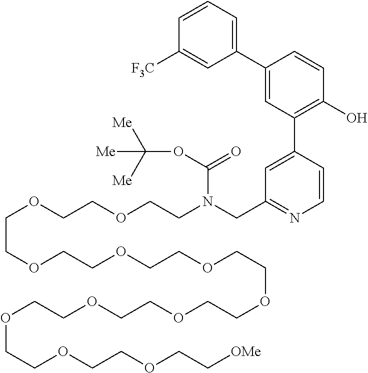 [SMILES: COCCOCCOCCOCCOCCOCCOCCOCCOCCOCCOCCOCCN(Cc1cc(-c2cc(-c3cccc(C(F)(F)F)c3)ccc2O)ccn1)C(=O)OC(C)(C)C]

To a solution of tert-butyl (2,5,8,11,14,17,20,23,26,29,32,35-dodecaoxaheptatriacontan-37-yl) ((4-(4-methoxy-3′-(trifluoromethyl)-[1,1′-biphenyl]-3-yl)pyridin-2-yl)methyl)carbamate (Preparation 44, 200 mg, 0.20 mmol) in DMSO (2.3 mL) was added 1-decane-thiol (76 uL, 0.3 mmol) followed by solid sodium hydroxide (24 mg, 0.66 mmol) and the reaction was heated to 120° C. for 2 hours. Further 1-decane-thiol (76 uL, 0.3 mmol) was added and the reaction continued at 120° C. for 2 hours. The reaction was cooled, quenched with water and acidified to pH=5-6 with 1N HCl (aq). The solution was extracted into EtOAc three times. The organic layers were collected, washed with brine, dried over sodium sulphate and concentrated in vacuo. The residue was purified using silica gel column chromatography eluting with 0-15% MeOH in DCM to afford the title compound (108 mg, 93%).

MS m/z 987 [M+H]⁺

### Preparation 29

### tert-butyl ((4-(4-hydroxy-3′-(trifluoromethyl)-[1,1′-biphenyl]-3-yl)pyridin-2-yl)methyl)(2,5,8,11-tetraoxatridecan-13-yl)carbamate

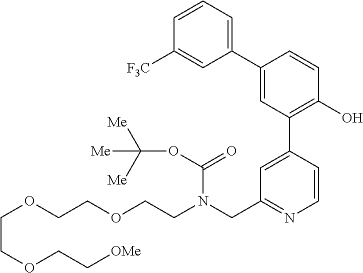 [SMILES: COCCOCCOCCOCCN(Cc1cc(-c2cc(-c3cccc(C(F)(F)F)c3)ccc2O)ccn1)C(=O)OC(C)(C)C]

To a solution of tert-butyl ((4-(4-methoxy-3′-(trifluoromethyl)-[1,1′-biphenyl]-3-yl)pyridin-2-yl)methyl)(2,5,8,11-tetraoxatridecan-13-yl)carbamate (Preparation 45, 71 mg, 0.11 mmol) in DMSO (1 mL) was added 1-decane-thiol (42 uL, 0.165 mmol) followed by solid NaOH (130 mg, 0.330 mmol) and the reaction was heated to 120° C. for 18 hours. Further 1-decane-thiol (42 uL, 0.165 mmol) followed by solid NaOH (130 mg, 0.330 mmol) were added and the reaction continued at 120° C. for 1.5 hours. The reaction was cooled, quenched with water and acidified to pH=5-6 with 1N HCl (aq). The solution was extracted into EtOAc three times. The organic layers were collected, washed with brine, dried over sodium sulphate and concentrated in vacuo. The residue was purified using silica gel column chromatography eluting with 0-15% MeOH in DCM to afford the title compound (65 mg, 93%).

MS m/z 534 [(M-Boc)+H]+

### Preparation 30

### 3-(2-(2,5,8,11,14-pentaoxapentadecyl)pyridin-4-yl)-3′-(trifluoromethyl)-[1,1′-biphenyl]-4-ol

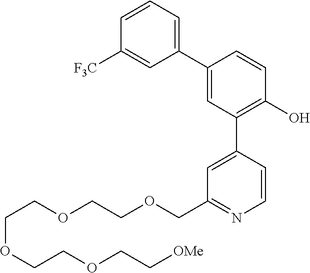 [SMILES: COCCOCCOCCOCCOCc1cc(-c2cc(-c3cccc(C(F)(F)F)c3)ccc2O)ccn1]

The title compound was prepared according to the method described for Preparation 29 using 4-(4-methoxy-3′-(trifluoromethyl)-[1,1′-biphenyl]-3-yl)-2-(2,5,8,11,14-pentaoxapentadecyl)pyridine (Preparation 52).

¹H NMR (400 MHz, CDCl₃): δ ppm 3.35 (s, 3H), 3.50-3.80 (m, 16H), 4.80 (s, 2H), 7.15 (m, 1H), 7.50-7.60 (m, 5H), 7.75 (m, 1H), 7.80 (m, 1H), 8.10 (m, 1H), 8.65 (m, 1H).

MS m/z 536 [M+H]⁺

### Preparation 31

### 2-((4-(4-hydroxy-3′-(trifluoromethyl)-[1,1′-biphenyl]-3-yl)pyridin-2-yl)methoxy)-N-(2,5,8,11-tetraoxatridecan-13-yl)acetamide

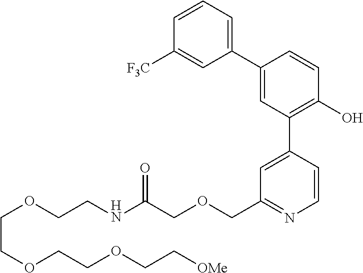 [SMILES: COCCOCCOCCOCCNC(=O)COCc1cc(-c2cc(-c3cccc(C(F)(F)F)c3)ccc2O)ccn1]

The title compound was prepared according to the method described for Preparation 29 using 2-((4-(4-methoxy-3′-(trifluoromethyl)-[1,1′-biphenyl]-3-yl)pyridin-2-yl)methoxy)-N-(2,5,8,11-tetraoxatridecan-13-yl)acetamide (Preparation 54).

¹H NMR (400 MHz, CDCl₃): δ ppm 3.35 (s, 3H), 3.50-3.65 (m, 16H), 4.20 (s, 2H), 4.80 (s, 2H), 7.18 (m, 2H), 7.40-7.80 (m, 8H), 7.95 (m, 1H), 8.60 (m, 1H).

MS m/z 593 [M+H]⁺

### Preparation 32

### 3-(2-(2,8,11,14,17-pentaoxa-5-azaoctadecyl)pyridin-4-yl)-3′-(trifluoromethyl)-[1,1′-biphenyl]-4-ol

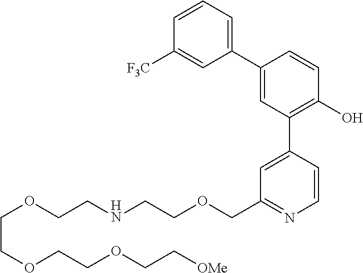 [SMILES: COCCOCCOCCOCCNCCOCc1cc(-c2cc(-c3cccc(C(F)(F)F)c3)ccc2O)ccn1]

The title compound was prepared according to the method described for Preparation 29 using N-(2-((4-(4-methoxy-3′-(trifluoromethyl)-[1,1′-biphenyl]-3-yl)pyridin-2-yl)methoxy)ethyl)-2,5,8,11-tetraoxatridecan-13-amine (Preparation 53).

MS m/z 577 [M−H]⁻

### Preparation 33

### 1-((4-(4-hydroxy-3′-(trifluoromethyl)-[1,1′-biphenyl]-3-yl)pyridin-2-yl)methyl)-3-(2,5,8,11-tetraoxatridecan-13-yl)urea

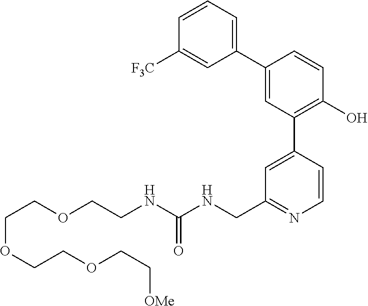 [SMILES: COCCOCCOCCOCCNC(=O)NCc1cc(-c2cc(-c3cccc(C(F)(F)F)c3)ccc2O)ccn1]

The title compound was prepared according to the method described for Preparation 29 using 1-((4-(4-methoxy-3′-(trifluoromethyl)-[1,1′-biphenyl]-3-yl)pyridin-2-yl)methyl)-3-(2,5,8,11-tetraoxatridecan-13-yl)urea (Preparation 48).

¹H NMR (400 MHz, CDCl₃): δ ppm 3.30 (s, 3H), 3.40-3.70 (m, 16H), 4.56-4.61 (m, 2H), 5.90 (br s, 1H), 6.20 (br s, 1H), 7.06-7.10 (m, 1H), 7.32-7.36 (m, 1H), 7.39-7.45 (m, 2H), 7.45-7.52 (m, 1H), 7.52-7.57 (m, 1H), 7.64-7.68 (m, 1H), 7.70-7.77 (m, 2H), 8.53-8.58 (m, 1H).

MS m/z 578 [M+H]⁺

### Preparation 34

### 2-(2-(2-methoxyethoxy)ethoxy)ethyl ((4-(4-hydroxy-3′-(trifluoromethyl)-[1,1′-biphenyl]-3-yl)pyridin-2-yl)methyl)carbamate

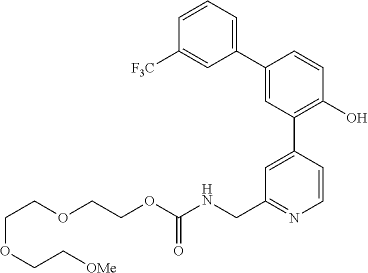 [SMILES: COCCOCCOCCOC(=O)NCc1cc(-c2cc(-c3cccc(C(F)(F)F)c3)ccc2O)ccn1]

To a solution of 3-(2-(aminomethyl)pyridin-4-yl)-3′-(trifluoromethyl)-[1,1′-biphenyl]-4-ol (Preparation 35, 60 mg, 0.17 mmol) and 2,5-dioxopyrrolidin-1-yl (2-(2-(2-methoxyethoxy)ethoxy)ethyl) carbonate (53 mg, 0.174 mmol) in isopropanol (1.5 mL) was added DIPEA (0.1 mL) and the reaction was heated at 52° C. for 18 hours. The reaction was concentrated in vacuo and partitioned between EtOAc and water. The organic layer was collected, dried over sodium sulphate and concentrated in vacuo. The residue was purified using silica gel column chromatography eluting with 10% MeOH in DCM to afford the title compound (26 mg, 28%).

¹H NMR (500 MHz, MeOH-d₄): δ ppm 3.35 (m, 5H), 3.40-3.60 (m, 6H), 3.70 (t, 2H), 4.20 (m, 2H), 4.45 (m, 2H), 7.05 (m, 1H), 7.55-7.70 (m, 5H), 7.90 (m, 2H), 8.50 (m, 1H).

### Preparation 35

### 3-(2-(aminomethyl)pyridin-4-yl)-3′-(trifluoromethyl)-[1,1′-biphenyl]-4-ol

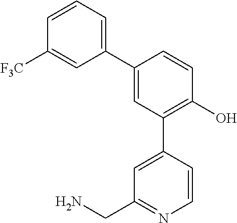 [SMILES: NCc1cc(-c2cc(-c3cccc(C(F)(F)F)c3)ccc2O)ccn1]

To a solution of (4-(4-methoxy-3′-(trifluoromethyl)-[1,1′-biphenyl]-3-yl)pyridin-2-yl)methanamine (Preparation 49, 60 mg, 0.17 mmol) in DMSO (0.5 mL) was added 1-decane thiol (106 mg, 0.5 mmol) followed by solid sodium hydroxide (13 mg, 0.33 mmol). The reaction was heated to 120° C. for 2 hours then cooled to 0° C. and quenched by the addition of 1N HCl (aq). The reaction mixture was extracted with EtOAc, the organic layer collected, washed with brine, dried over sodium sulphate and concentrated in vacuo. The residue was purified using 10% MeOH in DCM with 3% TEA to afford the title compound (58 mg, 100%).

¹H NMR (500 MHz, MeOH-d₄): δ ppm 4.33 (s, 2H), 7.06-7.11 (m, 1H), 7.61 (d, 3H), 7.66 (s, 1H), 7.72-7.76 (m, 1H), 7.77-7.80 (m, 1H), 7.84-7.91 (m, 2H), 8.63-8.66 (m, 1H).

### Preparation 36

### 3-(2-(hydroxymethyl)pyridin-4-yl)-3′-(trifluoromethyl)-[1,1′-biphenyl]-4-ol

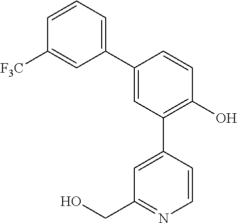 [SMILES: OCc1cc(-c2cc(-c3cccc(C(F)(F)F)c3)ccc2O)ccn1]

The title compound was prepared according to the method described for Preparation using (4-(4-methoxy-3′-(trifluoromethyl)-[1,1′-biphenyl]-3-yl)pyridin-2-yl)methanol (Preparation 57) and taken on directly to the next step.

### Preparation 37

### 3-(2-(hydroxymethyl)pyridin-4-yl)-3′-(trifluoromethyl)-[1,1′-biphenyl]-4-ol

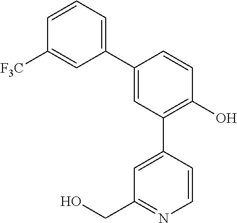 [SMILES: OCc1cc(-c2cc(-c3cccc(C(F)(F)F)c3)ccc2O)ccn1]

The title compound was prepared according to the method described for Preparation 58 using 4-chloro-2-(2-(hydroxymethyl)pyridin-4-yl)phenol (Preparation 42) and 3-trifluoromethylphenyl boronic acid at 120° C. for 2 hours under microwave irradiation. The residue was purified using silica gel column chromatography eluting with 0-10% MeOH in DCM. The intermediate was taken directly on to the next step.

### Preparation 38

### 6′-Hydroxy-1,1′:3′,1″-terphenyl-3-carbaldehyde

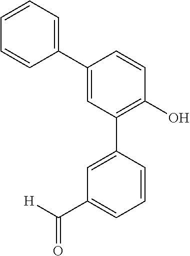 [SMILES: O=Cc1cccc(-c2cc(-c3ccccc3)ccc2O)c1]

A solution of 3-iodobiphenyl-4-ol (Preparation 78, 12.8 g, 43.2 mmol), 3-formylphenylboronic acid (12.9 g, 86.0 mmol), and cesium carbonate (35.2 g, 108.0 mmol) in dioxane (260 mL) and water (70 mL) was degassed for 1 hour with nitrogen then [1,1′-bis(diphenylphosphino)ferrocene]dichloro palladium (11) (3.2 g, 4.37 mmol) was added and the reaction mixture was heated for 18 hours at 90° C. The solution was filtered through a pad of celite which was washed with methanol (250 mL) and ethyl acetate (250 mL). The filtrate was concentrated in vacuo, partitioned between water (200 mL) and ethyl acetate (200 mL). The aqueous solution was acidified to pH=1-2 using an aqueous solution of hydrochloric acid extracted with ethyl acetate (3×200 mL). The organic layers were combined, dried over sodium sulfate, filtered and concentrated in vacuo. The crude material was purified twice by silica gel column chromatography eluting with 20% ethyl acetate in cyclohexane to afford the title compound as a light brown solid (6.28 g, 53%).

¹H NMR (400 MHz, CDCl₃): δ ppm 5.19 (s, 1H), 7.05 (m, 1H), 7.33 (m, 1H), 7.43 (m, 2H), 7.53 (m, 2H), 7.58 (m, 2H), 7.67 (m, 1H), 7.85 (m, 1H), 7.93 (m, 1H), 8.09 (m, 1H), 10.09 (s, 1H).

LCMS Rt=3.31 minutes MS m/z 273 [M−H]⁻

### Preparation 39

### 3-Bromo-3′-(trifluoromethyl)biphenyl-4-ol

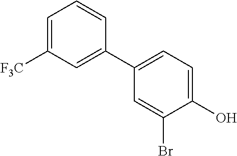 [SMILES: Oc1ccc(-c2cccc(C(F)(F)F)c2)cc1Br]

To a solution of 3′-(trifluoromethyl)biphenyl-4-ol (Preparation 40, 29 g, 122 mmol) in a mixture of dichloromethane/acetic acid (1/1, 400 mL) cooled at 0° C. in an ice bath was added sulphuric acid (1 mL) followed by N-bromo-succinimide (19.6 g, 110 mmol) over a period of 2 hours. The reaction was stirred for a further 1 hour at room temperature and a further aliquot of N-bromosuccinimide (2 g, 11 mmol) was added. The reaction was stirred for a further 1 hour at room temperature. The solvents were evaporated in vacuo and the residue was partitioned between ethyl acetate (200 mL) and water (200 mL). The organic layer was washed with brine (200 mL), dried over anhydrous magnesium sulfate and concentrated in vacuo. The residue was purified by silica gel column chromatography on eluting with 10% EtOAc in heptanes to afford the title compound as a yellow oil (22 g, 57%).

¹H NMR (400 MHz, CDCl₃): δ ppm 5.55 (s, 1H), 7.10 (d, 1H), 7.45 (d, 1H), 7.50-7.60 (m, 2H), 7.60-7.70 (m, 2H), 7.75 (s, 1H).

¹⁹F NMR (400 MHz, CDCl₃): δ ppm −63.0 (s, 3F).

LCMS Rt=2.72 minutes MS m/z 318 [M+H]⁺

### Preparation 40

### 3′-(Trifluoromethyl)biphenyl-4-ol

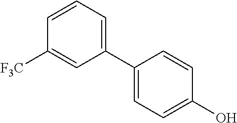 [SMILES: Oc1ccc(-c2cccc(C(F)(F)F)c2)cc1]

4-bromophenol (38.9 g, 173 mmol), (3-(trifluoromethyl)phenyl)boronic acid (25 g, 181 mmol) and sodium carbonate (57.5 g, 543 mmol) were combined and dissolved in a mixture of dioxane/water (4/1, 1100 mL). The reaction was degassed with nitrogen for 20 minutes and tetrakis(triphenylphosphine)palladium(0) (10.0 g, 8.7 mmol) was added in one portion. The reaction was heated at 70° C. for 18 hours, cooled to room temperature and partitioned between ethyl acetate (50 mL) and water (50 mL). The organic layer was washed with brine (50 mL), dried over anhydrous magnesium sulfate and concentrated in vacuo. The residue was purified by silica gel column chromatography eluting with 5-40% EtOAc in heptanes to afford the title compound as a yellow oil (31.0 g, 72%).

¹H NMR (400 MHz, CDCl₃): δ ppm 6.80 (m, 2H), 7.10 (m, 1H), 7.40 (m, 3H), 7.60 (d, 1H), 7.65 (s, 1H).

¹⁹F NMR (400 MHz, CDCl₃): δ ppm −63.0 (s, 3F).

LCMS Rt=2.56 minutes MS m/z 237 [M−H]⁻

### Preparation 41

### tert-Butyl ({4-[4-hydroxy-3′-(trifluoromethyl)biphenyl-3-yl]pyridin-2-yl}methyl)carbamate

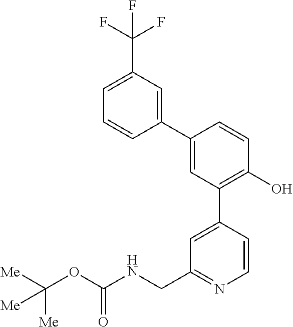 [SMILES: CC(C)(C)OC(=O)NCc1cc(-c2cc(-c3cccc(C(F)(F)F)c3)ccc2O)ccn1]

tert-Butyl ({4-[4-(benzyloxy)-3′-(trifluoromethyl)biphenyl-3-yl]pyridin-2-yl}methyl)carbamate (Preparation 69, 2.66 g, 4.98 mmol) was hydrogenated using palladium hydroxide (266 mg, 10% w/w) in ethanol (30 mL) at 50° C. and 50 psi overnight under hydrogen. The reaction mixture was filtered through a pad of Arbocel and the solvent was evaporated in vacuo. The crude was purified by silica gel column chromatography eluting with 3:2 ethyl acetate:heptane to afford the title compound as a white foam (1.72 g, 78%).

¹H NMR (400 MHz, CDCl₃): δ ppm 1.47 (s, 9H), 4.52 (d, 2H), 5.74 (s, 1H), 7.11 (d, 1H), 7.41-7.43 (dd, 1H), 7.49-7.59 (m, 5H), 7.72 (d, 1H), 7.73 (s, 1H), 8.60 (d, 1H).

¹⁹F NMR (400 MHz, CDCl₃): δ ppm −62.6 (s, 3F).

LCMS Rt=3.43 minutes MS m/z 445 [M+H]⁺

### Preparation 42

### 4-chloro-2-(2-(hydroxymethyl)pyridin-4-yl)phenol

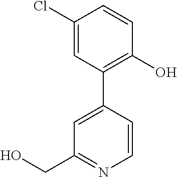 [SMILES: OCc1cc(-c2cc(Cl)ccc2O)ccn1]

To a suspension of 4-(5-chloro-2-hydroxyphenyl)picolinic acid hydrochloride (Preparation 43, 23.1 g, 92.53 mmol) in THF (140 mL) was added boron tribromide dropwise (278 mL, 278 mmol) and the reaction was stirred at room temperature for 18 hours. The reaction was quenched by the addition of 4M HCl (100 mL) and stirred at 70° C. for 18 hours. The reaction was diluted with water (100 mL) and extracted with TBME (2×400 mL). The aqueous layer was collected, basified to pH=7 with 2M NaOH (aq) and the resulting precipitate collected by filtration. The solid was triturated with MeOH to afford the title compound (12.89 g, 59%).

¹H NMR (400 MHz, DMSO-d₆): δ ppm 4.60 (d, 2H), 5.40 (t, 1H), 7.00 (d, 1H), 7.25 (m, 1H), 7.35 (s, 1H), 7.45 (d, 1H), 7.60 (s, 1H), 8.45 (d, 1H), 10.15 (br s, 1H).

MS m/z 236 [M+H]⁺

### Preparation 43

### 4-(5-chloro-2-hydroxyphenyl)picolinic acid hydrochloride salt

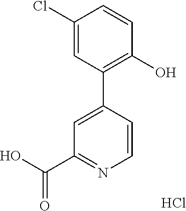 [SMILES: O=C(O)c1cc(-c2cc(Cl)ccc2O)ccn1 ; Cl]

A solution of (5-chloro-2-hydroxyphenyl)boronic acid (16.85 g, 97.7 mmol), 4-bromopicolinic acid (19.74 g, 97.7 mmol) and sodium carbonate (41.43 g, 39.1 mmol) in dioxane (300 mL) and water (120 mL) was degassed with nitrogen before the addition of tetrakistriphenylphosphine palladium (0) (11.3 g, 9.77 mmol) and the reaction was heated to reflux for 18 hours. The reaction was cooled and quenched by the addition of 2M NaOH (aq) until pH>10. The reaction was filtered through Celite and extracted with TBME (2×250 mL). The aqueous layer was acidified to pH=7 using 3M HCl (aq) and the resulting precipitate was collected by filtration. The solid was suspended in water (200 mL), treated with 2M HCl (aq) (250 mL) and stirred for 30 minutes. The precipitate was collected and dried in vacuo azeotroping with MeOH and MeCN to afford the title compound.

¹H NMR (400 MHz, DMSO-d₆): δ ppm 7.00 (d, 1H), 7.30 (m, 1H), 7.45 (m, 1H), 7.80 (m, 1H), 8.25 (m, 1H), 8.70 (d, 1H), 10.40 (br s, 1H).

MS m/z 250 [M+H]⁺

### Preparation 44

### tert-butyl (2,5,8,11,14,17,20,23,26,29,32,35-dodecaoxaheptatriacontan-37-yl)((4-(4-methoxy-3′-(trifluoromethyl)-[1,1′-biphenyl]-3-yl)pyridin-2-yl)methyl)carbamate

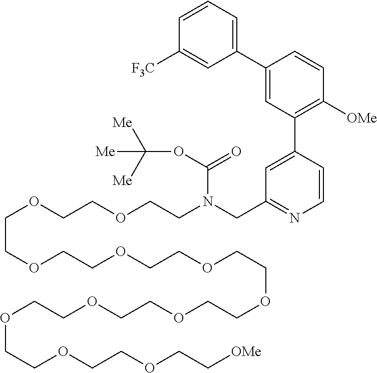 [SMILES: COCCOCCOCCOCCOCCOCCOCCOCCOCCOCCOCCOCCN(Cc1cc(-c2cc(-c3cccc(C(F)(F)F)c3)ccc2OC)ccn1)C(=O)OC(C)(C)C]

To a solution of N-((4-(4-methoxy-3′-(trifluoromethyl)-[1,1′-biphenyl]-3-yl)pyridin-2-yl)methyl)-2,5,8,11,14,17,20,23,26,29,32,35-dodecaoxaheptatriacontan-37-amine (Preparation 46, 170 mg, 0.189 mmol) in DCM (3 mL) was added di-tertbutyldicarbonate (83 mg, 0.378 mmol) and triethylamine (80 uL). The reaction was stirred at room temperature for 18 hours. The reaction was partitioned between water and EtOAc, the organic layer was collected, washed with brine, dried over sodium sulphate and concentrated in vacuo to afford the title compound that was used directly in the next step.

### Preparation 45

### tert-butyl ((4-(4-methoxy-3′-(trifluoromethyl)-[1,1′-biphenyl]-3-yl)pyridin-2-yl)methyl)(2,5,8,11-tetraoxatridecan-13-yl)carbamate

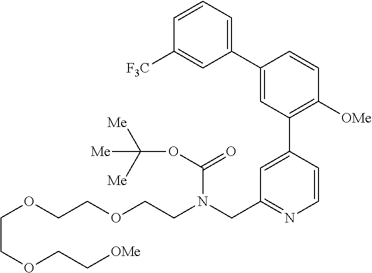 [SMILES: COCCOCCOCCOCCN(Cc1cc(-c2cc(-c3cccc(C(F)(F)F)c3)ccc2OC)ccn1)C(=O)OC(C)(C)C]

To a solution of N-((4-(4-methoxy-3′-(trifluoromethyl)-[1,1′-biphenyl]-3-yl)pyridin-2-yl)methyl)-2,5,8,11-tetraoxatridecan-13-amine (Preparation 47, 65 mg, 0.12 mmol) in DCM (1.5 mL) was added di-tertbutyldicarbonate (52 mg, 0.236 mmol) and triethylamine (49 uL). The reaction was stirred at room temperature for 18 hours. The reaction was partitioned between water and EtOAc, the organic layer was collected, washed with brine, dried over sodium sulphate and concentrated in vacuo to afford the title compound that was used directly in the next step.

### Preparation 46

### N-((4-(4-methoxy-3′-(trifluoromethyl)-[1,1′-biphenyl]-3-yl)pyridin-2-yl)methyl)-2,5,8,11,14,17,20,23,26,29,32,35-dodecaoxaheptatriacontan-37-amine

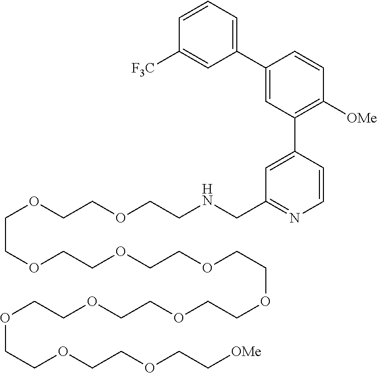 [SMILES: COCCOCCOCCOCCOCCOCCOCCOCCOCCOCCOCCOCCNCc1cc(-c2cc(-c3cccc(C(F)(F)F)c3)ccc2OC)ccn1]

To a solution of 4-(4-methoxy-3′-(trifluoromethyl)-[1,1′-biphenyl]-3-yl)picolinaldehyde (Preparation 51, 150 mg, 0.42 mmol) in DCE (4 mL) was added 2,5,8,11,14,17,20,23,26,29,32,35-dodecaoxaheptatriacontan-37-amine (m-dPEG12-NH₂, 235 mg, 0.42 mmol) and acetic acid (72 uL). The reaction was stirred at room temperature for 1.5 hours. Sodium triacetoxyborohydride (187 mg, 0.84 mmol) was added and the reaction stirred at room temperature for 18 hours. The reaction was quenched by the addition of MeOH (0.5 mL) followed by saturated aqueous NaHCO₃ solution and extracted into DCM twice. The organic layers were collected, dried over sodium sulphate and concentrated in vacuo. The residue was purified using silica gel column chromatography eluting with 0-10% MeOH in DCM to afford the title compound (173 mg, 46%).

¹H NMR (400 MHz, CDCl₃): δ ppm 3.37 (s, 3H), 3.54-3.72 (m, 46H), 3.88 (s, 3H), 4.05 (s, 2H), 7.10 (d, 1H), 7.37-7.44 (m, 1H), 7.51-7.59 (m, 4H), 7.59-7.64 (m, 1H), 7.71-7.79 (m, 1H), 7.78-7.83 (m, 1H), 8.56-8.62 (m, 1H).

### Preparation 47

### N-((4-(4-methoxy-3′-(trifluoromethyl)-[1,1′-biphenyl]-3-yl)pyridin-2-yl)methyl)-2,5,8,11-tetraoxatridecan-13-amine

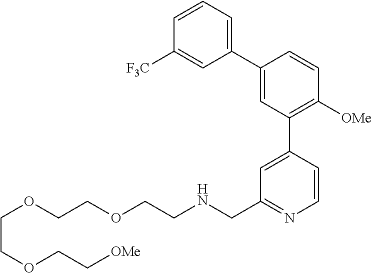 [SMILES: COCCOCCOCCOCCNCc1cc(-c2cc(-c3cccc(C(F)(F)F)c3)ccc2OC)ccn1]

To a solution of 4-(4-methoxy-3′-(trifluoromethyl)-[1,1′-biphenyl]-3-yl)picolinaldehyde (Preparation 51, 83 mg, 0.23 mmol) in DCE (2.5 mL) was added 2,5,8,11-tetraoxatridecan-13-amine (m-dPEG4-NH₂, 49 mg, 0.23 mmol) and acetic acid (40 uL). The reaction was stirred at room temperature for 1.5 hours. Sodium triacetoxyborohydride (104 mg, 0.464 mmol) was added and the reaction stirred at room temperature for 18 hours. The reaction was quenched by the addition of MeOH (0.5 mL) followed by saturated aqueous NaHCO₃ solution and extracted into DCM twice. The organic layers were collected, dried over sodium sulphate and concentrated in vacuo. The residue was purified using silica gel column chromatography eluting with 0-10% MeOH in DCM to afford the title compound (67 mg, 53%).

¹H NMR (400 MHz, CDCl₃): δ ppm 3.40 (s, 3H), 3.55 (m, 2H), 3.60-3.70 (m, 14H), 3.90 (s, 3H), 4.00 (s, 2H), 7.10 (m, 1H), 7.20 (m, 1H), 7.55-7.65 (m, 5H), 7.75 (m, 1H), 7.80 (m, 1H), 8.60 (m, 1H).

LCMS Rt=1.48 minutes MS m/z 549 [M+H]⁺

### Preparation 48

### 1-((4-(4-methoxy-3′-(trifluoromethyl)-[1,1′-biphenyl]-3-yl)pyridin-2-yl)methyl)-3-(25,8,11-tetraoxatridecan-13-yl)urea

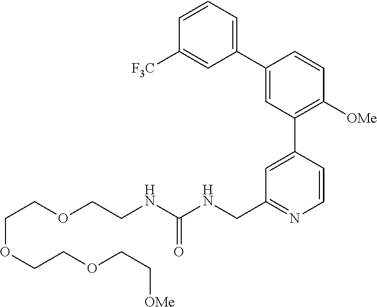 [SMILES: COCCOCCOCCOCCNC(=O)NCc1cc(-c2cc(-c3cccc(C(F)(F)F)c3)ccc2OC)ccn1]

To a solution of (4-(4-methoxy-3′-(trifluoromethyl)-[1,1′-biphenyl]-3-yl)pyridin-2-yl)methanamine (Preparation 49, 60 mg, 0.17 mmol) in DCM (0.5 mL) was added 4-nitrophenyl (2,5,8,11-tetraoxatridecan-13-yl)carbamate (Preparation 67, 60 mg, 0.16 mmol) and the reaction was stirred at room temperature for 2.5 hours. The reaction was quenched by the addition of water and extracted into EtOAc. The organic layer was collected, dried over sodium sulfate and concentrated in vacuo. The residue was purified using silica gel column chromatography eluting with 0-10% MeOH in DCM to afford the title compound (41 mg, 43%).

¹H NMR (400 MHz, CDCl₃): δ ppm 3.35 (s, 3H), 3.40 (m, 2H), 3.50-3.70 (m, 16H), 3.90 (s, 3H), 4.60 (s, 2H), 5.55 (br s, 1H), 5.90 (br s, 1H), 7.10 (m, 1H), 7.40 (m, 1H), 7.50-7.60 (m, 6H), 7.75 (m, 1H), 7.80 (m, 1H), 8.60 (m, 1H).

### Preparation 49

### (4-(4-methoxy-3′-(trifluoromethyl)-[1,1′-biphenyl]-3-yl)pyridin-2-yl)methanamine

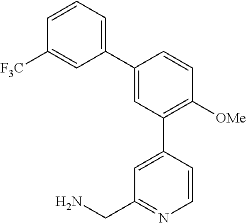 [SMILES: COc1ccc(-c2cccc(C(F)(F)F)c2)cc1-c1ccnc(CN)c1]

To a solution of 2-(chloromethyl)-4-(4-methoxy-3′-(trifluoromethyl)-[1,1′-biphenyl]-3-yl)pyridine hydrochloride (Preparation 50, 45 mg, 0.108 mol) in DMF (200 mL) was added potassium carbonate (45 g, 0.326 mol) followed by phthalimide (32 g, 0.218 mol). The reaction was heated at 40° C. for 18 hours. Further potassium carbonate (45 g, 0.326 mol) and phthalimide (15 g, 0.102 mol) were added and the reaction heated at 50° C. for 5 hours. The reaction was partitioned between TMBE (800 mL) and water (800 mL). The organic layer was collected, washed with water (250 mL), dried over sodium sulphate and concentrated in vacuo. The residue was recrystallised from MeOH and added portionwise to a solution of methylamine in water (40% w/v, 500 mL) and stirred at room temperature for 4 hours. Excess methylamine was removed in vacuo and the residue treated with water (150 mL) and 2M NaOH (aq) (50 mL), and extracted into TBME (300 mL). The organic layer was collected, dried over sodium sulphate and concentrated in vacuo, azeotroping with toluene. The residue was triturated with heptanes (100 mL) and TBME (50 mL) to afford the title compound (16.8 g, 68%).

¹H NMR (400 MHz, MeOH-d₄): δ ppm 3.89 (s, 3H), 3.96 (s, 2H), 7.25 (d, 1H), 7.54 (d, 1H), 7.61 (m, 2H), 7.67 (m, 2H), 7.72 (m, 1H), 7.89 (m, 2H), 8.49 (d, 1H).

¹⁹F NMR (376 MHz, MeOH-d₄): δ ppm −64.0 (s, 3F).

### Preparation 50

### 2-(chloromethyl)-4-(4-methoxy-3′-(trifluoromethyl)-[1,1′-biphenyl]-3-yl)pyridine hydrochloride salt

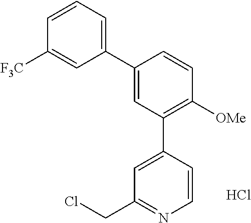 [SMILES: COc1ccc(-c2cccc(C(F)(F)F)c2)cc1-c1ccnc(CCl)c1 ; Cl]

To a solution of (4-(4-methoxy-3′-(trifluoromethyl)-[1,1′-biphenyl]-3-yl)pyridin-2-yl)methanol (Preparation 57, 38.8 g, 0.108 mol) in DCM at 0° C. was added thionyl chloride (12 mL, 0.164 mol) dropwise over 30 minutes. The reaction was warmed to room temperature over 2.5 hours. Toluene (50 mL) was added and the solution concentrated in vacuo, azeotroping with further toluene to afford the title compound (44.7 g, 100%).

¹H NMR (400 MHz, DMSO-d₆): δ ppm 3.87 (s, 3H), 4.92 (s, 2H), 7.32 (d, 1H), 7.67 (m, 2H), 7.80 (m, 1H), 7.85-7.87 (m, 2H), 8.02 (m, 3H), 8.71 (d, 1H).

### Preparation 51

### 4-(4-methoxy-3′-(trifluoromethyl)-[1,1′-biphenyl]-3-yl)picolinaldehyde

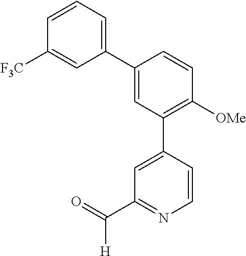 [SMILES: COc1ccc(-c2cccc(C(F)(F)F)c2)cc1-c1ccnc(C=O)c1]

To a solution of (4-(4-methoxy-3′-(trifluoromethyl)-[1,1′-biphenyl]-3-yl)pyridin-2-yl)methanol (Preparation 57, 140 mg, 0.39 mmol) in DCM (4 mL) was added Dess Martin reagent (192 mg, 0.429 mmol) and the reaction was stirred at room temperature for 2 hours. Saturated aqueous NaHCO₃ solution and saturated aqueous sodium thiosulfate solution were added with stirring for 30 minutes. The organic layer was separated, the aqueous was washed with further DCM, the organic layers were combined, dried over sodium sulphate and concentrated in vacuo to afford the title compound (150 mg, quant).

¹H NMR (400 MHz, CDCl₃): δ ppm 3.92 (s, 3H), 7.14 (d, 1H), 7.53-7.63 (m, 3H), 7.67 (m, 1H), 7.75-7.80 (m, 2H), 7.82 (s, 1H), 8.21 (m, 1H), 8.84 (m, 1H), 10.17 (s, 1H).

### Preparation 52

### 4-(4-methoxy-3′-(trifluoromethyl)-[1,1′-biphenyl]-3-yl)-2-(2,5,8,11,14-pentaoxapentadecyl)pyridine

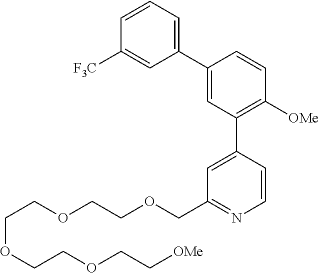 [SMILES: COCCOCCOCCOCCOCc1cc(-c2cc(-c3cccc(C(F)(F)F)c3)ccc2OC)ccn1]

To a solution of (4-(4-methoxy-3′-(trifluoromethyl)-[1,1′-biphenyl]-3-yl)pyridin-2-yl)methanol (Preparation 57, 75 mg, 0.209 mmol) and 2,5,8,11-tetraoxatridecan-13-yl 4-methylbenzenesulfonate (m-dPEG4-OTf, 76 mg, 0.209 mmol) in DMF (2.3 mL) was added sodium hydride (25 mg, 0.627 mmol) followed by sodium iodide (2.9 mg, 0.021 mmol) and the reaction was stirred at room temperature for 1 hour. The reaction was diluted with DCM and water, the organic layer was collected and the aqueous layer washed with further DCM. The organic layers were collected and combined, dried over sodium sulphate and concentrated in vacuo. The residue was purified using silica gel column chromatography eluting with 0-10% MeOH in DCM to afford the title compound (67 mg, 58%).

¹H NMR (400 MHz, CDCl₃): δ ppm 3.40 (s, 3H), 3.55-3.80 (m, 16H), 3.90 (s, 3H), 4.80 (s, 2H), 7.10 (d, 1H), 7.40 (m, 1H), 7.60-7.70 (m, 5H), 7.75 (m, 1H), 7.80 (m, 1H), 8.60 (m, 1H).

MS m/z 550 [M+H]⁺

### Preparation 53

### N-(2-((4-(4-methoxy-3′-(trifluoromethyl)-[1,1′-biphenyl]-3-yl)pyridin-2-yl) methoxy)ethyl)-2,5,8,11-tetraoxatridecan-13-amine

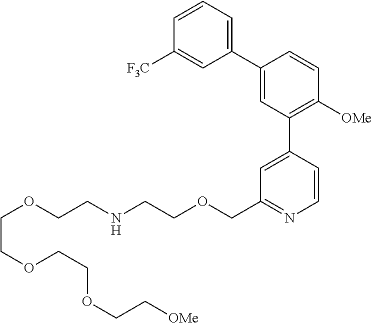 [SMILES: COCCOCCOCCOCCNCCOCc1cc(-c2cc(-c3cccc(C(F)(F)F)c3)ccc2OC)ccn1]

To a solution of 2-((4-(4-methoxy-3′-(trifluoromethyl)-[1,1′-biphenyl]-3-yl)pyridin-2-yl)methoxy)-N-(2,5,8,11-tetraoxatridecan-13-yl)acetamide (Preparation 54, 20 mg, 0.033 mmol) in THF (0.2 mL) was added borane-dimethylsulfide (0.02 mL, 0.099 mmol) dropwise and the reaction was stirred at room temperature for 10 minutes followed by heating to 65° C. under microwave irradiation for 15 minutes. The reaction was partitioned between EtOAc and water, the organic layer was collected, washed with brine, dried over sodium sulphate and concentrated in vacuo. The residue was purified using silica gel column chromatography eluting with 0-10% MeOH in DCM with 3% triethylamine to afford the title compound (10 mg, 51%) and taken directly on to the next step.

### Preparation 54

### 2-((4-(4-methoxy-3′-(trifluoromethyl)-[1,1′-biphenyl]-3-yl)pyridin-2-yl) methoxy)-N-(2,5,8,11-tetraoxatridecan-13-yl)acetamide

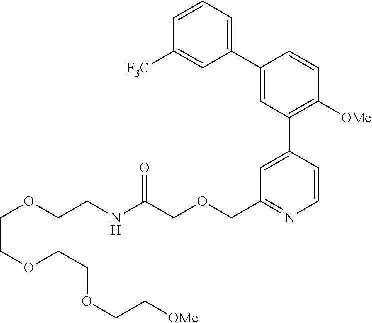 [SMILES: COCCOCCOCCOCCNC(=O)COCc1cc(-c2cc(-c3cccc(C(F)(F)F)c3)ccc2OC)ccn1]

To a solution of 2-((4-(4-methoxy-3′-(trifluoromethyl)-[1,1′-biphenyl]-3-yl)pyridin-2-yl)methoxy)acetic acid (Preparation 55, 90 mg, 0.22 mmol) in DMF (2 mL) was added 2,5,8,11-tetraoxatridecan-13-amine (m-dPEG4-NH₂, 45 mg, 0.126 mmol) was added DIPEA (0.157 mL, 0.864 mmol) followed by COMU® (111 mg, 0.259 mmol) and the reaction was stirred at room temperature for 18 hours. The reaction was diluted with water and extracted into EtOAc twice. The combined organic layers were washed with brine, dried over sodium sulphate and concentrated in vacuo. The residue was purified using silica gel column chromatography eluting with 0-10% MeOH in DCM to afford the title compound (83 mg, 63%).

MS m/z 607 [M+H]⁺

### Preparation 55

### 2-((4-(4-methoxy-3′-(trifluoromethyl)-[1,1′-biphenyl]-3-yl)pyridin-2-yl)methoxy)acetic acid

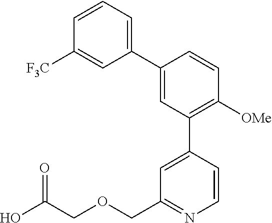 [SMILES: COc1ccc(-c2cccc(C(F)(F)F)c2)cc1-c1ccnc(COCC(=O)O)c1]

To a solution of tert-butyl 2-((4-(4-methoxy-3′-(trifluoromethyl)-[1,1′-biphenyl]-3-yl)pyridin-2-yl)methoxy)acetate (Preparation 56, 111 mg, 0.234 mmol) in DCM (2 mL) was added TFA (0.361 mL, 4.68 mmol) and the reaction was stirred at room temperature for 1 hour. The reaction was concentrated in vacuo azeotroping with EtOAc, heptanes and DCM to afford the title compound that was used directly in the next step.

MS m/z 418 [M+H]⁺

### Preparation 56

### tert-butyl 2-((4-(4-methoxy-3′-(trifluoromethyl)-[1,1′-biphenyl]-3-yl)pyridin-2-yl) methoxy)acetate

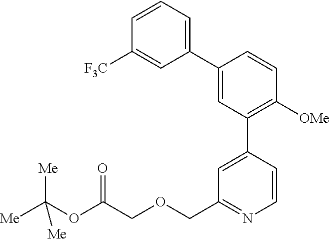 [SMILES: COc1ccc(-c2cccc(C(F)(F)F)c2)cc1-c1ccnc(COCC(=O)OC(C)(C)C)c1]

To a 0° C. solution of (4-(4-methoxy-3′-(trifluoromethyl)-[1,1′-biphenyl]-3-yl)pyridin-2-yl)methanol (Preparation 57, 140 mg, 0.39 mmol) in DMF (4 mL) was added sodium hydride (47 mg, 1.17 mmol) and the reaction was stirred at this temperature for 40 minutes. Tert-butylbromoacetate (63 uL, 0.429 mmol) was then added and the reaction stirred at room temperature for 18 hours. The reaction was quenched with water (20 mL) and extracted into EtOAc. The organic layer was collected, washed with brine, dried over sodium sulphate and concentrated in vacuo. The residue was purified using silica gel column chromatography eluting with 0-50% EtOAc in heptanes to afford the title compound (110 mg, 60%) that was taken directly on to the next step.

### Preparation 57

### (4-(4-methoxy-3′-(trifluoromethyl)-[1,1′-biphenyl]-3-yl)pyridin-2-yl)methanol

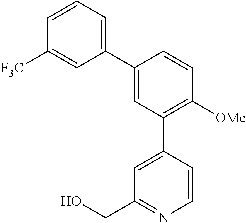 [SMILES: COc1ccc(-c2cccc(C(F)(F)F)c2)cc1-c1ccnc(CO)c1]

To a solution of 4-(4-methoxy-3′-(trifluoromethyl)-[1,1′-biphenyl]-3-yl)-2-methylpyridine (Preparation 58, 74.2 g, 216 mol) in DCM (740 mL) at 5° C. was added mCPBA (70% wt active, 93.2 g, 378 mol) portionwise and the reaction was stirred at room temperature for 18 hours. Further mCPBA (10 g, 82 mmol) was added, with further stirring for 2 hours before quenching with saturated aqueous NaHCO₃ solution (300 mL). The organic layer was collected, the aqueous layer backwashed with DCM (200 mL), the organic layers were combined, washed with saturated aqueous NaHCO₃ solution (3×900 mL), filtered through a phase separation cartridge and concentrated in vacuo. The residue was dissolved in DCM (340 mL), cooled to 5° c. and treated with TFAA (340 mL, 2.41 mol). The reaction was heated to reflux for 45 hours before cooling and concentrating in vacuo. The residue was dissolved in DCM (700 mL), cooled to 5° C. and treated with 2M NaOH (aq) (350 mL) with stirring for 18 hours. DCM (200 mL) followed by 1M NaOH (aq) (350 mL) was added and the organic layer collected. The aqueous layer was extracted with DCM (2×250 mL) and the organic layers combined, dried over magnesium sulphate and concentrated in vacuo. The residue was purified using silica gel column chromatography eluting with EtOAc to afford the title compound (46.2 g, 66%).

¹H NMR (400 MHz, CDCl₃): δ ppm 3.90 (s, 3H), 4.84 (s, 2H), 7.12 (d, 1H), 7.45 (dd, 1H), 7.47 (s, 1H), 7-54-7.62 (m, 3H), 7.64 (dd, 1H), 7.76 (d, 1H), 7.82 (s, 1H), 8.62 (d, 1H).

MS m/z 360 [M+H]⁺

### Preparation 58

### 4-(4-methoxy-3′-(trifluoromethyl)-[1,1′-biphenyl]-3-yl)-2-methylpyridine

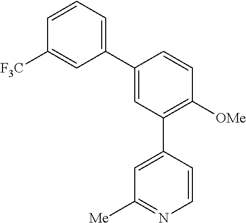 [SMILES: COc1ccc(-c2cccc(C(F)(F)F)c2)cc1-c1ccnc(C)c1]

To a mixture of 4-(5-chloro-2-methoxyphenyl)-2-methylpyridine (Preparation 59, 55 g, 0.24 mol), 4,4,5,5-tetramethyl-2-(3-(trifluoromethyl)phenyl)-1,3,2-dioxaborolane (104.8 g, 0.35 mol) and potassium carbonate (97.4 g, 0.71 mmol) was added 2-methyl-2-butanol (660 mL) and water (385 mL). The reaction mixture was sparged with nitrogen and degassed in vacuo before the addition of palladium acetate (1.06 g, 470 mmol) and XPhos (4.48 g, 9.40 mmol). The reaction was heated to 100° C. for 18 hours, then cooled, diluted with EtOAc (200 mL) and filtered through Celite. The filtrate was separated, the organic layer collected and concentrated in vacuo. The residue was purified using silica gel column chromatography eluting with 5-100% EtOAc in heptanes. The residue was dissolved in EtOAc (250 mL) and extracted into 1M HCl (aq) (2×500 mL). The aqueous layers were combined and basified by the addition of concentrated aqueous NaOH solution. The product was extracted into EtOAc (2×200 mL), the organic layers combined, dried over magnesium sulphate and concentrated in vacuo to afford the title compound (74.2 g, 81%).

¹H NMR (400 MHz, CDCl₃): δ ppm 2.64 (s, 3H), 3.90 (s, 3H), 7.11 (d, 1H), 7.34 (dd, 1H), 7.37 (s, 1H), 7.55 (d, 1H), 7.61-7.55 (m, 2H), 7.62 (dd, 1H), 7.76 (d, 1H), 7.82 (s, 1H), 8.59 (d, 1H).

### Preparation 59

### 4-(5-chloro-2-methoxyphenyl)-2-methylpyridine

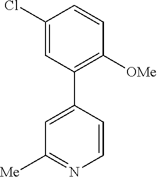 [SMILES: COc1ccc(Cl)cc1-c1ccnc(C)c1]

To a mixture of (5-chloro-2-methoxyphenyl)boronic acid (56.89 g, 0.31 mol), 4-bromo-2-methylpyridine (50 g, 0.29 mol) and sodium carbonate (98.57 g, 0.91 mol) was added 1,4, dioxane (0.9 L) and water (0.18 L). The reaction was sparged with nitrogen and degassed in vacuo. Pd(dppf)C₂ (12.66 g, 16 mmol) was added and the reaction heated to 100° C. for 18 hours. The reaction was cooled to room temperature, filtered through Celite and the filtrate was concentrated in vacuo. The residue was purified using silica gel column chromatography eluting with EtOAc. The residue was dissolved in EtOAc and extracted into 1M HCl (2×2 L). The aqueous layers were combined, basified with concentrated aqueous NaOH solution and extracted into EtOAc (2×750 mL). The organic layers were combined, washed with brine (300 mL), dried over magnesium sulphate and concentrated in vacuo to afford the title compound (64.5 g, 95%).

¹H NMR (400 MHz, CDCl₃): δ ppm 2.60 (s, 3H), 3.81 (s, 3H), 6.91 (d, 1H), 7.28 (m, 4H), 8.51 (d, 1H).

### Preparation 60

### tert-Butyl 4-(2-{[(4-bromopyridin-2-yl)methyl][(2,2,2-trichloroethoxy)carbonyl]amino}ethyl)piperidine-1-carboxylate

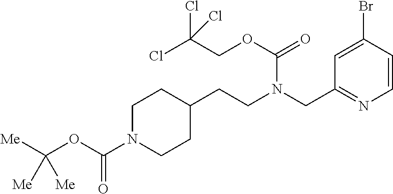 [SMILES: CC(C)(C)OC(=O)N1CCC(CCN(Cc2cc(Br)ccn2)C(=O)OCC(Cl)(Cl)Cl)CC1]

To a solution of tert-butyl 4-(2-{[(4-bromopyridin-2-yl)methyl]amino}ethyl)piperidine-1-carboxylate (Preparation 61, 5.76 g, 14.6 mmol) in dichloromethane (75 mL) was added triethylamine (3.06 mL, 21.8 mmol) followed by 2,2,2-trichloroethylchloroformate (2.21 mL, 16.0 mmol) at room temperature. The reaction was stirred at room temperature for 1 hour. An aqueous solution of ammonium chloride (100 mL) was added followed by water (100 mL) and the aqueous phase was extracted with dichloromethane (3×100 mL). The combined organic layers were washed with brine (50 mL), dried over anhydrous magnesium sulfate and concentrated in vacuo to afford the title compound as a yellow oil (9.51 g, >100%). No further purification undertaken.

¹H NMR (400 MHz, CDCl₃): δ ppm 1.05 (m, 2H), 1.40 (s, 9H), 1.50-1.70 (m, 5H), 2.60 (m, 2H), 3.40 (m, 2H), 4.00 (m, 2H), 4.60 (s, 2H), 4.75 (s, 2H), 7.35 (m, 1H), 7.50 (m, 1H), 8.35 (d, 1H).

LCMS Rt=3.06 minutes MS m/z 573 [M+H]⁺

### Preparation 61

### tert-Butyl 4-(2-{[(4-bromopyridin-2-yl)methyl]amino}ethyl)piperidine-1-carboxylate

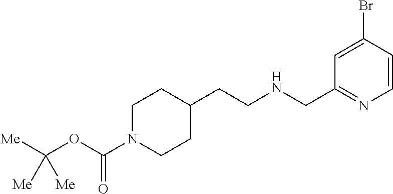 [SMILES: CC(C)(C)OC(=O)N1CCC(CCNCc2cc(Br)ccn2)CC1]

To a solution of (4-bromopyridin-2-yl)methanamine (2.86 g, 15.1 mmol) in methanol (75 mL) was added tert-butyl 4-(2-oxoethyl)piperidine-1-carboxylate (3.44 g, 15.1 mmol). The reaction was stirred at room temperature for 18 hours and cooled to 0° C. in an ice bath. Sodium borohydride (173 g, 45.4 mmol) was added portionwise and after 30 minutes at room temperature, the mixture was quenched by addition of water (50 mL). The aqueous phase was extracted with ethyl acetate (3×100 mL) and the combined organic layers were washed with brine (100 mL), dried over anhydrous magnesium sulfate, filtered and concentrated in vacuo to afford the title compound as a light yellow oil (5.76 g, 95%).

¹H NMR (400 MHz, CDCl₃): δ ppm 1.05 (m, 2H), 1.40 (s, 9H), 1.50 (m, 2H), 1.60 (m, 2H), 1.70 (m, 1H), 2.60 (m, 4H), 3.90 (s, 2H), 4.10 (br s, 2H), 7.30 (dd, 1H), 7.50 (s, 1H), 8.30 (d, 1H).

LCMS Rt=1.91 minutes MS m/z 399 [M+H]⁺

### Preparation 62

### tert-Butyl 4-(2-{[(benzyloxy)carbonyl][(4-bromopyridin-2-yl)methyl]amino}ethyl)piperidine-1-carboxylate

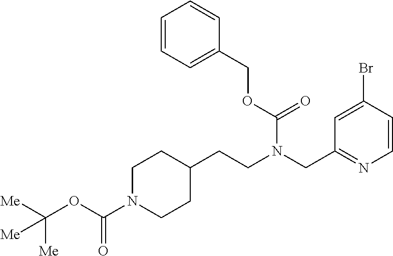 [SMILES: CC(C)(C)OC(=O)N1CCC(CCN(Cc2cc(Br)ccn2)C(=O)OCc2ccccc2)CC1]

Triethylamine (1.47 mL, 10.6 mmol) and benzyl chloroformate (0.9 mL, 6.33 mmol) were added to a solution of tert-butyl 4-(2-{[(4-bromopyridin-2-yl)methyl]amino}ethyl)piperidine-1-carboxylate (Preparation 61, 2.10 g, 5.28 mmol) in dichloromethane (25 mL) at 0° C. The reaction mixture was stirred for 1 hour at room temperature, washed with water (30 mL), and the aqueous layer was extracted with dichloromethane (50 mL). The combined organic layers were dried over magnesium sulfate. The filtrate was concentrated in vacuo and the residue was purified by silica gel column chromatography eluting with 2:3 ethyl acetate:cyclohexane to afford the title compound as an oil (2.54 g, 90%).

¹H NMR (400 MHz, CDCl₃): δ ppm 0.96-1.13 (m, 2H), 1.31-1.66 (m, 14H), 2.48-2.66 (m, 2H), 3.33-3.43 (m, 2H), 4.00 (br s, 2H), 4.57 (d, 2H), 5.19 (d, 2H), 7.24-7.46 (m, 7H), 8.33 (d, 1H).

LCMS Rt=2.96 minutes MS m/z 533 [M+H]⁺

### Preparation 63

### tert-butyl 3-((2,5,8,11-tetraoxatridecan-13-yl)oxy)azetidine-1-carboxylate

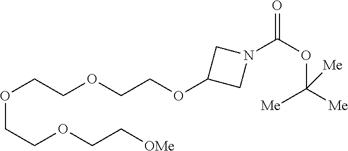 [SMILES: COCCOCCOCCOCCOC1CN(C(=O)OC(C)(C)C)C1]

To a solution of tert-butyl 3-hydroxyazetidine-1-carboxylate (173 mg, 0.99 mmol) in THF (2 mL) was added sodium hydride (52 mg, 1.30 mmol) and the reaction was stirred at room temperature for 20 minutes. 1-bromo-2,5,8,11-tetraoxadodecane (m-dPEG4-Br, 271 mg, 0.99 mmol) was added and the reaction stirred at room temperature for 18 hours. The reaction was quenched by the addition of water and extracted into EtOAc. The organic layer was collected, dried over sodium sulfate and concentrated in vacuo. The residue was purified using silica gel column chromatography eluting with 0-100% EtOAc in heptanes to afford the title compound.

¹H NMR (400 MHz, DMSO-d₆): δ ppm 1.37 (s, 9H), 3.25 (s, 3H), 3.41-3.51 (m, 16H), 3.61-3.65 (m, 2H), 3.96-4.01 (m, 2H), 4.22-4.26 (m, 1H).

MS m/z 364 [M+H]⁺

### Preparation 64

### (R)-3-((2,5,8,11-tetraoxatridecan-13-yl)oxy)pyrrolidine trifluoroacetate salt

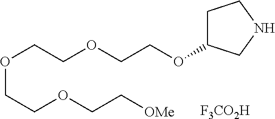

The Boc-protected title compound was prepared according to the method described for Preparation 63 using tert-butyl (R)-3-hydroxypyrrolidine-1-carboxylate and 1-bromo-2,5,8,11-tetraoxadodecane (m-dPEG4-Br). The Boc intermediate was dissolved in TFA (0.5 mL) and heated to 50° C. for 2 hours. The reaction was concentrated in vacuo and purified using silica gel column chromatography eluting with 0-10% MeOH in DCM to afford the title compound.

¹H NMR (400 MHz, DMSO-d₆): δ ppm 1.93 (m, 2H), 3.09 (m, 4H), 3.32 (s, 3H), 3.44 (m, 3H), 3.49-3.51 (m, 13H), 4.16 (m, 1H), 8.03 (br s, 1H).

MS m/z 278 [M+H]⁺

### Preparation 65

### (S)-3-((2,5,8,11-tetraoxatridecan-13-yl)oxy)pyrrolidine trifluoroacetate salt

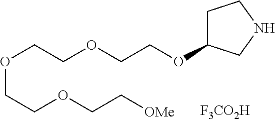

The Boc-protected title compound was prepared according to the method described for Preparation 63 using tert-butyl (S)-3-hydroxypyrrolidine-1-carboxylate and 1-bromo-2,5,8,11-tetraoxadodecane (m-dPEG4-Br). The Boc intermediate was dissolved in TFA (0.5 mL) and heated to 50° C. for 2 hours. The reaction was concentrated in vacuo and purified using silica gel column chromatography eluting with 0-10% MeOH in DCM to afford the title compound.

¹H NMR (400 MHz, DMSO-d₆): δ ppm 1.99 (br m, 2H), 3.18-3.32 (m, 7H), 3.44-3.53 (m, 16H), 4.21 (m, 1H), 8.76 (br s, 1H).

MS m/z 278 [M+H]⁺

### Preparation 66

### 4-((2,5,8,11-tetraoxatridecan-13-yl)oxy)piperidine hydrochloride salt

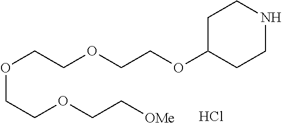 [SMILES: COCCOCCOCCOCCOC1CCNCC1 ; Cl]

The Boc-protected title compound was prepared according to the method described for Preparation 63 using tert-butyl-4-hydroxypiperidine-1-carboxylate and triethyleneglycol-2-bromoethylmethylether. The Boc intermediate was dissolved in dioxane and treated with 4M HCl in dioxane and stirred at room temperature for 2 hours. Diethyl ether was added to the reaction and the resulting precipitate was collected and triturated with diethyl ether.

¹H NMR (400 MHz, DMSO-d₆): δ ppm 1.60-1.76 (m, 2H), 1.90-2.10 (m, 2H), 2.88-3.10 (m, 2H), 3.08-3.20 (m, 2H), 3.26 (s, 3H), 3.42-3.62 (m, 17H).

MS m/z 292 [M+H]⁺

### Preparation 67

### 4-nitrophenyl (2,5,8,11-tetraoxatridecan-13-yl)carbamate

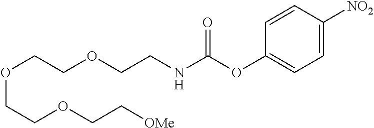 [SMILES: COCCOCCOCCOCCNC(=O)Oc1ccc([N+](=O)[O-])cc1]

To a solution of bis(4-nitrophenyl) carbonate (56 mg, 0.182 mmol) in DCM (1.7 mL) was added DIPEA (51 uL, 0.280 mmol) followed by a solution of 2,5,8,11-tetraoxatridecan-13-amine (m-dPEG4-NH₂, 29 mg, 0.14 mmol) in DCM (0.5 mL) and the reaction was stirred at room temperature for 1 hour. The reaction was partitioned between water and EtOAc, the organic layer was collected, dried over sodium sulphate, concentrated in vacuo and used directly in the next reaction.

### Preparation 68

### 3-(4,4,5,5-Tetramethyl-1,3,2-dioxaborolan-2-yl)-3′-(trifluoromethyl) biphenyl-4-ol

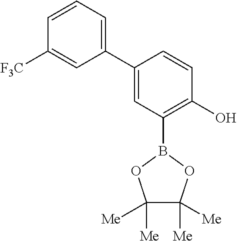 [SMILES: CC1(C)OB(c2cc(-c3cccc(C(F)(F)F)c3)ccc2O)OC1(C)C]

To a solution of 2-[4-(Benzyloxy)-3′-(trifluoromethyl)biphenyl-3-yl]-4,4,5,5-tetramethyl-1,3,2-dioxaborolane (Preparation 70, 16.3 g, 35.9 mmol) in methanol (250 mL) was added 10% palladium on carbon (1.63 g, 10% w/w) and ammonium formate (9.06 g, 143.6 mmol). The reaction mixture was heated at 60° C. for 1 hour. The reaction mixture was cooled and the solvent was evaporated in vacuo. The residue was diluted with dichloromethane (200 mL) and was washed with water (100 mL). The organic layer was dried over magnesium sulfate and concentrated in vacuo to afford the title compound as a solid (11.6 g, 89%).

¹H NMR (400 MHz, CDCl₃): δ ppm 1.40 (s, 12H), 6.98 (d, 1H), 7.53 (d, 1H), 7.62 (dd, 1H), 7.74 (d, 1H), 7.80 (s, 1H), 7.84 (d, 1H), 7.94 (d, 1H).

¹⁹F NMR (400 MHz, CDCl₃): δ ppm −62.5 (s, 3F).

MS m/z No mass ion observed

### Preparation 69

### tert-Butyl ({4-[4-(benzyloxy)-3′-(trifluoromethyl)biphenyl-3-yl]pyridin-2-yl}methyl)carbamate

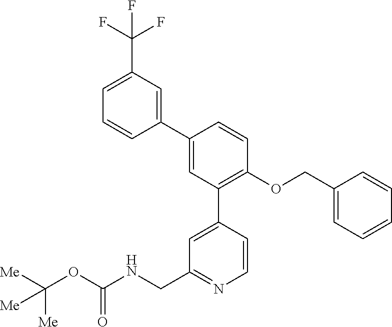 [SMILES: CC(C)(C)OC(=O)NCc1cc(-c2cc(-c3cccc(C(F)(F)F)c3)ccc2OCc2ccccc2)ccn1]

2-[4-(Benzyloxy)-3′-(trifluoromethyl) biphenyl-3-yl]-4,4,5,5-tetramethyl-1,3,2-dioxaborolane (Preparation 70, 3.09 g, 6.69 mmol), tert-butyl [(4-bromopyridin-2-yl)methyl]carbamate (Preparation 72, 1.95 g, 6.97 mmol) and sodium carbonate (2.85 g, 26.87 mmol) in a mixture of dioxane (30 mL) and water (6.0 mL) were degassed. Tetrakis(triphenylphosphine)palladium(0) (0.39 g, 0.34 mmol) was added and the reaction mixture was further degassed and heated at 80° C. for 7 hours. The reaction mixture was cooled to room temperature, diluted with ethyl acetate (50 mL), washed with water (50 mL) and the organic layer was dried over magnesium sulfate and solvent was evaporated in vacuo. The residue was purified by silica gel column chromatography eluting with 1:1 ethyl acetate:heptane to afford the title compound as a glassy solid (2.66 g, 73%).

¹H NMR (400 MHz, CDCl₃): δ ppm 1.46 (s, 9H), 4.50 (d, 2H), 5.16 (s, 2H), 5.55 (br s, 1H), 7.16 (d, 1H), 7.31-7.39 (m, 5H), 7.44 (dd, 1H), 7.51-7.60 (m, 5H), 7.74 (d, 1H), 7.80 (s, 1H), 8.58 (d, 1H).

¹⁹F NMR (400 MHz, CDCl₃): δ −ppm 62.6 (s, 3F).

LCMS Rt=3.98 minutes MS m/z 535 [M+H]⁺

### Preparation 70

### 2-[4-(Benzyloxy)-3′-(trifluoromethyl)biphenyl-3-yl]-4,4,5,5-tetramethyl-1,3,2-dioxaborolane

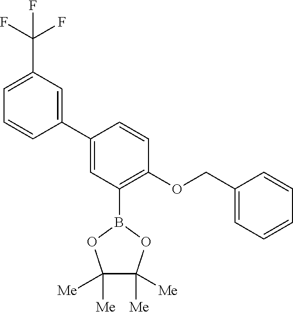 [SMILES: CC1(C)OB(c2cc(-c3cccc(C(F)(F)F)c3)ccc2OCc2ccccc2)OC1(C)C]

4-(Benzyloxy)-3-bromo-3′-(trifluoromethyl)biphenyl (Preparation 71, 25.2 g, 61.9 mmol), Bis(pinacolato)diboron (18.4 g, 74.2 mmol) and potassium acetate (18.2 g, 186 mmol) were combined and dissolved in dimethoxyethane (400 mL). The reaction was degassed with nitrogen for 20 minutes and [1,1′-bis(diphenylphosphino)ferrocene]dichloropalladium(II), complex with dichloromethane (2.49 g, 3.1 mmol) was added in one portion. The reaction was heated at 70° C. for 18 hours, cooled to room temperature, filtered through a pad of celite (eluting with ethyl acetate (200 mL)) and the filtrate partitioned between ethyl acetate and water. The organic layer was washed with brine (50 mL), dried over anhydrous magnesium sulfate and concentrated in vacuo. The crude compound was purified by silica gel column chromatography eluting with 20-80% EtOAc in heptanes to afford the title compound as a light yellow solid (17.8 g, 64%).

¹H NMR (400 MHz, CDCl₃): δ ppm 1.30 (s, 12H), 5.15 (d, 2H), 7.00 (d, 1H), 7.15 (s, 1H), 7.20 (s, 1H), 7.30 (d, 1H), 7.40 (m, 2H), 7.50 (m, 1H), 7.60 (m, 2H), 7.75 (d, 1H), 7.85 (s, 1H), 7.90 (s, 1H).

¹⁹F NMR (400 MHz, CDCl₃): δ ppm −63.0 (s, 3F).

LCMS Rt=3.44 minutes MS m/z No mass ion observed

### Preparation 71

### 4-(Benzyloxy)-3-bromo-3′-(trifluoromethyl)biphenyl

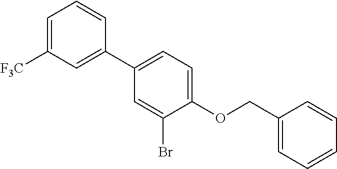 [SMILES: FC(F)(F)c1cccc(-c2ccc(OCc3ccccc3)c(Br)c2)c1]

To a solution of 3-Bromo-3′-(trifluoromethyl)biphenyl-4-ol (Preparation 39, 20.9 g, 65.9 mmol) in N,N-dimethylformamide (300 mL) at room temperature was added benzyl bromide (8.6 mL, 72.5 mmol) followed by potassium carbonate (18.2 g, 132 mmol). The reaction was stirred at room temperature for 4 hours, and partitioned between water and ethyl acetate (1/1, 1 L). The aqueous layer was washed with ethyl acetate (2×100 mL) and the combined organic phases were washed with brine (200 mL), dried over anhydrous magnesium sulfate and concentrated in vacuo. The crude compound was purified by silica gel column chromatography eluting with 5-10% EtOAc in heptanes to afford the title compound as a yellow oil (25.2 g, 94%).

¹H NMR (400 MHz, CDCl₃): δ ppm 5.20 (s, 2H), 7.00 (d, 1H), 7.30-7.60 (m, 8H), 7.65 (d, 1H), 7.75 (s, 1H), 7.80 (s, 1H).

¹⁹F NMR (400 MHz, CDCl₃): δ ppm −63.0 (s, 3F).

LCMS Rt=3.23 minutes MS m/z No mass ion observed

### Preparation 72

### tert-Butyl [(4-bromopyridin-2-yl)methyl]carbamate

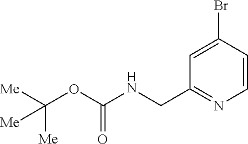 [SMILES: CC(C)(C)OC(=O)NCc1cc(Br)ccn1]

Di-tert-butyl dicarbonate (4 g, 18.28 mmol), triethylamine (3 mL, 21.93 mmol) and 4-dimethylaminopyridine (40 mg, 0.36 mmol) were added to a solution of 1-(4-bromopyridin-2-yl)methanamine (Preparation 73, 1.9 g, 10.1 mmol) in dichloromethane (40 mL) and the reaction mixture was stirred for 18 hours. The mixture was quenched with water (50 mL), extracted with dichloromethane (100 mL) and the organic layer was dried over magnesium sulfate and solvent was evaporated in vacuo. The residue was purified by silica gel column chromatography eluting with 1:1 ethyl acetate:heptane to give the title compound as a colourless oil (1.99 g, 95%).

¹H NMR (400 MHz, CDCl₃): δ ppm 1.46 (s, 9H), 4.42 (d, 2H), 5.45 (br s, 1H), 7.34-7.36 (m, 1H), 7.45 (s, 1H), 8.34 (d, 1H).

### Preparation 73

### 1-(4-bromopyridin-2-yl)methanamine

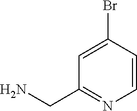

Hydrazine hydrate (4 mL, 82.19 mmol) was added to a suspension of 2-[(4-bromopyridin-2-yl)methyl]-1H-isoindole-1,3(2H)-dione (Preparation 74, 3.81 g, 12.02 mmol) in ethanol (100 mL) and the reaction mixture was heated at 70° C. for 3 hours. The reaction mixture was cooled and solvent was evaporated in vacuo, the solid residue was triturated with ethyl acetate (15 mL) and mother liquor was evaporated to give the title compound as brown oil (1.29 g, 57%).

¹H NMR (400 MHz, CDCl₃): δ ppm 3.96 (s, 2H), 7.29 (dd, 1H), 7.51 (d, 1H), 8.36 (d, 1H).

LCMS Rt=1.54 minutes MS m/z 188 [M+H]⁺

### Preparation 74

### 2-[(4-Bromopyridin-2-yl)methyl]-1H-isoindole-1,3(2H)-dione

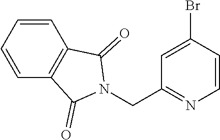 [SMILES: O=C1c2ccccc2C(=O)N1Cc1cc(Br)ccn1]

4-Bromo-2-(bromomethyl)pyridine hydrobromide (Preparation 75, 4.92 g, 14.86 mmol), phthalimide (2.19 g, 14.86 mmol) and potassium carbonate (4.11 g, 29.73 mmol) in N,N-dimethylformamide (50 mL) were stirred for 18 hours at room temperature. The reaction mixture was diluted with water (50 mL) and stirred for 5 minutes. The suspension was filtered and the solid was washed through with water followed by heptane then dried to give the title compound as brown solid (3.83 g, 81%).

¹H NMR (400 MHz, acetone-d₆): δ ppm 4.99 (s, 2H), 7.51 (dd, 1H), 7.71 (dd, 1H), 7.87-7.93 (m, 4H), 8.33 (d, 1H).

LCMS Rt=2.86 minutes MS m/z 318 [M+H]⁺

### Preparation 75

### 4-bromo-2-(bromomethyl)pyridine hydrobromide salt

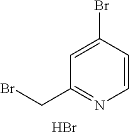

Phosphorous tribromide (15.6 mL, 164 mmol) was added to a solution of (4-bromopyridin-2-yl)methanol (Preparation 76, 5.12 g, 27.38 mmol) in dichloromethane (100 mL) at 0° C. and then heated to reflux for 18 hours. The reaction mixture was cooled, the suspension was poured into crushed ice (150 g) and basified to pH=10 with a saturated aqueous solution of potassium carbonate. The mixture was extracted with dichloromethane (3×100 mL), combined organic layers were dried over magnesium sulfate and the solvent was evaporated in vacuo. The dark oil residue was diluted with diethyl ether (60 mL), a solution of acetic acid/hydrogen bromide (48% solution) [1:1 (8 mL)] was added and the resulting solid was collected via filtration then dried to give the title compound as a brown solid (4.92 g, 54%).

¹H NMR (400 MHz, MeOD-d₄): δ ppm 4.80 (s, 2H), 8.20 (dd, 1H), 8.41 (d, 1H), 8.68 (d, 1H).

LCMS Rt=2.68 minutes MS m/z 252 [M+H]⁺

### Preparation 76

### (4-Bromopyridin-2-yl)methanol

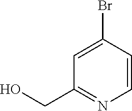

Trifluoroacetic anhydride (58.6 mL, 415 mmol) was added to a solution of 4-bromo-2-methylpyridine 1-oxide (Preparation 77, 15.6 g, 82.98 mmol) in dichloromethane (250 mL) at 0° C. (ice-bath). The ice-bath was removed and the reaction mixture was stirred at reflux for 12 hours. The reaction mixture was cooled and solvent was evaporated in vacuo. The dark yellow oil residue was diluted with dichloromethane (150 mL), 2M sodium hydroxide (100 mL) was added and the mixture was stirred vigorously for 18 hours. The layers were separated and the aqueous layer was further extracted with dichloromethane (50 mL), the combined organic layers were dried over magnesium sulfate and concentrated in vacuo to give the title compound as dark oil (10.24 g, 66%).

¹H NMR (400 MHz, CDCl₃): δ ppm 3.85 (br s, 1H), 4.75 (s, 2H), 7.37 (dd, 1H), 7.46 (s, 1H), 8.35 (d, 1H).

LCMS Rt=1.89 minutes MS m/z 189 [M+H]⁺

### Preparation 77

### 4-Bromo-2-methylpyridine 1-oxide

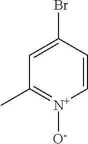

To a solution of 4-bromo-2-methylpyridine (20 g, 116.3 mmol) in dichloromethane (250 mL) was added meta-chloroperoxybenzoic acid (26 g, 151.2 mmol) at 0° C. (ice-bath). The ice-bath was removed and the reaction mixture was stirred at room temperature for 3 hours. The reaction mixture was washed with saturated sodium bicarbonate (100 mL), the aqueous layer was further extracted with dichloromethane (50 mL) and the combined organic layers were dried over magnesium sulfate and the solvent was evaporated in vacuo. The residue was dissolved in ethyl acetate (60 mL), filtered through a pad of silica gel eluting with ethyl acetate (60 mL) and the solvent was evaporated in vacuo to give the title compound as dark oil (15.92 g, 73%).

¹H NMR (400 MHz, CDCl₃): δ ppm 2.48 (s, 3H), 7.25 (dd, 1H), 7.40 (d, 1H), 8.08 (d, 1H).

LCMS Rt=1.49 minutes MS m/z 189 [M+H]⁺

### Preparation 78

### 3-Iodobiphenyl-4-ol

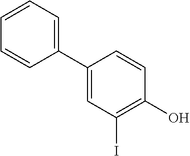 [SMILES: Oc1ccc(-c2ccccc2)cc1I]

To a solution of [1,1′-biphenyl]-4-ol (12.7 g, 74.6 mmol) in glacial acetic acid (250 mL), cooled to 0° C. was added N-iodosuccinimide (16.8 g, 74.6 mmol). The reaction mixture was stirred for 2 hours at 0° C. then allowed to slowly warm to room temperature, and was stirred for 18 hours at room temperature. The solution was concentrated in vacuo and the residue was partitioned between water (200 mL) and dichloromethane (200 mL). The aqueous layer was separated and extracted with dichloromethane (2×200 mL). The organic layers were combined, washed with a 0.5M aqueous solution of sodium thiosulfate (100 mL), dried over sodium sulfate, filtered and concentrated in vacuo. The crude material was purified by silica gel column chromatography eluting with 30% ethyl acetate in cyclohexane to afford the title compound as a pale yellow solid (12.8 g, 58%).

LCMS Rt=3.31 minutes MS m/z 295 [M−H]⁻

### Preparation 79

### 2-(1-Acetylpiperidin-4-yl)ethanamine

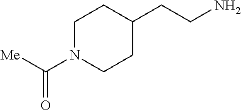 [SMILES: CC(=O)N1CCC(CCN)CC1]

To a solution of tert-butyl [2-(1-acetylpiperidin-4-yl)ethyl]carbamate (Preparation 80, 4.45 g, 16.45 mmol) in dioxane (50 mL) was added a 4M solution of hydrogen chloride in dioxane (30 mL, 120 mmol). The solution was stirred for 18 hours at room temperature and concentrated in vacuo. The crude material was dissolved in water (20 mL) and the solution was basified with a 30% aqueous solution of sodium hydroxide (20 mL). The aqueous solution was extracted with dichloromethane (3×50 mL). The organic layers were combined, dried over magnesium sulfate, filtered and concentrated in vacuo to afford the title compound as a clear oil (2.55 g, 91%).

¹H NMR (400 MHz, CDCl₃): δ ppm 1.10 (m, 2H), 1.40-1.30 (m, 4H), 1.57 (m, 1H), 1.71 (m, 2H), 2.06 (m, 3H), 2.51 (m, 1H), 2.73 (m, 2H), 3.01 (m, 1H), 3.77 (m, 1H), 4.57 (m, 1H).

LCMS Rt=2.99 minutes MS m/z 171 [M+H]⁺

### Preparation 80

### tert-Butyl [2-(1-acetylpiperidin-4-yl)ethyl]carbamate

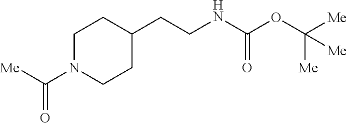 [SMILES: CC(=O)N1CCC(CCNC(=O)OC(C)(C)C)CC1]

To a solution of tert-butyl (2-piperidin-4-ylethyl)carbamate (Preparation 81, 4.0 g, 17.5 mmol) in dichloromethane (80 mL) was added triethylamine (4.90 mL, 35.0 mmol) and acetic anhydride (1.75 mL, 18.4 mmol). The reaction mixture was stirred for 1 hour at room temperature then water (20 mL) and 30% aqueous solution of sodium hydroxide (10 mL) were added and the mixture was stirred for 15 minutes. The organic layer was separated and the aqueous layer was extracted with dichloromethane (2×20 mL). The organic layers were combined, dried over magnesium sulfate, filtered and concentrated in vacuo. The crude material was azeotroped with ethyl acetate (50 mL) to afford the title compound as a clear viscous oil (4.45 g, 94%).

¹H NMR (400 MHz, CDCl₃): δ ppm 1.10 (m, 2H), 1.42-1.54 (m, 12H), 1.71 (m, 2H), 2.03 (s, 3H), 2.50 (m, 1H), 2.99 (m, 1H), 3.14 (m, 2H), 3.77 (m, 1H), 4.45 (m, 2H).

LCMS Rt=2.43 minutes MS m/z 271 [M+H]⁺

### Preparation 81

### tert-Butyl (2-piperidin-4-ylethyl)carbamate

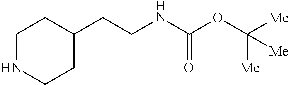 [SMILES: CC(C)(C)OC(=O)NCCC1CCNCC1]

To a solution of tert-butyl (2-pyridin-4-ylethyl)carbamate (Preparation 82, 26.9 g, 120.9 mmol) in methanol (370 mL), cooled to 0° C., was added a 6N aqueous solution of hydrochloric acid (20.2 mL, 120.9 mmol). The solution was subject to hydrogenation using platinum (IV) oxide (1.37 g, 6.05 mmol) and hydrogen (1000 psi) over 24 hours at room temperature. The reaction mixture was filtered on a pad of Arbocel which was washed successively with methanol (300 mL) and water (100 mL). The filtrate was concentrated in vacuo. The crude material was diluted with a saturated aqueous solution of sodium bicarbonate (150 mL). The solution was extracted with dichloromethane (50 mL). The organic layer was extracted with water (2×50 mL). The aqueous layers were combined, basified with a 30% aqueous solution of sodium hydroxide (30 mL) and extracted with dichloromethane (8×200 mL). The organic layers were combined, dried over sodium sulfate, filtered and concentrated in vacuo to afford the title compound as a clear viscous oil (26.8 g, 97%).

¹H NMR (400 MHz, CDCl₃): δ ppm 1.17 (m, 2H), 1.41 (m, 10H), 1.68 (m, 2H), 2.58 (m, 2H), 3.09 (m, 6H), 4.53 (s, 1H).

LCMS Rt=1.73 minutes MS m/z 229 [M+H]⁺

### Preparation 82

### tert-Butyl (2-pyridin-4-ylethyl)carbamate

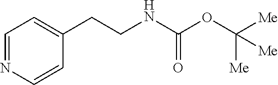 [SMILES: CC(C)(C)OC(=O)NCCc1ccncc1]

To a solution of 2-(pyridin-4-yl)ethanamine (14.77 g, 120.9 mmol) in dichloromethane (150 mL) was slowly added di-tert-butyl dicarbonate (27.7 g, 126.9 mmol). The reaction mixture was stirred for 2 hours at room temperature and the solution was concentrated in vacuo to afford the title compound as an orange oil (28.48 g, >100%).

¹H NMR (400 MHz, CDCl₃): δ ppm 1.40 (s, 9H), 2.77 (m, 2H), 3.36 (m, 2H), 4.74 (s, 1H), 7.09 (m, 2H), 8.46 (m, 2H).

LCMS Rt=2.38 minutes MS m/z 223 [M+H]⁺

### Preparation 83

### 3-((2,5,8,11-tetraoxatridecan-13-yl)oxy)azetidine trifluoroacetate salt

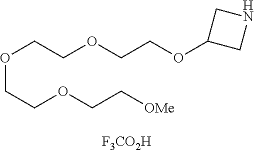

A solution of tert-butyl 3-((2,5,8,11-tetraoxatridecan-13-yl)oxy)azetidine-1-carboxylate (Preparation 63, 94 mg, 0.26 mmol) in TFA (0.5 mL, 7 mmol) was warmed to 50° C. for 2 hours. The reaction mixture was cooled to room temperature and concentrated in vacuo to afford the title compound (98 mg, crude quant.)

LCMS Rt=0.28 minutes MS m/z 264 [M+H]⁺

### Preparation 84

### tert-butyl [(4-{4-[2-chloro-5-fluoro-4-(1,3,4-thiadiazol-2-ylsulfamoyl)phenoxy]-3′-(trifluoromethyl) biphenyl-3-yl}pyridin-2-yl)methyl][2-(piperidin-4-yl)ethyl]carbamate

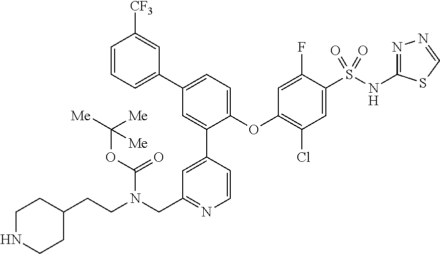 [SMILES: CC(C)(C)OC(=O)N(CCC1CCNCC1)Cc1cc(-c2cc(-c3cccc(C(F)(F)F)c3)ccc2Oc2cc(F)c(S(=O)(=O)Nc3nncs3)cc2Cl)ccn1]

The title compound was prepared according to the methods described by Example 2 followed by Preparation 6 followed by Preparation 5 using 2,2,2-trichloroethyl [(4-{4-[2-chloro-5-fluoro-4-(1,3,4-thiadiazol-2-ylsulfamoyl)phenoxy]-3′-(trifluoromethyl)biphenyl-3-yl}pyridin-2-yl)methyl]{2-[1-(trifluoroacetyl) piperidin-4-yl]ethyl}carbamate (Preparation 85).

¹H NMR (400 MHz, CDCl₃): δ ppm 0.90-1.03 (m, 3H), 1.27-1.43 (m, 13H), 2.80 (t, 2H), 3.10 (d, 2H), 3.44 (d, 2H), 4.60 (d, 2H), 6.32 (d, 1H), 7.16 (br s, 1H), 7.35 (d, 1H), 7.51-7.69 (m, 5H), 7.78 (d, 1H), 7.84 (s, 1H), 7.86 (d, 1H), 8.40 (s, 1H), 8.58 (d, 1H).

MS m/z 847 [M+H]⁺

### Preparation 85

### 2,2,2-trichloroethyl [(4-{4-[2-chloro-5-fluoro-4-(1,3,4-thiadiazol-2-ylsulfamoyl)phenoxy]-3′-(trifluoromethyl)biphenyl-3-yl}pyridin-2-yl)methyl]{2-[1-(trifluoroacetyl)piperidin-4-yl]ethyl}carbamate

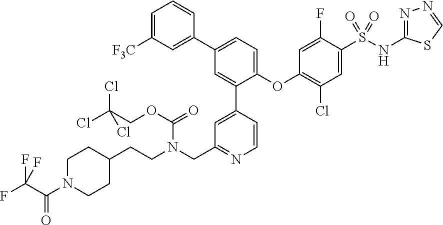 [SMILES: O=C(OCC(Cl)(Cl)Cl)N(CCC1CCN(C(=O)C(F)(F)F)CC1)Cc1cc(-c2cc(-c3cccc(C(F)(F)F)c3)ccc2Oc2cc(F)c(S(=O)(=O)Nc3nncs3)cc2Cl)ccn1]

The title compound was prepared according to the method described for Preparation using 2,2,2-trichloroethyl [(4-{4-[2-chloro-5-fluoro-4-(1,3,4-thiadiazol-2-ylsulfamoyl) phenoxy]-3′-(trifluoromethyl)biphenyl-3-yl}pyridin-2-yl)methyl][2-(piperidin-4-yl)ethyl]carbamate (Preparation 86) and taken on crude directly to the next step.

### Preparation 86

### 2,2,2-trichloroethyl [(4-{4-[2-chloro-5-fluoro-4-(1,3,4-thiadiazol-2-ylsulfamoyl)phenoxy]-3′-(trifluoromethyl)biphenyl-3-yl}pyridin-2-yl)methyl][2-(piperidin-4-yl)ethyl]carbamate

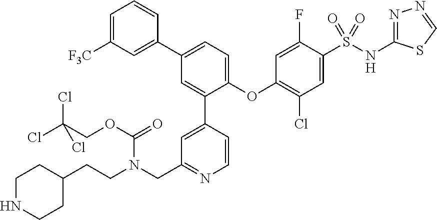 [SMILES: O=C(OCC(Cl)(Cl)Cl)N(CCC1CCNCC1)Cc1cc(-c2cc(-c3cccc(C(F)(F)F)c3)ccc2Oc2cc(F)c(S(=O)(=O)Nc3nncs3)cc2Cl)ccn1]

The title compound was prepared according to the methods described for Preparation 11 followed by Preparation 10 using tert-butyl 4-(2-{({4-[4-hydroxy-3′-(trifluoromethyl) biphenyl-3-yl]pyridin-2-yl}methyl)[(2,2,2-trichloroethoxy)carbonyl]amino}ethyl)piperidine-1-carboxylate (Preparation 26) and 5-chloro-N-(2,4-dimethoxybenzyl)-2,4-difluoro-N-(1,3,4-thiadiazol-2-yl)benzenesulfonamide (WO2010079443).

¹H NMR (400 MHz, CDCl₃): δ ppm 0.78-0.84 (m, 2H), 0.94-0.97 (m, 2H), 1.21-1.36 (m, 3H), 2.78 (t, 2H), 3.14-3.27 (m, 2H), 3.37-3.40 (m, 2H), 4.46-4.77 (m, 4H), 6.27-6.34 (m, 1H), 7.01-7.15 (m, 1H), 7.42-7.73 (m, 6H), 7.79 (d, 1H), 7.84 (s, 1H), 7.94 (d, 1H), 8.45 (d, 1H), 8.61 (d, 1H).

MS m/z 921 [M+H]⁺

### Preparation 87

### 5-chloro-2-fluoro-4-({3-[2-(piperazin-1-yl)pyridin-4-yl]-4′-(trifluoromethyl) biphenyl-4-yl}oxy)-N-(1,3,4-thiadiazol-2-yl)benzenesulfonamide

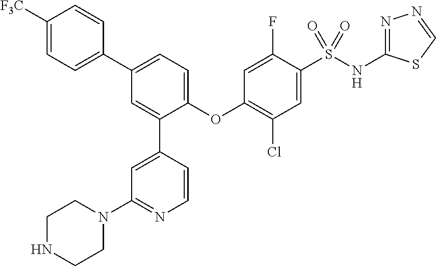 [SMILES: O=S(=O)(Nc1nncs1)c1cc(Cl)c(Oc2ccc(-c3ccc(C(F)(F)F)cc3)cc2-c2ccnc(N3CCNCC3)c2)cc1F]

The title compound was prepared according to the methods described by Preparation 8 followed by Preparation 2 using tert-butyl 4-(4-(4-hydroxy-4′-(trifluoromethyl)-[1,1′-biphenyl]-3-yl)pyridin-2-yl)piperazine-1-carboxylate (WO2012004743) and 5-chloro-N-(2,4-dimethoxybenzyl)-2,4-difluoro-N-(1,3,4-thiadiazol-2-yl)benzenesulfonamide (WO2010079443). The title compound was isolated as the free parent following elution through an SCX column using 7N ammonia in methanol.

¹H NMR (400 MHz, DMSO-d₆): δ ppm 3.05-3.07 (m, 4H), 3.65-3.67 (m, 4H), 6.75 (d, 1H), 6.90 (dd, 1H), 6.99 (s, 1H), 7.31 (d, 1H), 7.71 (d, 1H), 7.81-7.87 (m, 4H), 7.98 (d, 2H), 8.11 (d, 1H), 8.55 (s, 1H).

MS m/z 689 [M−H]⁻

### Preparation 88

### tert-butyl [(4-{4-[2-chloro-5-fluoro-4-(1,3-thiazol-4-ylsulfamoyl)phenoxy]-3′-(trifluoromethyl) biphenyl-3-yl}pyridin-2-yl)methyl][2-(piperidin-4-yl)ethyl]carbamate

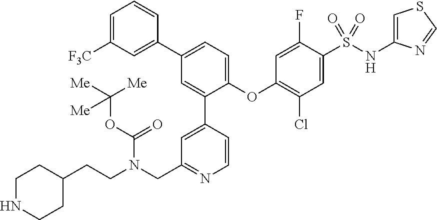 [SMILES: CC(C)(C)OC(=O)N(CCC1CCNCC1)Cc1cc(-c2cc(-c3cccc(C(F)(F)F)c3)ccc2Oc2cc(F)c(S(=O)(=O)Nc3cscn3)cc2Cl)ccn1]

The title compound was prepared according to the method described by Preparation 5 using tert-butyl [(4-{4-[2-chloro-5-fluoro-4-(1,3-thiazol-4-ylsulfamoyl)phenoxy]-3′-(trifluoromethyl) biphenyl-3-yl}pyridin-2-yl)methyl]{2-[1-(trifluoroacetyl)piperidin-4-yl]ethyl}carbamate (Preparation 89) and purified using reverse phase column chromatography eluting with 0-100% water:MeCN with 0.1% formic acid.

¹H NMR (400 MHz, CDCl₃): δ ppm 1.20-1.40 (m, 14H), 1.60 (m, 2H), 2.70 (t, 2H), 3.10 (m, 2H), 3.40 (m, 2H), 4.50 (d, 2H), 6.30 (d, 1H), 6.80 (s, 1H), 7.20 (m, 1H), 7.30 (m, 1H), 7.40 (s, 1H), 7.60 (m, 3H), 7.75 (m, 1H), 7.80 (s, 1H), 7.85 (d, 1H), 8.45 (s, 1H), 8.59 (s, 1H), 8.60 (s, 1H).

MS m/z 846 [M+H]⁺

### Preparation 89

### tert-butyl [(4-{4-[2-chloro-5-fluoro-4-(1,3-thiazol-4-ylsulfamoyl)phenoxy]-3′-(trifluoromethyl)biphenyl-3-yl}pyridin-2-yl)methyl]{2-[1-(trifluoroacetyl)piperidin-4-yl]ethyl}carbamate

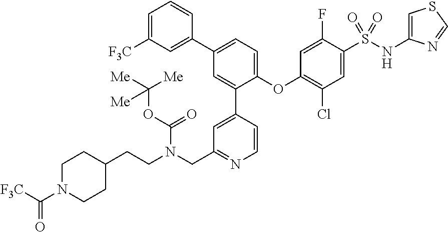 [SMILES: CC(C)(C)OC(=O)N(CCC1CCN(C(=O)C(F)(F)F)CC1)Cc1cc(-c2cc(-c3cccc(C(F)(F)F)c3)ccc2Oc2cc(F)c(S(=O)(=O)Nc3cscn3)cc2Cl)ccn1]

The title compound was prepared according to the method described for Preparation 6 using 5-chloro-2-fluoro-N-(1,3-thiazol-4-yl)-4-{[3-{2-[({2-[1-(trifluoroacetyl)piperidin-4-yl]ethyl}amino)methyl]pyridin-4-yl}-3′-(trifluoromethyl) biphenyl-4-yl]oxy}benzenesulfonamide (Example 35).

¹H NMR (400 MHz, CDCl₃): δ ppm 1.30-1.50 (m, 12H), 1.80 (m, 2H), 2.60 (t, 2H), 3.00 (m, 2H), 3.20 (m, 2H), 3.95 (d, 1H), 4.40 (m, 3H), 6.40 (d, 1H), 6.60 (s, 1H), 7.05 (d, 1H), 7.40 (m, 2H), 7.55-7.65 (m, 4H), 7.75 (d, 1H), 7.80 (s, 1H), 7.95 (d, 1H), 8.45 (s, 1H), 8.50 (m, 1H).

MS m/z 942 [M+H]⁺

The ability of the compounds of formula (I) to block the Nav1.7 (or SCN9A) channel was measured using the assay described below.

Cell Line Construction and Maintenance

Human Embryonic Kidney (HEK) cells were transfected with an hSCN9A construct using lipofectamine reagent (Invitrogen), using standard techniques. Cells stably expressing the hSCN9A constructs were identified by their resistance to G-418 (400 μg/ml). Clones were screened for expression using the whole-cell voltage-clamp technique.

Cell Culture

HEK cells stably transfected with hSCN9A were maintained in DMEM medium supplemented with 10% heat-inactivated fetal bovine serum and 400 μg/ml G-418 in an incubator at 37° C. with a humidified atmosphere of 10% CO₂. For HTS, cells were harvested from flasks by trypsinization and replated in an appropriate multi-well plate (typically 96 or 384 wells/plate) such that confluence would be achieved within 24 hours of plating. Cells were typically used for electrophysiological experiments within 24 to 72 hours after plating.

Electrophysiological Recording

For conventional whole-cell voltage clamp experiments cells were removed from the culture flask by brief trypsinization and re-plated at low density onto glass cover slips. Cover slips containing HEK cells expressing hSCN9A were placed in a bath on the stage of an inverted microscope and perfused (approximately 1 ml/minutes) with extracellular solution of the following composition: 138 mM NaCl, 2 mM CaCl₂, 5.4 mM KCl, 1 mM MgCl₂, 10 mM glucose, and 10 mM HEPES, pH 7.4, with NaOH. Pipettes were filled with an intracellular solution of the following composition: 135 mM CsF, 5 mM CsCl, 2 mM MgCl₂, 10 mM EGTA, 10 mM HEPES, pH 7.3 with NaOH, and had a resistance of 1 to 2 megaohms. The osmolarity of the extracellular and intracellular solutions was 300 mOsm/kg and 295 mOsm/kg, respectively. All recordings were made at room temperature (22-24° C.) using AXOPATCH 200B amplifiers and PCLAMP software (Axon Instruments, Burlingame, Calif.). hSCN9A currents in HEK cells were measured using the whole-cell configuration of the patch-clamp technique (Hamill et al., 1981). Uncompensated series resistance was typically 2 to 5 mega ohms and >85% series resistance compensation was routinely achieved. As a result, voltage errors were negligible and no correction was applied. Current records were acquired at 20 to 50 KHz and filtered at 5 to 10 KHz.

HEK cells stably transfected with hSCN9A were viewed under Hoffman contrast optics and placed in front of an array of flow pipes emitting either control or compound-containing extracellular solutions.

The voltage-dependence of inactivation was determined by applying a series of depolarizing prepulses (8 sec long in 10 mV increments) from a negative holding potential. The voltage was then immediately stepped to 0 mV to assess the magnitude of the sodium current. Currents elicited at 0 mV were plotted as a function of prepulse potential to allow estimation of the voltage at which 50% of the channels were inactivated (midpoint of inactivation or V1/2). Compounds were tested for their ability to inhibit hSCN9A sodium channels by activating the channel with a 20 msec voltage step 15 to 0 mV following an 8 second conditioning prepulse to the empirically determined V1/2. Compound effect (% inhibition) was determined by difference in current amplitude before and after application of test compounds. For ease of comparison, “estimated IC-50” (EIC₅₀) values were calculated from single point electrophysiology data by the following equation, (tested concentration, nM)×(100-% inhibition/% inhibition). Inhibition values <20% and >80% were excluded from the calculation.

Electrophysiological assays were conducted with PatchXpress 7000 hardware and associated software (Molecular Devices Corp). All assay buffers and solutions were identical to those used in conventional whole-cell voltage clamp experiments described above. hSCN9A cells were grown as above to 50%-80% confluency and harvested by trypsinization. Trypsinized cells were washed and resuspended in extracellular buffer at a concentration of 1×10⁶ cells/ml. The onboard liquid handling facility of the PatchXpress was used for dispensing cells and application of test compounds. Determination of the voltage midpoint of inactivation was as described for conventional whole-cell recordings. Cells were then voltage-clamped to the empirically determined V1/2 and current was activated by a 20 msec voltage step to 0 mV. For ease of comparison, “estimated IC-50” (EIC₅₀) values were calculated from single point electrophysiology data by the following equation, (tested concentration, nM)×(100-% inhibition/% inhibition). Inhibition values <20% and >80% were excluded from the calculation.

Electrophysiological assays may also be conducted using the Ionworks Quattro automated electrophysiological platform (Molecular Devices Corp). Intracellular and extracellular solutions were as described above with the following changes, 100 μg/ml amphotericin was added to the intracellular solution to perforate the membrane and allow electrical access to the cells. hSCN9A cells were grown and harvested as for PatchXpress and cells were resuspended in extracellular solution at a concentration of 3-4×10⁶ cells/ml. The onboard liquid handling facility of the Ionworks Quattro was used for dispensing cells and application of test compounds. A voltage protocol was then applied that comprised of a voltage step to fully inactivate the sodium channels, followed by a brief hyperpolarized recovery period to allow partial recovery from inactivation for unblocked sodium channels, followed by a test depolarized voltage step to assess magnitude of inhibition by test compound. Compound effect was determined based on current amplitude difference between the pre-compound addition and post-compound addition scans.

All compounds were dissolved in dimethyl sulfoxide to make 10 mM stock solutions, which were then diluted into extracellular solution to attain the final concentrations desired. The final concentration of dimethyl sulfoxide (<0.3% dimethyl sulfoxide) was found to have no significant effect on hSCN9A sodium currents.

Unless otherwise stated, the PatchXpress (PX) platform was used to test compounds of the Examples, which were found to have the Nav1.7 EIC₅₀ (nM) values specified in the table below.

| Ex. | EIC₅₀ |
|---|---|
| 1 | 5.8 |
| 2 | >30 ¹ |
| 3 | 1.0 |
| 4 | ND |
| 5 | 1.5 |
| 6 | 2.1 |
| 7 | 1.5 |
| 8 | 3.4 |
| 9 | 10.9 |
| 10 | 4.7 |
| 11 | 10.4 |
| 12 | 2.3 |
| 13 | 13.0 |
| 14 | 5.0 |
| 15 | 1.0 |
| 16 | 0.9 |
| 17 | <10 ² |
| 18 | 0.58 |
| 19 | 17.5 |
| 20 | 1.0 |
| 21 | 7.9 |
| 22 | 2.6 |
| 23 | 5.0 |
| 24 | 13.4 |
| 25 | ND |
| 26 | 0.50 |
| 27 | 47.2 |
| 28 | 241.0 |
| 29 | 2.9 |
| 30 | 23.9 |
| 31 | 9.8 |
| 32 | 16.0 |
| 33 | 1.7 |
| 34 | ND |
| 35 | ND |

¹ 10.71% inhibition at 30 nM dose
² 87.38% inhibition at 10 nM dose
ND = no data

Using the above described assay and the PX platform, preferred compounds of the invention have a Nav1.7 EIC₅₀ (nM) value of <10, such as <5, e.g. <1.

The ability of compounds of formula (I) to block the Nav1.5 (or SCN5A) channel can also be measured using an assay analogous to that described above but replacing the SCN9A gene with the SCN5A gene. All other conditions remain the same including the same cell line and conditions for cell growth. The estimated IC50s are determined at the half inactivation for Nav1.5. These results can be compared to the EIC₅₀ value at the Nav1.7 channel to determine the selectivity of a given compound for Nav1.7 vs Nav1.5.

Solubility Data

Aqueous solubility data were generated via a “shake-flask” method where an excess of compound was added to a buffer (typically phosphate buffered saline at pH 7.4) and shaken for a period of 18 hours at room temperature. After this time any excess solid was removed by double centrifugation to obtain a saturated aqueous solution. The amount of compound solubilised was quantified by HPLC-UV or LC-MS against a standard calibration curve.

The solubility of the compounds of Examples 12, 15, 17 and 18 was assessed using the above method.

The solubility data generated are set out below. Where a greater than figure is quoted, all available compound was solubilised and saturation was not achieved in the conditions employed.

| Ex No. | pH | Solubility/μg/mL |
|---|---|---|
| 6 | 5.0 | >2300 |
| | 7.5 | <500 |
| 7 | 7.2 | 0.5 |
| 12 | 7.5 | 612 |
| 15 | 7.5 | 110 |
| 17 | 7.4 | >2300 |
| 18b | 7.4 | >10000 |
| 19 | 7.5 | >3 |
| 21 | 7.5 | 56 |
| 26 | 7.5 | 3 |
| 29 | 7.0 | <0.5 |

1. A compound of formula (I): 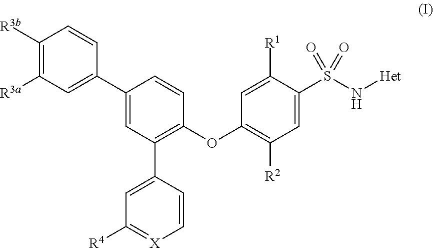
or a pharmaceutically acceptable salt thereof, wherein:
Het is ‘C-linked’ thiazolyl or thiadiazolyl;
X is CH or N;
R¹ is H or F;
R² is Cl or CN;
R^{3a} is H or CF₃;
R^{3b} is H or, when R^{3a} is H, may also be CF₃;
R⁴ is 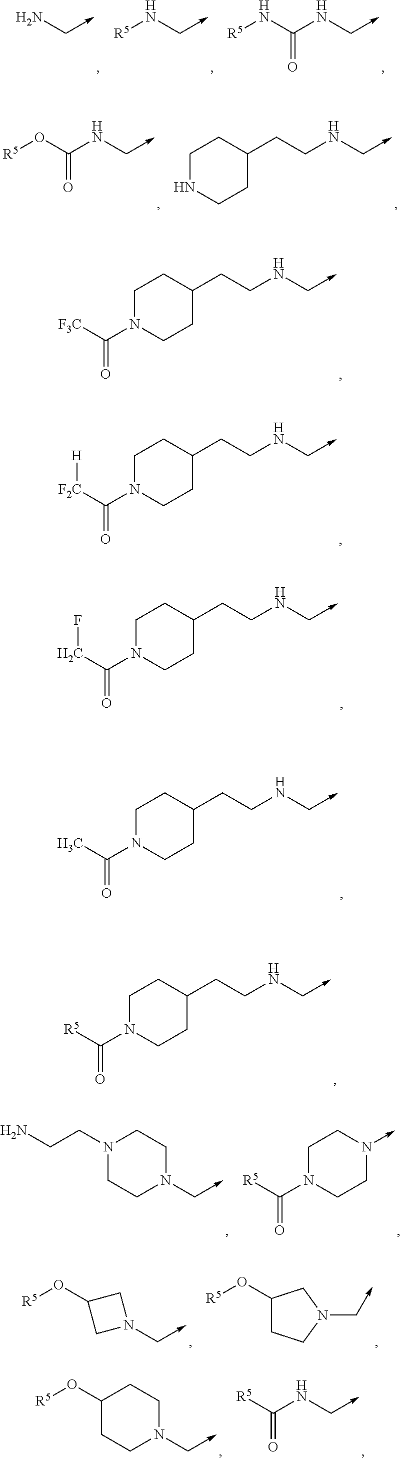 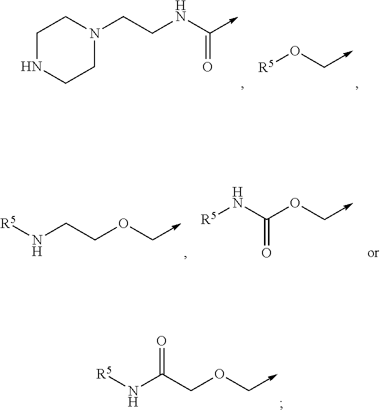
R⁵ is CH₃—(OC₂H₄)n-; and
n is 1 to 15.

2. A compound according to claim 1 or a pharmaceutically acceptable salt thereof wherein Het is ‘C-linked’ thiadiazolyl.

3. A compound according to claim 1 or a pharmaceutically acceptable salt thereof, wherein X is N.

4. A compound according to claim 1 or a pharmaceutically acceptable salt thereof, wherein R¹ is H.

5. A compound according to claim 1 or a pharmaceutically acceptable salt thereof, wherein R² is CN.

6. A compound according to claim 1 or a pharmaceutically acceptable salt thereof, wherein R^{3a} is CF₃ and R^{3b} is H.

7. A compound according to claim 1 or a pharmaceutically acceptable salt thereof, wherein R⁴ is

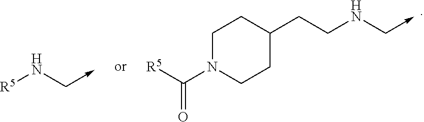

8. A compound according to claim 1 or a pharmaceutically acceptable salt thereof, wherein n is 4.

9. A compound according to claim 1 or a pharmaceutically acceptable salt thereof, wherein n is 12.

10. A compound according to claim 1 or a pharmaceutically acceptable salt thereof wherein R⁴ is

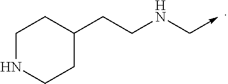

11. The compound according to claim 1, or a pharmaceutically acceptable salt thereof, that is:
4-({3-[2-({[2-(1-Acetylpiperidin-4-yl)ethyl]amino}methyl)pyridin-4-yl]-3′-(trifluoromethyl)biphenyl-4-yl}oxy)-3-cyano-N-1,2,4-thiadiazol-5-ylbenzenesulfonamide;
5-Chloro-2-fluoro-4-{[3-(2-{[(2-piperidin-4-ylethyl)amino]methyl}pyridin-4-yl)-3′-(trifluoromethyl)biphenyl-4-yl]oxy}-N-1,3-thiazol-4-ylbenzenesulfonamide;
6′-{2-cyano-4-[(1,2,4-thiadiazol-5-ylamino)sulfonyl]phenoxy}-N-(2-piperazin-1-ylethyl)-1,1′:3′,1″-terphenyl-3-carboxamide;
4-[(3″-{[4-(2-aminoethyl)piperazin-1-yl]methyl}-1,1′:3′,1″-terphenyl-4′-yl)oxy]-3-cyano-N-1,2,4-thiadiazol-5-ylbenzenesulfonamide;
3-Cyano-4-[(3″-{[(2-piperidin-4-ylethyl)amino]methyl}-1,1′:3′,1″-terphenyl-4′-yl)oxy]-N-1,2,4-thiadiazol-5-ylbenzenesulfonamide;
5-Chloro-2-fluoro-4-{[3-(2-{[(2-piperidin-4-ylethyl)amino]methyl}pyridin-4-yl)-3′-(trifluoromethyl)biphenyl-4-yl]oxy}-N-1,3,4-thiadiazol-2-ylbenzenesulfonamide;
4-({3-[2-(Aminomethyl)pyridin-4-yl]-3′-(trifluoromethyl)biphenyl-4-yl}oxy)-5-chloro-2-fluoro-N-1,3,4-thiadiazol-2-ylbenzenesulfonamide;
2-[2-(2-methoxyethoxy)ethoxy]ethyl [(4-{4-[2-cyano-4-(1,2,4-thiadiazol-5-ylsulfamoyl)phenoxy]-3′-(trifluoromethyl)biphenyl-3-yl}pyridin-2-yl)methyl]carbamate;
3-cyano-4-((3-(2-(3-oxo-7,10,13,16-tetraoxa-2,4-diazaheptadecyl)pyridin-4-yl)-3′-(trifluoromethyl)-[1,1′-biphenyl]-4-yl)oxy)-N-(1,2,4-thiadiazol-5-yl)benzenesulfonamide;
4-((3-(2-(2,8,11,14,17-pentaoxa-5-azaoctadecyl)pyridin-4-yl)-3′-(trifluoromethyl)-[1,1′-biphenyl]-4-yl)oxy)-3-cyano-N-(1,2,4-thiadiazol-5-yl)benzenesulfonamide;
(4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methyl(2,5,8,11-tetraoxatridecan-13-yl)carbamate;
3-cyano-4-({3-[2-(2,5,8,11,14,17,20,23,26,29,32,35-dodecaoxa-38-azanonatriacontan-39-yl)pyridin-4-yl]-3′-(trifluoromethyl)biphenyl-4-yl}oxy)-N-(1,2,4-thiadiazol-5-yl)benzenesulfonamide;
3-cyano-4-({3-[2-(2,5,8,11,14-pentaoxapentadec-1-yl)pyridin-4-yl]-3′-(trifluoromethyl)biphenyl-4-yl}oxy)-N-(1,2,4-thiadiazol-5-yl)benzenesulfonamide;
2-((4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methoxy)-N-(2,5,8,11-tetraoxatridecan-13-yl)acetamide;
4-((3-(2-(5,8,11,14-tetraoxa-2-azapentadecyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-{[3″-({[2-(1-Acetylpiperidin-4-yl)ethyl]amino}methyl)-1′:3′,1″-terphenyl-4′-yl]oxy}-3-cyano-N-1,2,4-thiadiazol-5-ylbenzenesulfonamide;
4-((3-(2-(((2-(1-(2,5,8,11,14,17,20,23,26,29,32,35-dodecaoxaoctatriacontan-38-oyl)piperidin-4-yl)ethyl)amino)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
3-Cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide;
N-((4-(4-(4-(N-(1,2,4-thiadiazol-5-yl)sulfamoyl)-2-cyanophenoxy)-3′-(trifluoromethyl)-[1,1′-biphenyl]-3-yl)pyridin-2-yl)methyl)-2,5,8,11,14,17,20,23,26,29,32,35-dodecaoxaoctatriacontan-38-amide;
4-((3-(2-((3-((2,5,8,11-tetraoxatridecan-13-yl)oxy)azetidin-1-yl)methyl)pyridin-4-yl)-3′-(trifluoromethyl)-[1,1′-biphenyl]-4-yl)oxy)-3-cyano-N-(2,4-dimethoxybenzyl)-N-(1,2,4-thiadiazol-5-yl)benzenesulfonamide;
(R)-4-((3-(2-((3-((2,5,8,11-tetraoxatridecan-13-yl)oxy)pyrrolidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
(S)-4-((3-(2-((3-((2,5,8,11-tetraoxatridecan-13-yl)oxy)pyrrolidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-((3-(2-((4-((2,5,8,11-tetraoxatridecan-13-yl)oxy)piperidin-1-yl)methyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-((3-(2-(4-(2,5,8,11,14,17,20,23,26,29,32,35-dodecaoxaoctatriacontan-38-oyl)piperazin-1-yl)pyridin-4-yl)-4′-(trifluoromethyl)-[1,1′-biphenyl]-4-yl)oxy)-3-cyano-N-(1,2,4-thiadiazol-5-yl)benzenesulfonamide
3-Cyano-N-1,2,4-thiadiazol-5-yl-4-{[3-{2-[({2-[1-(trifluoroacetyl)piperidin-4-yl]ethyl}amino)methyl]pyridin-4-yl}-3′-(trifluoromethyl)biphenyl-4-yl]oxy}benzenesulfonamide;
4-({3-[2-(Aminomethyl)pyridin-4-yl]-3′-(trifluoromethyl)biphenyl-4-yl}oxy)-3-cyano-N-1,2,4-thiadiazol-5-ylbenzenesulfonamide;
N-[(4-{4-[2-chloro-5-fluoro-4-(1,3,4-thiadiazol-2-ylsulfamoyl)phenoxy]-3′-(trifluoromethyl)biphenyl-3-yl}pyridin-2-yl)methyl]-2,5,8,11,14,17,20,23,26,29,32,35-dodecaoxaoctatriacontan-38-amide;
N-[(4-{4-[2-chloro-5-fluoro-4-(1,3-thiazol-4-ylsulfamoyl)phenoxy]-3′-(trifluoromethyl)biphenyl-3-yl}pyridin-2-yl)methyl]-2,5,8,11,14,17,20,23,26,29,32,35-dodecaoxaoctatriacontan-38-amide;
4-({3-[2-(aminomethyl)pyridin-4-yl]-3′-(trifluoromethyl)biphenyl-4-yl}oxy)-5-chloro-2-fluoro-N-(1,3-thiazol-4-yl)benzenesulfonamide;
5-chloro-2-fluoro-4-{[3-{2-[({2-[1-(38-oxo-2,5,8,11,14,17,20,23,26,29,32,35-dodecaoxaoctatriacontan-38-yl)piperidin-4-yl]ethyl}amino)methyl]pyridin-4-yl}-3′-(trifluoromethyl)biphenyl-4-yl]oxy}-N-(1,3-thiazol-4-yl)benzenesulfonamide;
3-cyano-4-({3″-[({2-[1-(38-oxo-2,5,8,11,14,17,20,23,26,29,32,35-dodecaoxaoctatriacontan-38-yl)piperidin-4-yl]ethyl}amino)methyl]-1,1′:3′,1″-terphenyl-4′-yl}oxy)-N-(1,2,4-thiadiazol-5-yl)benzenesulfonamide;
5-chloro-2-fluoro-4-{[3-{2-[4-(38-oxo-2, 5,8,11,14,17,20,23,26,29,32,35-dodecaoxaoctatriacontan-38-yl)piperazin-1-yl]pyridin-4-yl}-4′-(trifluoromethyl)biphenyl-4-yl]oxy}-N-(1,3,4-thiadiazol-2-yl)benzenesulfonamide;
5-chloro-2-fluoro-4-{[3-{2-[({2-[1-(38-oxo-2,5,8,11,14,17,20,23,26,29,32,35-dodecaoxaoctatriacontan-38-yl)piperidin-4-yl]ethyl}amino)methyl]pyridin-4-yl}-3′-(trifluoromethyl)biphenyl-4-yl]oxy}-N-(1,3,4-thiadiazol-2-yl)benzenesulfonamide;
3-cyano-4-({3-[2-(2,5,8,11,14,17,20,23,26,29,32,35-dodecaoxa-38-azanonatriacontan-39-yl)pyridin-4-yl]-3′-(trifluoromethyl)biphenyl-4-yl}oxy)-N-(1,3,4-thiadiazol-2-yl)benzenesulfonamide; or
5-chloro-2-fluoro-N-(1,3-thiazol-4-yl)-4-{[3-{2-[({2-[1-(trifluoroacetyl)piperidin-4-yl]ethyl}amino)methyl]pyridin-4-yl}-3′-(trifluoromethyl)biphenyl-4-yl]oxy}benzenesulfonamide.

12. The compound according to claim 1, or a pharmaceutically acceptable salt thereof, that is:
3-cyano-4-({3-[2-(2,5,8,11,14,17,20,23,26,29,32,35-dodecaoxa-38-azanonatriacontan-39-yl)pyridin-4-yl]-3′-(trifluoromethyl)biphenyl-4-yl}oxy)-N-(1,2,4-thiadiazol-5-yl)benzenesulfonamide;
4-((3-(2-(5,8,11,14-tetraoxa-2-azapentadecyl)pyridin-4-yl)-3′-(trifluoromethyl)-[1,1′-biphenyl]-4-yl)oxy)-3-cyano-N-(1,2,4-thiadiazol-5-yl)benzenesulfonamide;
4-((3-(2-(((2-(1-(2,5,8,11,14,17,20,23,26,29,32,35-dodecaoxaoctatriacontan-38-oyl)piperidin-4-yl)ethyl)amino)methyl)pyridin-4-yl)-3′-(trifluoromethyl)-[1,1′-biphenyl]-4-yl)oxy)-3-cyano-N-(1,2,4-thiadiazol-5-yl)benzenesulfonamide; or
3-cyano-4-{[3-(2-{[(2-piperidin-4-ylethyl)amino]methyl}pyridin-4-yl)-3′-(trifluoromethyl)biphenyl-4-yl]oxy}-N-1,2,4-thiadiazol-5-ylbenzenesulfonamide.

13. A pharmaceutical composition comprising a compound according to claim 1 and a pharmaceutically acceptable excipient.

14. A pharmaceutical composition according to claim 13 including one or more additional therapeutic agents.

15. (canceled)

16. (canceled)

17. (canceled)

18. (canceled)

19. A method of treating a disorder in a human or animal for which a Nav1.7 inhibitor is indicated, comprising administering to said human or animal a therapeutically effective amount of a compound according to claim 1.
